(19) United States
(12) Patent Application Publication
(10) Pub. No.: US 2018/0073075 A1
(43) Pub. Date: Mar. 15, 2018

(54) Risk Factors of Cigarette Smoke-Induced Spriometric Phenotypes

(71) Applicant: LineaGen, Inc., Salt Lake City, UT (US)

(72) Inventors: Bradley Todd Webb, Richmond, VA (US); Barbara K. Zedler, Richmond, VA (US); Edward Lenn Murrelle, Midlothian, VA (US); Mark Leppert, Salt Lake City, UT (US); Edwin J. C. G. van den Oord, Richmond, VA (US); Daniel E. Adkins, Richmond, VA (US); Willie J. McKinney, Richmond, VA (US)

(21) Appl. No.: 15/713,462

(22) Filed: Sep. 22, 2017

Related U.S. Application Data
(63) Continuation of application No. 13/541,479, filed on Jul. 3, 2012, now abandoned.
Application No. 13/541,479 is a continuation of application No. PCT/US11/21593, filed on Jan. 18, 2011.
(60) Provisional application No. 61/295,555, filed on Jan. 15, 2010.

(51) Int. Cl. C12Q 1/68; G01N 33/574

(52) U.S. Cl. CPC C12Q 1/6876; C12Q 2600/172; C12Q 2600/158; G01N 2800/382; G01N 2800/323; G01N 2800/122; G01N 2800/12; G01N 2800/104; G01N 33/57423; C12Q 2600/156; C12Q 1/6883

(57) ABSTRACT

The technology provided herein relates to the SNPs identified as described herein, both singly and in combination, as well as to the use of these SNPs, and others in linkage disequilibrium with these SNPs, for diagnosis, prediction of clinical course, and/or treatment response for pulmonary disease such as COPD, development of new treatments for pulmonary disease such as COPD based upon comparison of the variant and normal versions of the gene or gene product, and development of cell-culture based and animal models for research and treatment of pulmonary disease such as COPD. The technology provided herein further relates to novel compounds, pharmaceutical compositions, and kits for use in the diagnosis, treatment, and evaluation of such disorders.

This application is a continuation of U.S. patent application Ser. No. 13/541,479, filed Jul. 3, 2012, which is a continuation of International Application No. PCT/US2011/021593, filed Jan. 18, 2011, which claims the benefit of U.S. Provisional Application No. 61/295,555 filed Jan. 15, 2010, the entirety of each of which applications is incorporated by reference herein.

### INCORPORATION OF SEQUENCE LISTING

This application contains a sequence listing submitted electronically via EFS-web, which serves as both the paper copy and the computer readable form (CRF) and consists of a file entitled “001881-8006US02_seqlist.txt”, which was created on Sep. 22, 2017, which is 274,432 bytes in size, and which is herein incorporated by reference in its entirety.

### FIELD

The field of the technology provided herein relates generally to pulmonary and related diseases and the diagnosis and prognosis thereof.

### BACKGROUND

Chronic obstructive pulmonary disease (COPD) is a complex disease characterized clinically by airflow obstruction, with cigarette smoking considered its primary environmental risk factor.

COPD is currently the fourth leading cause of chronic morbidity and mortality in the United States (National Institutes of Health and National Heart Lung and Blood Institute 2007, Am. J. Repir. Crit. Care Med. 176:532-555; Mannino and Braman 2007, Proc. Am. Thorac. Soc. 4:502-SEQ506). It is a preventable and treatable disease characterized by airflow limitation that is not fully reversible (National Institutes of Health and National Heart Lung and Blood Institute 2007). The airflow limitation results from small airway disease (obstructive bronchiolitis) and parenchymal destruction (emphysema) caused by chronic inflammation and structural changes due to repeated injury and repair (National Institutes of Health and National Heart Lung and Blood Institute 2007).

Cigarette smoking is the most important environmental risk factor for COPD (Marsh et al. 2006, Eur. Respir. J. 28:883-886; National Institutes of Health and National Heart Lung and Blood Institute 2007; Mannino and Braman 2007). It is estimated that 25% to 50% of smokers may develop COPD as defined by the Global Initiative for Chronic Obstructive Lung Disease (GOLD) spirometric criteria, (Lundbäck et al. 2003, Respir. Med. 97:115-122; Lokke et al. 2006, Thorax 61:935-939; Mannino and Braman 2007)

Lung function declines gradually across adult life, even in healthy non-smokers, and this decline accelerates with age (Camilli et al. 1987, Am. Rev. Respir. Dis. 135:794-799; Lange et al. 1989, Eur. Respir. J. 2:811-816; Lundbäck et al. 2003; Wise 2006, Am. J. Med. 119 ((10A)):S4-S11). Factors associated with lung function decline in middle-aged and older adults have been identified, primarily in cross-sectional studies (Enright et al. 1994, Chest 106:827-834; Kerstjens et al. 1996, Am. J. Repir. Crit. Care Med. 154:S266-S272). However, predictions based on cross-sectional correlates may not adequately predict longitudinal change within individuals (Knudson et al. 1983, Am. Rev. Respir. Dis. 127:725-734; Griffith et al. 2001, Am. J. Respir. Crit. Care Med. 163:61-68), and the effect of cigarette smoking on trajectories of lung function decline throughout adult life have not been widely modeled using longitudinal statistical methods.

COPD is a heterogeneous disease of complex etiology, including genetic and environmental components. Lung function is determined by the interplay of multiple underlying factors and processes. Consequently, impaired lung function in any individual may have different causes (e.g., prenatal effects, poor baseline lung function, age, and exposure to occupational toxins and cigarette smoke). Given that these risk factors are likely to act through distinct biological mechanisms, methods for discovering biomarkers associated with impaired lung function must account for this likely etiological heterogeneity. Conventional outcome measures of lung function, such as clinically based COPD case-control status and spirometric measurements, are limited in this respect. Exposure is generally not considered quantitatively, and cross-sectional measures cannot assess the trajectory of lung function decline. Conversely, longitudinal data offer the possibility of deconvoluting the etiological factors affecting lung function. The advantage lies in the structure of the data-repeated measurements of lung function and various risk factors (e.g., age, smoking exposure) collected for the same individuals over time. That data structure allows quantification of differences in susceptibility to the various causes of lung function decline across individuals.

In view of the foregoing, longitudinal data, containing repeated measurements of lung function and various risk factors, were analyzed to quantify differences underlying the susceptibility to the various causes of lung function decline. The data included four outcome measures of lung function or decline in lung function, measured spirometrically as the forced expiratory volume in 1 second (FEV₁) (Knudson et al., 1983) and were derived by fitting mixed models to longitudinal spirometric, smoking history, and demographic data obtained over the subjects' 17-year average participation period in the Lung Health Study (LHS) and General addiction Project (GAP). Conceptually, these measures represent different underlying biological processes driving lung function decline. The optimal model of the data was selected based on likelihood ratio tests, which were used to determine the significance of each fixed and random effect parameter as it was added to the model (Willet et al., 1998, Developmental Psychopathology 1998; 10:395-426). After the optimal model was identified, the outcome variables were calculated as best linear unbiased predictors (BLUPs) of the random effects, focusing on age-related decline (Age decline), pack-years-related decline (Pack-years decline), and the intensifying effects of smoking, in terms of number of cigarettes per day (CPD), on decline with age (CPD×Age decline). These BLUPs together accounted for the vast majority of individual differences in lung function decline in these subjects. In addition, Baseline Lung function (BL) was measured at subjects' entry into the study as an outcome measure as it has also been shown to vary in magnitude across individuals (Griffith et al., 2001).

There is some evidence that immune system dysregulation may be involved in the pathophysiology of COPD and that genetic differences in regulation of cigarette smoking-related inflammatory changes may influence individual disease risk.

### SUMMARY

Work described herein relates to the discovery of associations between pulmonary disease such as COPD and variations in the nucleotide sequence of nineteen chromosomal regions. Embodiments described herein provide chromosomal regions and SNPs found therein having significant novel COPD associations. As described below, some of the SNPs are in or near genes that function in biological processes such as cilia function/lung clearance, neutrophil activation, and complement regulation. The genes, intragenic regions, and identified variations in the nucleotide sequence in those regions (e.g., SNPs) associated with COPD found in each of the nineteen chromosomal regions provided herein are listed in Tables 5a, 5b, 7, 8 and/or in FIG. 8.

Based on the identification of those chromosomal regions including specific SNPs associated with pulmonary disease, such as COPD, methods are provided for detecting a predisposition to, or diagnosing the presence of, lung disease, such as COPD. Such methods comprise identifying one or more variations in a nucleotide sequence of one or more of those chromosomal regions. Variations in the nucleotide sequence of those regions, identified herein as chromosomal regions 1-19, can be correlated with a predisposition to, or the presence of, COPD in a subject.

Methods are provided for detecting a predisposition to, or diagnosing the presence of, lung disease in a subject described herein, including the use of a variety of genetic and molecular techniques to identify variations in the nucleotide sequence of chromosomal regions 1-19 in the subject. Evaluation of the nucleotide sequence to identify variation in those chromosomal regions may be conducted at the level of chromosomal DNA, or portions thereof (e.g., PER amplified gene segments). Alternatively, evaluation of the nucleotide sequence to identify variation in those regions may be conducted at the level of molecules expressed or encoded by those chromosomal regions (e.g., mRNAs or protein coding regions thereof or polypeptide/proteins encoded by those chromosomal regions).

In one embodiment, a method of detecting a predisposition to, a diagnosis of, a prognosis of, the severity of, or the response to treatment for a pulmonary disease (e.g., COPD) in a subject comprises identifying variations in the nucleotide sequence of one or more chromosomal regions selected from regions 1-19 of said subject, where the presence of one or more variations in said chromosomal regions indicates a predisposition to, or the presence of, COPD in the subject; wherein said variations in nucleotide sequence have a q-value of less than 0.5 for their association with decline in lung function.

Kits described herein can be used, for example, in performing one or more of the methods described herein. One embodiment provides for a kit comprising one or more nucleic acid probes for the identification of one or more variations in a nucleotide sequence of one or more chromosomal regions selected independently from regions 1-19. Such kits may further comprise one or more control nucleic acid molecules for said variations in said nucleotide sequence. In some embodiments, the kit comprises a means for identifying an amino acid sequence or a variation in an amino acid sequence encoded by a gene in a chromosomal region selected from regions 1-19. In one embodiment, the kit comprises an antibody that is capable of identifying an amino acid sequence encoded by a gene in a chromosomal region selected from regions 1-19. Such kits optionally comprise instructions describing the use of the kit.

In one embodiment, the present disclosure provides for compositions comprising two or more nucleic acid molecules that each comprise a nucleotide sequence complementary to different portions of chromosomal regions 1-19. In one aspect of such an embodiment, the two or more nucleic acid molecules comprise two, three, four, five, six, seven, eight, nine, ten, fifteen, nineteen or more nucleic acid molecules and said different portions of chromosomal regions 1-19 comprise portions of two, three, four, five, six, seven, eight, nine, ten, fifteen, nineteen or more different independently selected chromosomal regions.

Also provided for herein are compositions comprising two or more, three or more, four or more, five or more, or six or more nucleic acids that hybridize to different portions of chromosomal regions 1-19, each of the different portions comprising one or more variations (or at least a part of a variation) found in chromosomal regions 1-19. Also provided for herein are compositions comprising two or more, three or more, four or more, five or more, or six or more nucleic acids that hybridize to different portions of chromosomal regions 1-19.

Also described herein are pharmaceutical compositions comprising one or more gene products, active portions thereof, or variants thereof for use in the treatment of a pulmonary disease. Also provided herein are methods of using one more nucleic acid molecules encoding one or more of the gene products, an active portion(s) thereof, or variant(s) thereof for use in the treatment of pulmonary diseases such as COPD. In some embodiments, the one or more gene(s) encoding the one or more gene products are selected from the group including CLEC4A, CSMD1, DNAH3, EBF2, ELMO1, MYO5B, ENPP6, KBTBD9, MSRB3, and TSC2.

Compositions are provided comprising two or more pairs of nucleic acid molecules that may function, for instance, as primers sets for the amplification of various portions of chromosomal regions 1-19. In such embodiments, the two or more pairs of nucleic acid molecules comprise a first pair of nucleic acid molecules and a second pair of nucleic acid molecules. The first pair of nucleic acid molecules comprises (i) a first nucleic acid molecule comprising a nucleotide sequence complementary to a portion of a chromosomal region selected from chromosomal regions 1-19 and (ii) a second nucleic acid molecule comprising a nucleotide sequence complementary to the opposite strand of the chromosomal region to which said first nucleic acid is complementary. The second pair of nucleic acid molecules comprises (iii) a third nucleic acid molecule comprising a nucleotide sequence complementary to a portion of a chromosomal region selected from chromosomal regions 1-19 and (iv) a fourth nucleic acid molecule comprising a nucleotide sequence complementary to the opposite strand of the chromosomal region to which said third nucleic acid is complementary.

Also described herein are pharmaceutical compositions comprising one or more gene products, active portions thereof, or variants thereof for use in the treatment of a pulmonary disease. The genes encoding the one or more gene products can be selected from the group consisting of genes listed in Tables 5b, 6 and FIG. 3. In some embodiments, the genes encoding the one or more gene products are selected from CLEC4A, CSMD1, DNAH3, EBF2, ELMO1, MYO5B, ENPP6, KBTBD9, MSRB3, and TSC2. One embodiment provides for the use of agonists and antagonists of the activity of one or more of the gene products listed in Tables 5, 6 and FIG. 3 for use in the treatment of pulmonary diseases such as COPD. Another embodiment of the technology provided for herein is directed to a method of using agonists and antagonists of the activity of one or more of the gene products of the genes in chromosomal regions 1-19. In one such embodiment, agonists and antagonists alter the activity of one or more products of genes selected from the group consisting of CLEC4A, CSMD1, DNAH3, EBF2, ELMO1, MYO5B, ENPP6 KBTBD9, MSRB3, and TSC2. Such pharmaceutical compositions may be used in the treatment of pulmonary diseases such as COPD. Agonists and antagonists can include not only small molecule inhibitors of those genes or inhibitory RNA molecules (e.g., antisense or siRNA), but also antibodies or antigen binding fragments thereof. Such antibodies include, but are not limited to, polyclonal antibodies (e.g., monospecific polyclonal antibodies), monoclonal antibodies, humanized antibodies, or fragments thereof such as scFv, Fab, Fab′, a F(ab′)₂, Fv, or disulfide linked Fv fragments.

The techniques provided herein permit the use of genetic variations, such as the SNPs identified as described herein, both singly or in combination with other variations in linkage disequilibrium (LD) with those SNPs, for the diagnosis, prediction of clinical course (prognosis), and/or assessment of treatment effect/patient response for pulmonary disease such as COPD. Additional uses include development of new treatments for pulmonary disease such as COPD, based upon comparison of the variant and normal versions of the gene or gene product, and development of cell culture-based and animal models for research and treatment of pulmonary disease such as COPD.

Another embodiment of the present technology provides a method of detecting a predisposition to, a diagnosis of, a prognosis of, the severity of, or the response to treatment for a pulmonary disease (e.g., COPD) in a mammal, comprising assaying the product of at least one gene selected from the group consisting of CLEC4A, CSMD1, DNAH3, EBF2, ELMO1, MYO5B, ENPP6, KBTBD9, MSRB3, and TSC2.

Assaying a gene may be conducted by determining the expression of a nucleic acid product (e.g., an mRNA) produced by the gene. Where nucleic acid levels are to be determined, a variety of techniques including quantitative PCR, Southern blotting or Northern blotting may be employed. Alternatively, assaying a gene may be conducted either by assessing the level of the protein produced, or by examining the biological activity of the protein product. The level of protein present in a sample may be determined by methods including, but not limited to, immunological methods (e.g., ELISA or Western blot) and also by the activity of the protein in either biological or enzymatic assays. As SNPs within protein coding sequences may affect the biological activity or stability of proteins due to alterations in the protein sequence, assaying a combination of protein level and its biological activity, or the level of gene expression (e.g., mRNA production) and the protein's biological activity may be desirable when assaying a gene product involves assaying a protein.

In some embodiments, a method of predicting a predisposition to, a diagnosis of, a prognosis of, the severity of, or the response to treatment for a pulmonary disease in an individual (a subject) involves obtaining a sample from the individual, wherein the biological sample contains, or is expected to contain, all or a portion of the gene product of the genes listed in Tables 5b, 6 and/or FIG. 3. Alternatively, such methods may employ a sample that comprises all or a portion of any protein or peptide encoded by genes in linkage disequilibrium found in each of the nineteen chromosomal regions provided herein (see e.g., Tables 5a, 5b, 7, 8 and/or in FIG. 8). Where samples comprise proteins or peptides, such methods comprise determining the amino acid(s) present at one or more positions of the proteins/peptide encoded by the regions in linkage disequilibrium. In some embodiments, the presence of one or more amino acid sequences is indicative of the presence of one or more of the SNPs whose presence is indicative of a pulmonary disease. In one version of such embodiments, the pulmonary disease is COPD.

In one embodiment, the present disclosure provides nucleic acid molecules that can be inserted in an expression vector to produce a variant protein in a host cell. Thus, the present disclosure provides for vectors comprising a SNP-containing nucleic acid molecule(s) that can be functionally linked to a promoter, genetically engineered host cells containing the vector, and methods for expressing a recombinant variant protein including the use of host cells containing such vectors. The host cells, SNP-containing nucleic acid molecules and/or variant proteins can also be used as targets in a method for screening and identifying therapeutic agents or pharmaceutical compounds useful in the treatment of pulmonary disease and related pathologies.

Also provided herein are methods of using one or more nucleic acid molecules encoding one or more of the gene products, an active portion(s) thereof, or variant(s) thereof, for use in the treatment of pulmonary diseases such as COPD. In some embodiments, the one or more genes encoding the one or more gene products are selected from the group including CLEC4A, CSMD1, DNAH3, EBF2, ELMO1, MYO5B, ENPP6, KBTBD9, MSRB3, and TSC2.

Another aspect of the technology described herein is kits, which can be used, for example, in performing one or more of the methods described herein. One embodiment provides for a kit comprising one or more nucleic acid probes, wherein the probes allow the identification of either a nucleic acid having a nucleotide sequence of a SNP associated with pulmonary disease (e.g., COPD) found in one of the nineteen chromosomal regions provided herein (see Tables 5a, 5b, 7, 8 and/or in FIG. 8), or a control nucleic acid, and a pamphlet describing the use of the kit in the diagnosis, prognosis, and/or severity prediction of a pulmonary disease (e.g., COPD) or in determining the response of a subject to a treatment for a pulmonary disease. In some embodiments, the kits comprise a nucleic acid probe, wherein the probe allows measuring an allele for a SNP listed in Tables 5a, 5b, 7, 8 and/or in FIG. 8, a control, and a pamphlet describing the use of the kit in relation to pulmonary disease (e.g., COPD). Controls for such kits can be nucleic acids. In some embodiments, the control is selected from the group consisting of homozygous reference genotype, homozygous variant genotype, heterozygous genotype, and combinations thereof for the particular SNP identified by the probe. In some embodiments, the control is a single base extension and fluorescence resonance energy transfer (SBE-FRET) primer. In some embodiments, the probe binds to a region adjacent to the SNP.

In some embodiments, the kit comprises a means suitable for identifying an amino acid sequence selected from the group consisting of amino acid sequences encoded by nucleic acids bearing a variation in LD with a SNP listed in Tables 5a, 5b, 7, 8 and/or in FIG. 8 and an amino acid sequence that is encoded by an alternate allele of a SNP listed in Tables 5a, 5b, 7, 8 and/or in FIG. 8. Such kits may also comprise a control, and a pamphlet describing the use of the kit in relation to COPD diagnosis or prognosis. In some embodiments, the means for identifying the amino acid sequence comprises an antibody that is capable of binding a protein, polypeptide, or peptide having the sequence of interest. In some embodiments, the control comprises a control antibody. In some embodiments, the control comprises a protein or polypeptide having an amino acid sequence that is produced by an alternate allele of a SNP listed in Tables 5a, 5b, 7, 8 and/or in FIG. 8 or in LD with listed SNPs.

In some embodiments of the kits provided herein, the control is an assay standard, such as a sample of the protein being assayed (e.g., a protein produced by a gene associated with an SNP such as CLEC4A, CSMD1, DNAH3, EBF2, ELMO1, MYO5B, ENPP6, KBTBD9, MSRB3, and TSC2) or a nucleic acid (e.g., DNA or RNA) bearing one of the SNPs listed in Tables 5a, 5b, 7, 8 and/or in FIG. 8. In some embodiments of the kits provided herein, the pamphlet includes the description of use of the kit in relation to COPD diagnosis or prognosis and includes instructions for analyzing results obtained using the kit.

In some embodiments, the kits provided herein comprise one or more chips or high-density arrays that contain many individual regions bearing a binding partner, such as a nucleic acid, for determining the presence or measuring the quantity of nucleic acid molecules present in a sample. Where assays are conducted using arrays of nucleic acids as molecular probes, the array can comprise a SNP listed in Tables 5a, 5b, 7, 8 and/or in FIG. 8. Such chips permit the rapid detection and/or measurement of polymorphisms and/or mutations, providing a convenient means for the determination of those individuals at high or at low risk of developing COPD. The detection of specific polymorphisms in specific patients will allow highly specific and individualized treatment strategies to be devised for each patient to prevent or attenuate COPD.

Other embodiments are directed to devices. In one embodiment, the device comprises a test surface having a plurality of locations, wherein one or more of said locations comprise an antibody that binds to the product of a gene associated with a SNP listed in Tables 5a, 5b, 7, and 8 and/or in FIG. 8. In another embodiment, the device comprises a test surface having a plurality of locations, wherein one or more of said locations comprise one or more nucleic acids having nucleotide sequences complementary to at least a portion of the sequence found at one or more of the SNP locations listed in Tables 5a, 5b, 7, 8 and/or in FIG. 8.

The various embodiments described herein can be complementary and can be combined or used together in a manner understood by the skilled person in view of the teachings contained herein.

### BRIEF DESCRIPTION OF THE DRAWINGS

FIG. 1 is a plot showing association evidence and linkage disequilibrium (LD) within a portion of the CSMD1 gene markers having a p-value ≦0.0005; vertical lines above SNP names are −log₁₀ of the p-values for all markers tested in the region; LD blocks are defined using solid spline of LD.

FIGS. 2A-2D illustrate a plot of SNPs showing linkage disequilibrium (LD) within the MYO5B gene in Region 19. FIG. 2A shows the overall layout of the MYO5B gene and the ACAA2 gene for acetyl-coenzyme A acyltransferase. Expanded segments of the MYO5B gene showing SNP locations are shown in FIGS. 2B, 2C and 2D. The vertical lines above SNP names are the −log₁₀ of the p-values for all markers tested in the region; LD blocks were defined using solid spline of LD.

FIG. 3 is a schematic illustrating the neutrophil as a unifying target.

FIG. 4 shows a QQ plot of Pack-years decline BLUP (produced using 10 sets of random p-values from a uniform distribution).

FIG. 5 is a QQ plot showing Age decline BLUP.

FIG. 6 is a QQ plot showing CPD×Age decline BLUP.

FIG. 7 is a QQ plot showing Baseline lung function BLUP.

FIG. 8 is a table showing regions 1-19 as defined by chromosomal markers recited therein.

### DETAILED DESCRIPTION

As demonstrated herein, analysis of polymorphisms in the genes and regions identified herein leads to an ability to identify subjects that may have a predisposition to, or heightened risk of, developing a pulmonary disease, and to predict whether the subject may benefit from monitoring, prophylactic treatment, and/or treatment. Analysis of polymorphisms in the genes and regions identified herein also leads to an ability to diagnose a pulmonary disease, to predict the development of a pulmonary disease, to determine the probability of its development, and to predict its ultimate severity. Such predictions may be made based upon an analysis either of the polymorphisms alone, or in conjunction with other clinically relevant information, such as continued smoke exposure, or the presence of biochemical markers, such as nitrite levels, catalase activity and lipid peroxidation in plasma of an individual. See e.g., U.S. Application 20060177830. The SNPs disclosed herein may contribute to pulmonary disease and related pathologies in an individual in a variety of ways. Some SNPs occur within a protein coding sequence and thus, may directly contribute to disease phenotype. Other polymorphisms may occur in noncoding regions but may exert phenotypic effects indirectly, such as, for example, by influencing replication, transcription, translation, or other regulation of a gene. An individual SNP may also affect more than one phenotypic trait. Alternatively, a single phenotypic trait may be affected by multiple SNPs in the same or different genes.

### 1.0 Genome Wide Association Analysis and Identification of Chromosomal Regions

COPD is predicted to become the third leading cause of death worldwide by 2020 (Mannino & Braman 2007), and cigarette smoking is widely recognized as its primary environmental causative factor. The pulmonary component of COPD is primarily characterized by airway inflammation with incompletely reversible, usually progressive, airflow obstruction (Rabe et al. 2007, Am J Respir. Crit Care Med., vol. 176, no. 6, pp. 532-555; Barnes et al. 2003, Eur Respir J, 22:672-688; Barnes 2003, Annu Rev Med 54:113-129). The identified pathophysiologic mechanisms of COPD include an imbalance between protease and anti-protease activity in the lung, dysregulation of anti-oxidant activity and chronic abnormal inflammatory response to long-term exposure to noxious gases or particles leading to the destruction of the lung alveoli and connective tissue (Rabe et al. 2007, Barnes et al. 2003, Barnes 2003). However, COPD may be best characterized as a syndrome associated with significant systemic effects that are attributed to low-grade, chronic systemic inflammation (Agusti et al. 2003, Euro. Resp. J. 21.2: 347-60; Rahman et al. 1996, Amer. J. of Resp. and Crit. Care Med. 154.4 Pt I (1996): 1055-60; Agusti & Soriano 2008, J. of Chronic Obstructive Pulmonary Disease 5: 133-38; Fabbri & Rabe 2007, Lancet, 370 (2007): 797-99). Although spirometric parameters are the traditional gold standard diagnostic and prognostic markers for COPD, it has become clear that they do not adequately represent all of its respiratory and systemic aspects (Marin et al. 2009, Respir Med 103:373-8; Celli 2006, Proceedings of the Amer. Thoracic Society 3:461-465). FEV₁ correlates poorly with the degree of dyspnea, and the change in FEV₁ does not reflect the rate of decline in health status (Celli et al. 2004, The New England J. of Med. 350:1005-1012; Celli 2006; Burge et al. 2000, British Medical J. 320:1297-1303). Other factors, such as emphysema and hyperinflation (Casanova et al. 2005, Amer. J. of Resp. and Crit. Care Med. 171:591-597), malnutrition (Schols et al. 1998, Amer. J. of Resp. and Crit. Care Med. 157:1791-1797), peripheral muscle dysfunction (Maltais et al. 2000, Clinics in Chest Med. 21:665-677), and dyspnea (Nishimura et al. 2002, Chest 121:1434-1440), are independent predictors of outcome. In fact, the multifactorial BODE index that includes body mass index (B), degree of airflow obstruction (O), dyspnea score (D), and exercise endurance (E), was a better predictor of mortality than FEV₁ alone (Celli et al. 2004). The PBMC gene expression profile alone or in combination with clinical markers such as the BODE index components and/or lung parenchymal or airway changes on chest CT scans (Omori et al. 2006, Respirology 11:205-210) may be more predictive of the (early) presence, activity, and progression of the multi-component syndrome that is COPD compared to the clinical parameters alone.

The incompletely reversible airflow limitation observed in COPD results from small airway disease (obstructive bronchiolitis) and parenchymal destruction (emphysema). These pathologic changes are the result of an abnormal inflammatory response to long-term exposure to noxious gases or particles, with structural changes due to repeated injury and repair (Rabe et al. 2007). The mechanisms of the enhanced inflammation that characterizes COPD involve both innate and adaptive immunity in response initially to inhalation of particles and gases (MacNee 2001, Euro. J. of Pharmacology, vol. 429, pp. 195-207). Several studies have demonstrated differences in markers of inflammation and immune response, such as a correlation between the number of CD8 cytotoxic T lymphocytes and the degree of airflow limitation in COPD (Curtis, et al. 2007, Proc. of the Amer. Thoracic Soc., vol. 4, no. 7, pp. 512-521). The response to oxidative stress is considered an important factor in the pathogenesis of COPD (MacNee 2005, Proc. of the Amer. Thoracic Soc., vol. 2, no. 1, pp. 50-60), while protease-antiprotease imbalance is thought to be associated with emphysema (Baraldo et al. 2007, Chest, vol. 132, no. 6, pp. 1733-1740). However, while inflammation and other factors are clearly involved in the molecular pathogenesis of COPD, the precise etiological mechanisms remain to be fully characterized.

Novel genetic associations with lung functions that decline as a function of increasing cigarette smoking, after controlling for the effects of age and baseline lung function, are provided herein. As described herein, a genome-wide association study (GWAS) investigation of COPD was performed. Over 550,000 genetic markers were genotyped and tested for association in a sample of 192 adult cigarette smokers with COPD who were followed longitudinally over 17 years and in 197 age- and gender-matched control subjects (smokers and never-smokers without COPD). The outcomes for the association analyses were four spirometry-based indices that deconvoluted the major biological processes driving lung function decline, as well as the conventional dichotomous case-control categorization. The four spirometry-based outcome variables were calculated as best linear unbiased predictors (BLUPs) of lung function decline and focused on age-related decline (Age decline), pack-years-related decline (Pack-years decline), the intensifying effects of smoking, in terms of number of cigarettes per day (CPD), on decline with age (CPD×Age decline), and Baseline lung function (BL).

The results from the GWAS were examined in two contexts. In one context, results were examined to identify chromosomal regions where variations in the nucleotide sequence (e.g., the introduction of SNPs, deletions, insertions, etc.) were found to be associated with a decline in lung function. Second, the results were examined in the context of genes associated with the identified chromosome regions to identify biological/biochemical pathways whose impairment may be associated with lung disease and which are predictive of a predisposition to or the presence of pulmonary diseases like COPD. Such pathways may be identified by the presence of one or more genes in the identified chromosomal regions associated with recognized biological/biochemical pathways. Once identified, the pathways may be of further use in defining methods of diagnosis, prognosis, severity prediction, and treatment of pulmonary disease such as COPD.

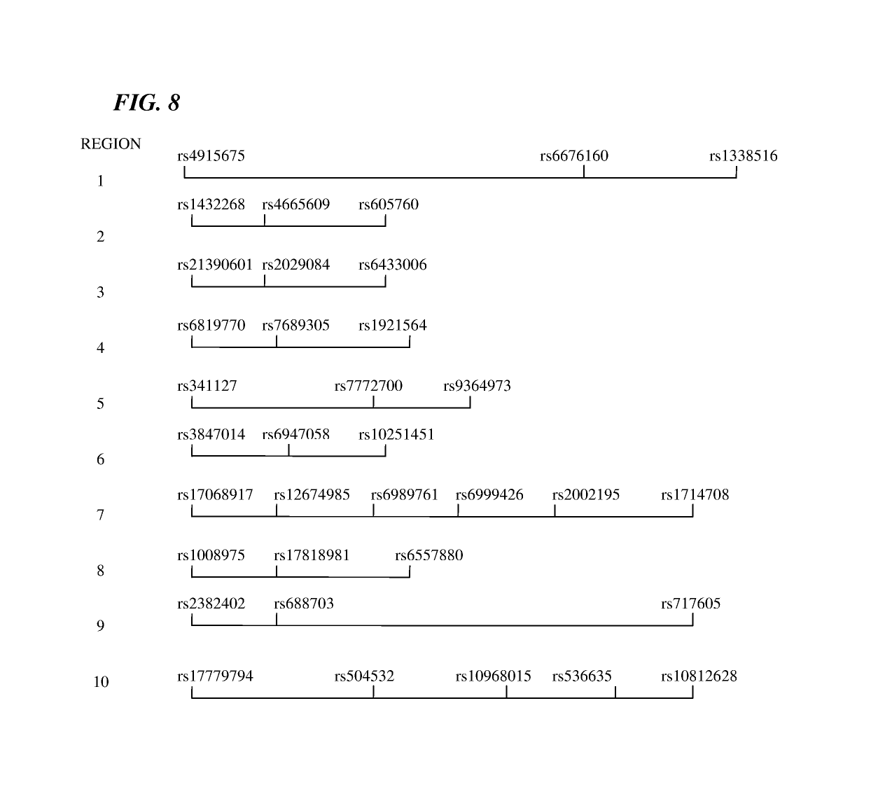

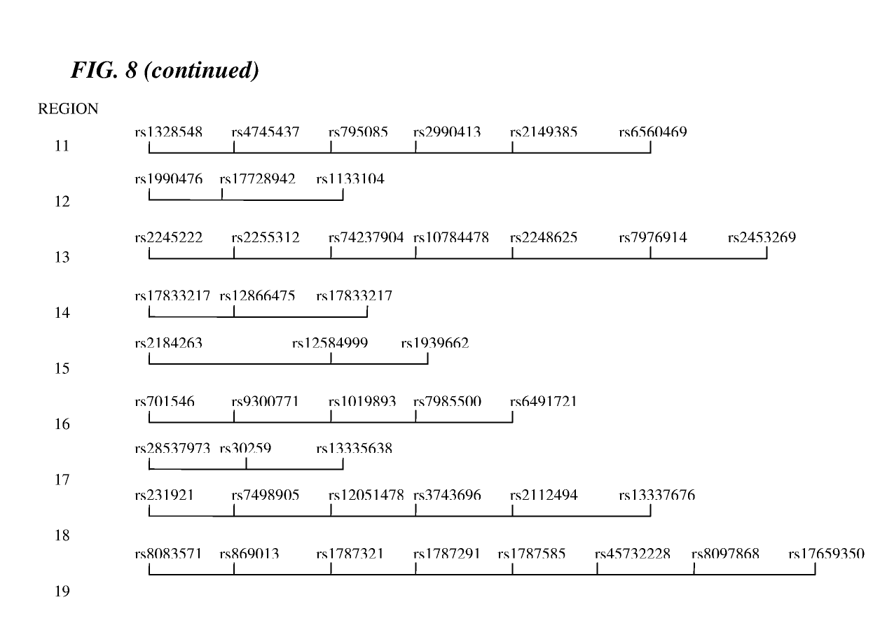

The present disclosure identifies nineteen chromosomal regions having significant associations with pulmonary disease such as COPD. Those regions include one or more genes and identified polymorphisms (e.g., SNPs). As described below, some of the chromosomal regions include SNPs that are in, or that are near, genes that function in biological processes such as cilia function/lung clearance, neutrophil activation, and complement regulation. The genes, intragenic regions, and SNPs associated with COPD found in each of the nineteen chromosomal regions provided herein are listed in Tables 5a, 5b, 7, 8 and/or in FIG. 8. The variations (e.g., SNPs) identified in those regions may be used in any combination in any of the methods recited herein. In one embodiment, the variations are variations in regions 1-19. In another embodiment, the variations are variations in regions 1-18. In still another embodiment, the variations are variations in region 19.

Based on the identification of those chromosomal regions, the present disclosure provides methods of detecting a predisposition to, a diagnosis of, a prognosis of, the severity of, or the response to treatment for a pulmonary disease (e.g., COPD), in a subject. In one embodiment, the methods comprise identifying in a subject's chromosomes one or more variations in a nucleotide sequence of one or more of the nineteen chromosomal regions identified herein. Variations in those nucleotide sequences can be correlated with a predisposition to, a diagnosis of, a prognosis of, the severity of, or the response to treatment for a pulmonary disease in a subject.

Biological processes identified as over-represented in the set of lung disease (e.g., COPD) predictor genes present in the nineteen identified chromosomal regions include: regulation of apoptosis, regulation of cell growth, macromolecule (protein and RNA) transport, post-translational protein modification, cellular defense response, inflammatory response and RNA processing. Major pathways identified include apoptosis, p38/MAPK signaling, focal adhesion, and leukocyte transendothelial migration. Changes in these biological processes and pathways may reflect the changes in activation, differentiation and cellular composition of the samples analyzed. The identification of leukocyte transendothelial migration seems to be an important change in this cell population due to the fact that COPD is characterized by leukocyte infiltration in the lung parenchyma (Panina et al. 2006). It is possible that differences in expression of these genes may result in a predisposition of leukocyte subpopulations to infiltrate the lung tissue, and perhaps other tissues. This observation is supported by previously reported changes in chemotaxis and extracellular proteolysis in neutrophils isolated from the blood of subjects with COPD (Burnett et al. 1987).

### 2.0 Identification of Variations in Chromosomal Regions

2.1 Variations and their Identification.

As used herein “variations” in a nucleotide sequence refer to differences in a nucleotide sequence in an individual relative to the sequence of nucleic acid molecules appearing in a control sequence (e.g., the sequence of chromosomal DNA for dominant allele or of a control subject) or in the larger population (e.g., the difference(s) in the sequences of chromosomal DNA giving rise to different alleles in a population of control subjects). Variations include, but are not limited to: SNPs; deletions; insertions (e.g., di-, tri-, or tetra-nucleotide repeats); variable number tandem repeats (VNTR); short tandem repeat/microsatellites; copy number variants; amplifications (e.g., duplications); translocations; transversion (the substitution of a purine for a pyrimidine); and transitions (exchanging of purines or pyrimidines present in a sequence i.e., exchanging purines A H G, or pyrimidines C A/T). The sequences at any given chromosomal location, including the prevalence of any particular base at any location may be established by any means known in the art including accessing databases (e.g., human genomic databases at the NCBI)

Variations in the nucleotide sequences found in a subject's genome (e.g., the nineteen chromosomal regions described herein) can be identified by analysis of the chromosomal material or copies of that material (e.g., PCR amplified copies of one or more portions of a subjects chromosomal DNA) using any method known in the art, including but not limited to those described below.

As used herein, a Single Nucleotide Polymorphism (SNP) is a specific position within the reference human genome that may vary between the four possible nucleotides between individuals. The different possible nucleotides are referred to as alleles.

In addition to the analysis of chromosomal material for the identification of variations in the nucleotide sequence of chromosomal regions, gene products expressed by genes located in the chromosomal regions can be analyzed (e.g. mRNA or cDNA copies thereof). It is also possible to examine proteins and polypeptides produced by genes within the chromosomal regions to identify variations in the nucleotide sequence of the chromosomal region.

Protein or nucleic acid sequence identifiers provided herein uniquely identify nucleic acid and/or protein sequence(s), (e.g., an NCBI accession number/version and/or NCBI “GI” Number). Those identifiers and the coinciding sequence(s) are publicly available, for example, at the United States National Center for Biotechnology Information (NCBI, U.S. National Library of Medicine, 800 Rockville Pike, Bethesda, Md., 20894 USA) or on the world wide web at www.ncbi.nlm.nih.gov. Where an NCBI accession number or GI number is provided for only one or two of the chromosomal sequence(s), protein sequence(s) or a nucleic acid sequence(s) encoding a protein produced by a gene indicated herein (e.g., a cDNA sequence), the sequence(s) for those nucleic acids and/or proteins not provided are also available in the NCBI database and considered part of this disclosure. Where any accession number does not recite a specific version, the version is taken to be the most recent version of the sequence associated with that accession number at the time the earliest priority document for the present application was filed.

2.2 Analysis of Nucleic Acids to Identify Variations in Chromosomal Regions

Any Method Known in the Art May be Used to Identify Variations in the Nucleotide Sequence of a subject's chromosomal DNA: including, but not limited to: sequencing, single stranded cleavage, hybridization (such as to arrays or individual nucleic acid probes), differential hybridization between the variant and a wild type sequence, single base extension, allele specific cleavage by restriction enzymes, oligonucleotide ligation assay (OLA), mass spectroscopy, and Polymerase Chain Reaction (PCR) based methods, such as amplification with allele specific primers. Nucleic acid probes used in any of those methods may be detectably labeled, such as with radioisotopes or fluorescent tags.

As used herein, a “primer” or “probe” is a nucleic acid molecule that typically comprises at least about 8, 10, 12, 14, 16, 17, 18, 19, 20, 21, 22, 23, 24, 25, 26, 27, 28, 29, or 30 nucleotides complementary to the nucleic acid sequence it is targeted against (e.g., a portion of chromosomal regions 1-19). Primers and probes may also contain nucleotide sequences in addition to the region complementary to the target sequence meaning their total length may be significantly longer than the region complementary to the target sequence. Depending on the type of assay in which it is employed, the complementary region of a probe will generally be less than 40, 50, 60, 65, 75, 100, 150, 200, or 250 nucleotides in length; however, the complementary portion of a probe may be as long as the target sequence to be detected. Primers, which are to be extended by the action of a polymerase, such as primers for nucleic acid amplification, typically comprise more than about 12 or 15 and less than about 30 nucleotides complementary to the target sequence. Like probes, primers can contain sequences in addition to the portion complementary to the target sequence, and thus may be longer than the 30 nucleotides. In some embodiments, primers or probes comprise regions complementary to the target sequence that is in a range selected from: about 16 to about 32 nucleotides, about 18 to about 28, and about 18 to about 26 nucleotides. In other embodiments, such as where probes are affixed to a substrate in a nucleic acid array, the probes can be longer, such as about 30 to about 60, 50 to about 75, 70 to about 90, or about 100 or more nucleotides in length. In still other embodiments, primers can be as long as the length of the target sequence minus one nucleotide.

A number of considerations must be taken into account when designing probes and primers including, but not limited to, the length of the primer or probe, a GC content within a range suitable for hybridization, a lack of predicted secondary structure, and the stringency of the conditions under which the hybridization between the probe or primer and the target sequence is to be performed. A skilled artisan will recognize that other factors, including the nature of the sequences surrounding a variation where a probe or primer may need to hybridize, must also be taken into consideration.

Where hybridization is used, a nucleic acid probe typically hybridizes to a target nucleic acid containing the sequence variation (e.g., SNP) by complementary base-pairing in a sequence specific manner, and discriminates the target variant sequence from other nucleic acid sequences.

In one aspect, one or more probes are employed that can differentiate between nucleic acids having a specific variation (e.g., a specific allele such as SNP) and the wild type sequence at the location of the specific variation. In an embodiment, the specific variations are selected from two or more of the SNPs recited in FIG. 8. In other embodiments, the specific variations are selected from the SNPs recited in Tables 5a or 5b.

Variations may also be detected employing a nucleic acid amplification primer (e.g., a PCR primer) that acts as an initiation point for nucleotide extension at the point of or in the variation, so that amplification will only be effective where the primer matches the variant sequence (or wild type for the control).

Where variations in nucleic acid sequences are identified using allele specific primers or probes, the design of each allele-specific primer or probe depends on variables such as the precise composition of the nucleotide sequences flanking the variation, the length of the primer or probe, a GC content within a range suitable for hybridization, lack of predicted secondary structure and the stringency of the condition under which the hybridization between the probe or primer and the target sequence is performed.

Higher stringency conditions utilize buffers with lower ionic strength and/or a higher reaction temperature. Lower stringency conditions utilize buffers with higher ionic strength and/or a lower reaction temperature. By way of example, and not limitation, one set of conditions for high stringency hybridization of allele-specific probe is: prehybridized with a solution containing 5× standard saline phosphate EDTA (5×SSPE, 50 mM NaH₂PO₄, pH 7.7, containing 0.9 M NaCl and 5 mM EDTA), 0.5% SDS) at 55° C. followed by incubation with the probe under the same conditions, followed by washing with a solution containing 2×SSPE, and 0.1% SDS at 55° C. or room temperature (about 18-24° C.).

Moderate stringency hybridization conditions (e.g., for allele-specific primer extension reactions) may utilize a solution containing about 50 mM KCl at about 46° C. Alternatively, the incubation may be conducted at an elevated temperature, such as 60° C. In another embodiment, a moderately stringent hybridization condition suitable for oligonucleotide ligation assay (OLA) reactions, wherein two probes are ligated if they are completely complementary to the target sequence, may utilize a solution of about 100 mM KCl at a temperature of 46° C.

In hybridization-based assays, allele-specific probes can be designed that hybridize to a segment of target DNA having a wild-type sequence or the sequence of a variation (e.g., alternative SNP alleles/nucleotides). Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP so that the SNP site aligns anywhere along the sequence of the probe, the probe is preferably designed to hybridize to a segment of the target sequence such that the location of the SNP aligns with a central portion of the probe (e.g., a position within the probe that is at least three nucleotides from either end of the probe). Such a probe design generally achieves good discrimination in hybridization between different allelic forms.

In an embodiment, a probe or primer may be designed to hybridize to a segment of target DNA such that the variation aligns with either the 5′ most end or the 3′ most end of the probe or primer. In an embodiment which is particularly suitable for use in an oligonucleotide ligation assay (see e.g., U.S. Pat. No. 4,988,617), the 3′ most nucleotide of the probe aligns with the SNP position in the target sequence.

Synthetic nucleic acids (e.g., Peptide Nucleic Acids, PNA) may also be used to detect variation in a nucleic acid sequence. In one embodiment, a variation such as a SNP is detected with a reagent such as a PNA oligomer, or a combination of DNA, RNA and/or a PNA, that hybridizes to a segment of a target nucleic acid molecule containing a sequence variation. In an embodiment, those variations are the SNPs identified in Table 5a, 5b, 7, 8 and/or FIG. 8.

In an embodiment, multiple detection reagents, such as probes and/or primers, may be prepared and/or employed in one or more formats. For example, multiple detection reagents may be affixed to a solid support (e.g., arrays or beads) or supplied in solution (e.g., probe/primer sets for PCR, RT-PCR, TaqMan assays, OLA assays, or primer-extension reactions). Multiple probes or primers (e.g., about 2, 3, 4, 5, 6, 8, 9, 10 or more probes and/or primers) in any of those formats may be prepared in the form of kits, which optionally contain instructions on their use in detecting sequence variations.

Those skilled in the art will understand that nucleic acid molecules may be double-stranded molecules and that reference to a particular site on one strand refers, as well, to the corresponding site on a complementary strand. In defining the position of a variation such as a SNP, a reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of nucleic acid molecule. Probes and primers may be designed to hybridize to either strand and the genotyping methods disclosed herein may generally target either strand. Primers may be designed to amplify any of chromosomal regions 1-19 identified herein or parts thereof.

2.3 Analysis of Polypeptides and/or Proteins to Identify Variations in Chromosomal Regions

Variations in the nucleotide sequence of one or more of a subject's chromosomal regions can be identified by examining the protein or polypeptide gene products encoded by the chromosomal regions. In one embodiment, variant polypeptides or variant proteins that differ from the “wild type” proteins encoded by the genes of the nineteen chromosomal regions associated with COPD and other lung disease may be used to identify the presence of variations in the nucleotide sequence of a subject's chromosomal DNA. Variant polypeptides and proteins include, but are not limited to, proteins or polypeptides having: a single or multiple amino acid difference, truncations, additions, insertions, or deletions, arising from the variations in the nucleotide sequences encoding them relative to the wild type polypeptide/protein (e.g., SNPs may introduce missense mutations, nonsense mutations, or read-through mutations that remove a stop codon). For the purpose of this disclosure the wild type proteins/polypeptides are considered to be the polypeptides and proteins encoded by the sequences of the nineteen chromosomal regions identified in this disclosure. Where variations in a subject's chromosomal DNA do not arise in the sequences encoding gene products, the variations may still alter the level of expression of the polypeptide or protein encoded by the gene.

In an embodiment, the variant polypeptides or proteins are selected from the proteins CLEC4A, CSMD1, DNAH3, EBF2, ELMO1, ENPP6, KBTBD9, MSRB3, MYO5B, ENPP6 and TSC2. In another embodiment, the variant polypeptides or proteins are selected from CSMD1, MYO5B, and DNAH3. In another embodiment, the variant polypeptides or proteins are selected from CLEC4A, EBF2, ELMO1, and TSC2.

Alterations in polypeptides or proteins (including CLEC4A, CSMD1, DNAH3, EBF2, ELMO1, ENPP6, KBTBD9, MSRB3, MYO5B, ENPP6 and TSC2) may be identified by any means known in the art, including but not limited to: antibodies specific to changes in the amino acid sequence caused by a variation, the size of the polypeptides/proteins observed (e.g., where insertions, deletions, non-sense or read through mutations have occurred), and mass spectroscopy of the polypeptides/proteins or fragments thereof (e.g., tryptic digests). In addition to the foregoing, where variations in nucleotide sequences alter a biochemical activity (e.g., enzymatic activity or binding to ligand), assays of the activity may be used to assess the presence of variations in the nucleotide sequence of a chromosomal region.

Where the level of polypeptide/protein expression is altered in a subject, changes in the level of expression may be identified in any suitable assay including, but not limited to immunoassays or biochemical assays such as enzymatic assays. In an embodiment, activity assays of ENPP6 or MSRB3 are used to identify variations in the nucleotide sequence encoding those proteins.

### 3.0 Assessment of Genetic Predispositions to Pulmonary Disease and Diagnosis of Pulmonary Disease in Subjects

It is possible to provide an estimate of a subject's predisposition to, diagnosis of, or prognosis (e.g., expected severity) of pulmonary disease (e.g., COPD) by identifying variations in the nucleotide sequence of one or more of the nineteen chromosomal regions identified herein. As described herein, variations in those chromosomal regions, including specific SNPs described in any of Tables 5a, 5b, 7 and/or 8, can be associated with an increased risk of having or developing pulmonary disease and related pathologies. Thus, where certain sequence variations (e.g., SNPs) can be identified in a subject's chromosomal DNA, they may be employed to determine whether an individual possesses an increased risk of developing pulmonary disease such as COPD or a related disorder (i.e., they have a predisposition to pulmonary disease). The presence of those sequence variations can also be used in the diagnosis of lung disease, such as COPD, or to provide a prognosis for the COPD.

In one embodiment, a method of detecting/determining a predisposition to, a diagnosis of, a prognosis of, the severity of, or the response to treatment for a pulmonary disease (e.g., COPD) in a subject comprises identifying variations in the nucleotide sequence of one or more chromosomal regions selected from regions 1-19 of said subject, where the presence of one or more variations in said chromosomal regions are indicative of a predisposition to, or the presence of, COPD in the subject.

Variations in chromosomal regions may be the variations identified in Tables 5a, 5b, 7, 8 and/or in FIG. 8, variations in linkage disequilibrium with those variations, or variations within regions 1-19 as set forth in Tables 5a, 5b and/or in FIG. 8 that show a statistically significant association with pulmonary diseases such as COPD. In other embodiments, variations found in chromosomal regions may be statistically significant variations that fall within 500, 1,000, 2,000 or 2,500 bases of any statistically significant SNP identified herein. As such, the chromosomal variations with statistically significant associations may fall outside of the nineteen chromosomal regions identified in FIG. 8. In another embodiment, the chromosomal variation may be found in the regions flanking any of the chromosomal regions defined herein at a distance that may be expressed as a percentage of the length of the chromosomal region. Thus, variations with statistically significant associations may be those found in the nineteen chromosomal regions including a sequences within 1, 2, 5, 7 or 10% of the region's length. Statistically significant associations may be shown where the variations have a q-value of less than 0.5 or a p-value of 0.05, 0.02, 0.01, 0.005 or less (depending on the stringency desired) for their association lung function or a decline in lung function.

In one embodiment, chromosomal variations that are associated with pulmonary diseases at a statistically significant level include those variations found within any of regions 1-19 and those within 2,500 base pairs of any SNP within those regions identified as having a statistically significant association with a pulmonary disease described herein. In another embodiment, chromosomal variations that are associated with pulmonary diseases at a statistically significant level include those variations found within any of regions 1-19, and those statistically significant variations within a distance that is equal to 10% of the length (as measured in base pairs) of the individual chromosomal regions. In either case, statistically significant associations may be shown where the variations have a q-value of less than 0.5 or a p-value of 0.05, 0.02, 0.01, 0.005 or less (depending on the stringency desired) for their association with lung function or its decline (e.g., % predicted FV₁, % predicted FVC, or the ratio of FEV1/FVC).

Unless stated otherwise, the terms “diagnose”, “diagnosing”, “diagnosis”, and “diagnostics” used herein include, but are not limited to, any of the following: detection of pulmonary disease and/or a related pathology that a subject may presently have; determining a particular type or subclass of pulmonary disease in a subject known to have pulmonary disease; confirming or reinforcing a previously made diagnosis of pulmonary disease; pharmacogenomic evaluation of a subject to determine which therapeutic strategy the subject is most likely to positively respond to or to predict whether a patient is likely to respond to a particular treatment; predicting whether a patient is likely to experience negative effects from a particular treatment or therapeutic compound; and evaluating the future prognosis of an individual having a pulmonary disease. Such diagnostic uses can be based on the SNPs individually or a unique combination of SNPs. In addition to use as diagnostics the SNPs, individually or as a combination of SNPs, may also be used to stratify enrollment in clinical research trials of therapeutics or prophylaxis/treatment modalities to enrich for a response with a smaller sample size (i.e., smaller number of subjects).

In one embodiment, an individual or a population of individuals may be considered as not having pulmonary disease (lung disease) or impaired lung function when they do not exhibit clinically relevant signs, symptoms, and/or measures of lung disease. Thus, in various aspects, an individual or a population of individuals may be considered as not having pulmonary disease (e.g., chronic obstructive pulmonary disease, chronic systemic inflammation, atherosclerosis, emphysema, asthma, pulmonary fibrosis, cystic fibrosis, lupus, obstructive lung disease, pulmonary inflammatory disorder, lung cancer or other diseases having pulmonary manifestations) when they do not manifest clinically relevant signs, symptoms and/or measures of those disorders. In another embodiment, an individual or a population of individuals may be considered as not having lung disease or impaired lung function, such as COPD, when they have a FEV₁/FVC ratio (also known as FEV1/FVC ratio or FEV/FVC ratio) greater than or equal to about 0.70 or 0.72 or 0.75. In another embodiment, an individual or population of individuals that may be considered as not having lung disease or impaired lung function are sex- and age-matched with test subjects (e.g., age matched to 5 or 10 year bands) that are current or former cigarette smokers or never-smokers without apparent lung disease who have an FEV1/FVC≧0.70 or ≧0.75. Individuals or populations of individuals without lung disease or impaired lung function may be employed to establish the normal range of sequence variations (e.g., allele patterns and allele frequencies in “control subjects”) proteins, peptides or gene expression. Individuals or populations of individuals without lung disease or impaired lung function may also provide samples against which to compare one or more samples taken from a subject (e.g., samples taken at one or more different first and second times) whose lung disease or lung function status may be unknown. In other embodiments, an individual or a population of individuals may be considered as having lung disease or impaired lung function when they do not meet the criteria of one or more of the above mentioned embodiments.

In one embodiment, control subjects, as that term is used herein are sex- and age-matched current or former cigarette smokers or never-smokers, without apparent lung disease who have FEV1/FVC≧0.70. Age matching may be conducted in bands of several years, including 5, 10 or 15 year bands. Control subjects are preferably recruited from the same clinical settings. A control group is more than one, and preferably a statistically significant number of control subjects. In one embodiment, control subjects are sex- and age-matched (in 10 year bands) current or former cigarette smokers, without apparent lung disease who had FEV1/FVC≧0.70.

In one embodiment, a control sample is a sample from one or more control subjects or which provides a result representative of tests conducted on a control group. In another embodiment, a control sample is a sample from a subject without lung disease (e.g., COPD) or which provides a result representative of tests conducted on a subjects without lung disease. In another embodiment a control sample is a sample containing a known amount (e.g., in mass, number of moles, or concentration) of one or more nucleic acids and/or proteins.

In an embodiment the methods of detecting a predisposition to, a diagnosis of, a prognosis of, the response to treatment for a pulmonary disease, or predicting/determining the severity of a pulmonary disease (e.g., COPD) employ at least one, two, three, four, five, six, seven, eight, nine, ten, fifteen, or twenty sequence variations found in the nineteen chromosomal regions. In another embodiment, the methods of detecting a predisposition to, diagnosis of, or prognosis of lung disease, such as COPD, employ at least one, two, three, four, five, ten, fifteen, twenty, twenty five, or thirty of the SNPs in Tables 5a, 5b, 7, 8 and/or in FIG. 8. In another embodiment, such methods are based on detecting the presence of sequence variations in one or more, two or more, three or more, four or more, five or more, or six or more regions selected from the regions encoding CLEC4A, CSMD1, DNAH3, EBF2, ELMO1, ENPP6, KBTBD9, MSRB3, MYO5B, ENPP6 and TSC2. In another embodiment, such methods are based on detecting the presence of sequence variations in one or more, two or more, three or more, four or more, five or more, or six or more regions selected from the regions encoding CSMD1, MYO5B, DNAH3 CLEC4A, EBF2, ELMO1, and TSC2 genes. In another embodiment, such methods employ one or more, two or more, or three or more regions selected from the regions encoding: ENPP6, CSMD1, MYO5B, and DNAH3; or one or more, two or more, or three or more regions selected from the regions encoding CLEC4A, EBF2, ELMO1, and TSC2.

Assessing a number of different variations present in the nineteen chromosomal regions (e.g., the alleles from a collection of single polymorphisms) allows increased statistical confidence that the variations (e.g., SNPs) observed are indicative of the likelihood that an individual will develop pulmonary disease (e.g., COPD), can be diagnosed with pulmonary disease, or can be provided with a prognosis of the future severity of pulmonary disease. In other words, employing multiple variations in the analysis of a single subject provides increased reliability in the risk profiling of that subject. More broadly, this is analogous to the situation of an individual having only one risk factor predisposing to atherosclerosis (elevated cholesterol) vs. multiple risk factors (elevated cholesterol plus hypertension, obesity, smoking, diabetes, etc.). Risk is increased as the number of risk factors increases. Moreover, where an individual is already experiencing clinical manifestations (symptoms) of pulmonary disease, and particularly COPD, by assaying variations in nucleotide sequences in the nineteen chromosomal regions (e.g., the polymorphisms provided herein) it is possible to provide a prognosis based upon the predicted risk of developing pulmonary disease (e.g., COPD).

By assaying the polymorphisms as provided herein, it is possible to predict the risk of developing pulmonary disease (e.g., COPD) prior to its clinical detection. Such early prediction provides the clinician with opportunities to prevent the manifestation of, slow, or halt the progression of the disease.

The skilled artisan will recognize that, due to the heterogeneous nature of pulmonary diseases such as COPD, not all individuals with pulmonary disease will possess alleles for any or all of the sequence variations described herein, (e.g., SNPs listed in Tables 5a, 5b, 7 and/or 8). In some embodiments of the methods provided herein, the presence of at least three alleles, selected from the SNPs and genes shown in Tables 5a, 5b, 7, 8 and/or in FIG. 8 are assayed. The aggregate state of the variations observed (e.g., polymorphisms in SNPs) in a subject sample can provide an estimate of risk of developing a lung disease such as COPD, which may be triggered by an insult such as exposure to inhaled substances. The greater the number of biologically significant variations (e.g., polymorphisms) that are present, the greater a subject's risk of developing pulmonary disease, having pulmonary disease, or developing severe pulmonary disease (e.g., having severe symptoms of pulmonary disease such as COPD). As more polymorphisms listed in Tables 5a, 5b, 7, 8 and/or in FIG. 8 are measured, even more accurate risk profiling is possible. Thus, in other embodiments of the methods provided herein, at least about four, five, six, seven, eight, nine, ten, fifteen, twenty or twenty-five variations such as SNPs are examined in determining a predisposition to, providing a prognosis or diagnosis of, or predicting/determining the severity of pulmonary diseases such as COPD.

Where it is desirable, sequence variations within the nineteen chromosomal regions identified, and all other sources of variation in associated regions, may be used to calculate a measure quantifying the risk of developing a disease (COPD), diagnosing it, or predicting its progression or severity. This calculation is conducted by an algorithm where the individual variations identified in a subject are used alone or in combination in the calculation. The result would quantify risk as an Odds Ratio (OR) or a Predictive Probability (PP). Further, the calculation of such a combined outcome could include other non-genetic variables including, but limited to, demographics, exposure, and biomarkers such as age, ancestry, cumulative exposure to cigarette smoke, spirometric measures of lung function, presence of symptoms such as, but not limited to, dyspnea, measure of exercise capacity, gene expression level, protein abundance, metabolite levels, or methylation status. A combination of multiple variables, including those yet to be identified will increase the accuracy of the assessment.

### 4.0 Prevention and Treatment of Pulmonary Diseases

The linkage (association) of variations in different portions of the nineteen chromosomal regions (e.g., genes) described herein with the development of pulmonary diseases such as COPD and their progress, indicates that different polymorphisms may play a role in the development of pulmonary diseases in different subjects. As variations at different polymorphic sites will occur in different subjects, the associations between various genetic sites provided herein make possible the identification of subject profiles (e.g., profiling of patients). Such subject profiles make possible individualized treatments, which are desirable as regimes effective to treat a first patient with a first profile may not be as effective in a second patient with a different second profile. Subject specific profiles also allow less effective (or ineffective) treatments, particularly those accompanied by undesirable side effects, to be avoided.

In view of the correlation between the etiology of COPD and genes associated with identified sequence variations (e.g., SNPs) within identified chromosomal regions, the ability to manipulate the expression of those genes represents an efficacious means to treat pulmonary disease such as COPD. Methods to treat a pulmonary disease may include gene therapy to increase or decrease the expression of the level or activity of one or more of the gene products produced by the genes found in chromosomal regions identified herein. Treatment may also include methods in addition to, or as an alternative to, gene therapy to increase or decrease the expression or activity of one or more products of the genes found in the chromosomal regions identified herein.

The products of genes in the nineteen chromosomal regions identified herein are not limited to nucleic acids. Identification of genes involved in the development of pulmonary diseases such as COPD also makes possible an identification of proteins that may affect the development of a pulmonary disease. Identification of such proteins makes possible the use of methods to affect their expression, processing, abundance, function, biological activity, or to alter their metabolism. Methods to alter the effect of expressed proteins include, but are not limited to, the use of specific antibodies or antibody fragments that bind the identified proteins, specific receptors that bind the identified proteins, or other ligands or small molecules that inhibit the identified proteins from affecting their physiological target and exerting their metabolic and biologic effects. In addition, those proteins that are down-regulated or are affected by mutations reducing their activity may be exogenously supplemented to ameliorate the effects of their decreased activity or synthesis, or increased degradation. The identification of genes involved in the development of pulmonary diseases also makes possible prophylactic methods to affect gene expression or protein function that may be used to treat individuals at risk for the development of a pulmonary disease, or to prevent the clinical manifestation of a pulmonary disease in individuals at risk for its development.

4.1 Methods of Enhancing Gene Expression

Where a subject has decreased activity of one or more gene products relative to the levels found in individuals expressing the wild type gene, it is possible to treat pulmonary diseases such as COPD by enhancing expression of one or more of those genes. Gene transcription may be deliberately modified in a number of ways to enhance the activity of the gene products in a subject. In one embodiment, exogenous copies of a gene are inserted into the genome of cells (e.g., a subject's cells) via homologous recombination in vivo or in vitro. In other embodiments, gene products may be expressed in cells by the introduction of a vector that remains extrachromosomal (e.g., a plasmid or a viral vector such as modified adenovirus), thereby allowing for transcription and expression independent of the genomic allele. Yet another method is transfection with naked DNA. In some embodiments, a promoter specific to the vector, rather than a copy of the wild type promoter, is used to drive expression of the gene product from the vector.

Where the genes are inserted into cells in vitro, the resulting cells can be introduced into a subject. Transient expression from introduced vectors generally have high expression levels; however, the gene/vector is maintained for a short period of time, particularly without selection, although use of an episomal vector containing a eukaryotic origin of transcription provides for greater persistence of the vector.

4.2 Methods of Inhibiting Gene Expression

Where a subject has increased activity of one or more gene products relative to the levels found in individuals expressing the wild type gene, it is possible to treat pulmonary diseases such as COPD by inhibiting expression of those genes or increasing the degradation of the gene products. Treatments to decrease gene expression, particularly by increasing the degradation of the gene products, include, but are not limited to, the expression of anti-sense mRNA, triplex formation, inhibition by co-expression, and administration or expression of siRNA. Thus, in one embodiment, antisense RNA introduced into a cell binds to complementary mRNA and inhibits the translation of that molecule. In another embodiment, antisense single stranded cDNA introduced into a cell inhibits the translation, and possibly speeds degradation of the DNA-RNA duplex. In another embodiment, short interfering RNAs (RNAi or siRNA) specifically inhibit gene expression. See Tuschl et al., Nature 411:494-498 (2001). In another embodiment, stable triple-helical structures can be formed by bonding of oligodeoxyribonucleotides (ODNs) to polypurine tracts of double stranded DNA. See, for example, Rininsland, Proc. Nat'l Acad. Sci. USA 94:5854-5859 (1997). Triplex formation can inhibit DNA replication by inhibition of transcription of elongation and is a very stable molecule.

4.3 Methods to Enhance the Activity of Specific Proteins

Where it is desirable to enhance the activity of proteins in a subject the proteins themselves may be administered to the subject. Alternatively, the subject may be treated, as described above, to introduce one or more copies of nucleic acids encoding the protein. Where the protein encodes an enzyme, it is even possible to supply the product of the transformation catalyzed by the enzyme.

4.4 Methods to Inhibit the Activity of Specific Proteins

In those instances where it is desirable to reduce the level or activity of one or more proteins produced by the genes in the chromosomal regions described herein to treat pulmonary diseases, the proteins can be reduced with an agent having affinity for the protein. Such agents include, but are not limited to, monoclonal antibodies, polyclonal antibodies, multispecific antibodies (e.g., bispecific antibodies) or a fragment thereof, including but not limited to an scFv, a Fab fragment, a Fab′ fragment, a F(ab′)₂, an Fv, and a disulfide linked Fv.

In one embodiment, specific antibodies, or fragments thereof, may be used to bind the protein thereby blocking its activity. Such antibodies may be obtained through the use of conventional techniques, including hybridoma technology, or may be isolated from libraries commercially available (e.g., libraries from Dynax (Cambridge, Mass.), MorphoSys (Martinsried, Germany), Biosite (San Diego, Calif.) and Cambridge Antibody Technology (Cambridge, UK)). In addition, where the protein in question interacts with another protein, such as a cellular receptor, antibodies that antagonize the interaction between the specific protein and the cellular receptor can be used to block interactions that lead to the development of COPD and other pulmonary diseases.

### 5.0 Compositions and Kits

5.1 Nucleic Acids

The present disclosure encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in Tables 5a, 5b, 7, 8 and/or in FIG. 8. Furthermore, kits/systems (such as beads, arrays, etc.) that include these analogs are also encompassed. For example, PNA oligomers that are based on the polymorphic sequences of the present disclosure are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994); Petersen et al., Bioorganic & Medicinal Chemistry Letters, 6: 793-796 (1996); Kumar et al., Organic Letters 3(9): 1269-1272 (2001); WO96/04000). PNAs hybridize to complementary RNA or DNA with higher affinity and specificity than conventional oligonucleotides and oligonucleotide analogs.

Additional examples of nucleic acid modifications that improve the binding properties and/or stability of a nucleic acid include use of base analogs such as inosine, intercalators (U.S. Pat. No. 4,835,263) and minor groove binders (U.S. Pat. No. 5,801,115). Thus, references herein to nucleic acid molecules, SNP-containing nucleic acid molecules, SNP detection reagents (e.g., probes and primers), and oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

The term “target nucleic acid” can include any nucleic acid sequence to be detected in an assay. The “target nucleic acid” may comprise the entire sequence of interest (e.g., one or more of the nineteen chromosomal regions identified herein) or may be a sub-sequence (e.g., a fragment) of the nucleic acid target molecule, such as a nucleotide sequence wherein a variation such as a SNP may be present. In an embodiment, the portion of a target nucleic acid may be in a range selected from: 25 to 50 base pairs, 30 to 60 base pairs, 40 to 80 base pairs, 40 to 100 base pairs, 50 to 200 base pairs, 60 to 300 base pairs. 70 to 500 base pairs, 80 to 800 base pairs, 100 to 1,000 base pairs, 200 to 4,000 base pairs, 500 to 10,000 base pairs, and 1,000 to 20,000 base pairs of chromosomal regions 1-19 (see, e.g., FIG. 8).

5.1 Nucleotide Probes and Primers

The present disclosure includes and provides for nucleic acid molecules that may be used to detect variations in the nucleotide sequences of the nineteen regions identified herein, including both probes and primers.

Nucleic acid probes include any oligomer of RNA, DNA, or PNA, suitable for hybridizing to all or a portion of the target nucleic acid (DNA or RNA) that can be used to initiate the synthesis of a nucleic acid molecule that is complementary to the sequence of that target. Alternatively, nucleic acid probes include any oligomer of RNA, DNA, or PNA that can be used to detect variations in the sequence of the target nucleic acid. In some embodiments, nucleic acid probes can be, for example, a primer suitable for use in methods where a DNA polymerase extends the primer, such as in polymerase chain reaction (PCR) or variants thereof (e.g., hot start PCR). Such primers may be labeled with a detectable moiety or may be unlabeled. Likewise, a primer may be in solution or immobilized to a solid support or solid carrier. In some embodiments, a suitable primer can also be a suitable probe. In some embodiments, a suitable probe can be a suitable primer.

Nucleic acids of the present disclosure include and provide for nucleic acids in the form of a composition, such as a kit, comprising two or more nucleic acid probes for the identification of one or more variations in a nucleotide sequence of one or more chromosomal regions selected independently from regions 1-19. Such kits optionally comprise instructions for the use of the kit to identify one or more of said variations and/or one or more control nucleic acids for said variations in said nucleotide sequence. In one embodiment, the control is a nucleic acid. In another embodiment, the control is selected from the group consisting of homozygous reference genotype, homozygous variant genotype, heterozygous genotype, and combinations thereof for the SNPs identified by the probes. In another embodiment, one or more nucleic acids in a kit or composition bind to a region adjacent to a SNP or variation (e.g., within a distance that the nucleic acid can be used as a nucleic acid primer for detecting or amplifying the SNP or variation, or within 1, 10, 20, 30, 50, 100, 200, 300, 400 or 500 base pairs of the SNP or variation) present in chromosomal regions 1-19. In yet another embodiment of a kit or composition, at least one, two, three, four, five, or six different nucleotide is suitable for use as primers for the amplification of a nucleic acid sequences within one or more of chromosome regions 1-19 (e.g., the nucleic acids are different PCR or LCR primers). In such an embodiment, the nucleic acids comprise a nucleotide sequence that is complementary to at least one strand of the nucleotide sequence of said chromosomal regions.

The nucleic acid molecules of the kits can include a probe that is capable of detecting all or a portion of a given target nucleic acid sequence, such as a SNP sequence. The nucleic acid molecule can include a nucleic acid sequence that is longer than a given SNP sequence. In some embodiments, the kits include instructions for preparing the samples for analysis using the kit. In some embodiments, the kits include instructions for analyzing and/or interpreting the results obtained using the kit.

Nucleic acid probes may be any suitable nucleic acid (polynucleotide) molecule. Suitable nucleic acid probes include any oligomer, comprising two or more nucleobases containing subunits, such as a polynucleotide (RNA or DNA) or synthetic polynucleotide mimetics such as peptide nucleic acids (PNA). In some embodiments nucleic acid probes may contain greater than about 10, 12, 14, 15, 16, 17, 18, 20, 22, or 24 nucleobases containing subunits and less than about 26, 28, 30, 32, 34, 36, 40, 44, 48 or 50 nucleobases. In other embodiments, the probes may contain greater than about 18, 20, 22, 24, 26, or 28 nucleotides and less than about 100, 200 300, 400 or 500, 750 or 1,000 nucleobases containing subunits. Nucleic acid probes, whether comprising DNA, RNA or synthetic mimetics can hybridize to all or a portion of the target nucleic acid (DNA or RNA). Probes may be labeled with a detectable moiety (e.g., fluorescent tags or isotope labels) or may be unlabeled. Likewise, a probe may be in solution or immobilized to a solid support or solid carrier. In one embodiment, compositions comprising probes may comprise nucleic acid sequences from two, three, four, five, six, seven, eight or more different chromosomal regions of the nineteen chromosomal regions identified herein (see e.g., FIG. 8). In another embodiment, the compositions may comprise four, five, six, seven, eight or more probes, wherein said probes comprise at least two primers from a first region selected from the 19 regions set forth in FIG. 8, and two primers from a second region selected from the nineteen regions set forth in FIG. 8, where the first and second regions are different.

The present disclosure also provides compositions comprising two or more pairs of nucleic acid molecules that may be, for instance, pairs of primers for amplification of various portions of chromosomal regions 1-19. In such embodiments, the two or more pairs of nucleic acid molecules comprise a first pair of nucleic acid molecules and a second pair of nucleic acid molecules. The first pair of nucleic acid molecules comprises a first nucleic acid molecule comprising a nucleotide sequence complementary to a portion of a chromosomal region selected from chromosomal regions 1-19 and a second nucleic acid molecule comprising a nucleotide sequence complementary to the opposite strand of the chromosomal region to which said first nucleic acid is complementary. The second pair of nucleic acid molecules comprises a third nucleic acid molecule comprising a nucleotide sequence complementary to a portion of a chromosomal region selected from chromosomal regions 1-19 and a fourth nucleic acid molecule comprising a nucleotide sequence complementary to the opposite strand of the chromosomal region to which said third nucleic acid is complementary. Such compositions may contain additional pairs of nucleic acid molecules.

5.2 Pharmaceutical Compositions Comprising Nucleic Acids

The linkage of specific chromosomal regions, including specific genes, to pulmonary diseases provides a basis for new therapeutic compositions. Those compositions may be directed, for example, at the genes or their products, and may be used to inhibit, slow, or prevent lung diseases such as COPD. For instance, the pharmaceutical compositions may comprise one or more of a gene product of CLEC4A, CSMD1, DNAH3, EBF2, ELMO1, ENPP6, KBTBD9, MSRB3, MYO5B, or TSC2. Such compositions may be useful to treat subjects suffering from pulmonary diseases such as COPD and may even be used prophylactically to treat individuals with a predisposition to the development of COPD (e.g., to prevent the development of COPD triggered by exposure to inhalation of noxious substances).

5.3. Antibodies and Composition Comprising Antibodies

The term antibody includes any naturally occurring (e.g., monospecific polyclonal) or man-made antibodies such as monoclonal antibodies produced by conventional hybridoma technology. The term antibody also includes fragments or portions of antibodies that contain the antigen-binding domain and/or one or more complementarity determining regions of these antibodies, including but not limited to a scFv, a Fab fragment, a Fab′ fragment, a F(ab′)₂, an Fv, or a disulfide linked Fv. The term antibody refers to any form of antibody, or fragment thereof, that specifically binds to an antigen such as an antigen of the gene product of any one of KBTBD9, MSRB3, TSC2, CLEC4A, CSMD1, DNAH3, EBF2, ELMO1, MYO5B, and ENPP6, and specifically covers monoclonal antibodies (including full length monoclonal antibodies), polyclonal antibodies, multispecific antibodies (e.g., bispecific antibodies), Fab(s), Fab′(s), single chain antibodies, diabodies, domain antibodies, miniantibodies, or an antigen binding fragment of any of the foregoing. Any specific antibody or fragment thereof can be used in the methods and compositions provided herein including but not limited to an scFv, a Fab fragment, a Fab′ fragment, a F(ab′)₂, an Fv, a disulfide linked Fv, an Fab(s), an Fab′(s), a single chain antibodies, diabodies, domain antibodies, miniantibodies, or antigen binding fragments of any of the foregoing. Thus, in one embodiment the term “antibody” encompasses a molecule comprising at least one variable region from a light chain immunoglobulin molecule and at least one variable region from a heavy chain molecule that in combination form a specific binding site for the target antigen. In some embodiments, antibodies may also be an IgA, IgD, IgE, IgG or IgM or any combination thereof, including combinations of subtypes of those antibodies. In one embodiment, the antibody is an IgG antibody; for example, the antibody can be an IgG1, IgG2, IgG3, or IgG4 antibody.

The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, or apes. See generally, Harlow, E. & Lane, E. (1988) Antibodies: A Laboratory Manual (Cold Spring Harbor Press, Cold Spring Harbor, N.Y.). In one embodiment, an antibody is a mammalian antibody. In another embodiment, phage display techniques can be used to screen for and isolate an initial antibody or to generate variants with altered specificity or avidity characteristics. Such techniques are routine and well known in the art. See e.g., U.S. Pat. No. 6,172,197.

In other embodiments, antibodies are produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., MONOCLONAL ANTIBODIES (Oxford University Press, 2000); Goding, Monoclonal Antibodies: Principles And Practice (Academic Press, 1993); Current Protocols In Immunology (John Wiley & Sons, most recent edition). A suitable antibody can also be modified by recombinant means to increase greater efficacy of the antibody in mediating the desired function. Antibody fragments or portions thereof include at least a portion of the variable region of the immunoglobulin molecule that binds to its target, i.e., the antigen binding region. An antibody can be in the form of an antigen binding antibody fragment including a Fab fragment, F(ab′)2 fragment, a single chain variable region, and the like. Fragments of intact molecules can be generated using methods well known in the art including enzymatic digestion and recombinant means.

The antibodies or antigen binding fragments thereof provided herein may be conjugated to a “bioactive agent.” As used herein, the term “bioactive agent” refers to any synthetic or naturally occurring compound that binds the antigen and/or enhances or mediates a desired biological effect to enhance cell-killing toxins, or can be an agent used to detect the antibody in vitro or in vivo. Bioactive agents include, but are not limited to, enzymes (e.g., ricin or portions and modified forms thereof), radiolabels, and sensitizers such as agents useful for photodynamic therapy such as aminolevulinic acid (ALA), phthalocyanines, (e.g., silicon phthalocyanine Pc 4), and m-tetrahydroxyphenylchlorin.

The compositions, methods, kits and the like, thus generally described, will be further understood by reference to the following examples, which are provided by way of illustration and are not intended to be limiting.

### 6.0 Example 1

To identify genetic risk factors for COPD, a GWAS was performed in a sample of 192 adult smokers with COPD by spirometry and in 197 control subjects (90 smokers and 107 never smokers). Outcomes analyzed were 4 spirometry-based indices that deconvolute the major pathophysiologic factors associated with COPD, including baseline lung function (BL), age-related decline (Age decline), pack-years-related decline (Pack-years decline), and the intensifying effects of smoking, in terms of number of cigarettes per day (CPD), on decline with age decline (Pack-years decline). The minimum p-values were 8.5×10⁻⁶ (BL), 2.33×10⁻⁷ (Age decline), 1.90×10⁻⁶ (Pack-years decline), 1.90×10⁻⁶ (CPD×Age decline). False discovery rate (FDR) analysis showed that Age decline and Pack-years decline were enriched for significant associations. A minimum SNP-specific FDR (q-value) of 0.124 was found within the gene ENPP6 for Age decline. A total of 33 SNPs had q-values less than 0.5, with most being associated with Pack-years decline. As shown in FIG. 8, clusters of associated SNPs were found in several genes.

6.1 Methods

6.1.1 Study Sample

Cases were obtained from a subset of the Lung Health Study (LHS), a prospective, randomized, multicenter, clinical trial in the US and Canada conducted in two phases between 1986 and 2001 (LHS-1 and LHS-3) (Buist et al. 1993, Chest 103 (6):1863-1872; Anthonisen et al. 1994, JAMA 272:1497-1505; Anthonisen et al. 2002, Am. J. Respir. Crit. Care Med. 166:675-679). Participants in LHS-1 were otherwise healthy cigarette smokers, aged 35 to 60 years, with mild or moderate COPD as determined by spirometry (ratio of forced expiratory volume in 1 second (FEV₁) to forced vital capacity (FVC)<0.70 and FEV₁ 55% to 90% of predicted) (National Institutes of Health and National Heart Lung and Blood Institute 2007). At the University of Utah center, 624 participants enrolled in LHS-1, and 503 completed LHS-3. Of these, 192 had genotyping performed in a follow-on, cross-sectional, genetic association study, the Genetics of Addiction Project (GAP), during 2003-2005. GAP also included 197 gender- and age-matched controls (90 smoked cigarettes and 107 never smoked).

6.1.2 Lung Function Decline Outcome Measures

Four quantitative spirometry-based indices of lung function decline in the study sample, best linear unbiased predictors (BLUPS), were derived from longitudinal mixed growth curve modeling as a function of major COPD risk factors and is described herein. (The general statistical approach is described in Robinson 1991; Goldstein H. Multilevel statistical models. New York: Wiley, 1995.) Mixed models specifically designed for the analysis of clustered data and that estimate two types of parameters, fixed and random effects were used (Demidenko 2004, Mixed models: theory and applications. Wiley: Hoboken, N.J.). Fixed effects are analogous to regression coefficients, while random effects describe the degree to which an individual subject's coefficient value deviates from the fixed effect.

6.1.3 Data Analysis and Modeling

Data were modeled for 624 cigarette smokers with COPD and aged 35-60 at baseline, followed up 7 times over approximately 17 years (1986-2004) in the Lung Health Studies (Anthonisen et al., 1994; Connett et al., 1993, Control. Clin. Trials 14:3S-19S) and its follow-on Genetics of Addiction Project (GAP); 204 GAP subjects without COPD were also studied as controls (see Table 1 for descriptive statistics). The optimal model of the data was selected based on likelihood ratio tests, which were used to determine the significance of each fixed and random effect parameter as it was added to the model (Willet et al., 1998). After the optimal model was identified, the outcome variables were calculated as best linear unbiased predictors (BLUPs) of the random effects. Missing data were handled by multiple imputation using chained equations, with 5 datasets imputed and analyzed (Van Buuren et al. 2006, Journal of Statistical Computation and Simulation 2006; 76(12): 1049-1064; Royston 2005, Stata Journal 5(4): 527-536).

TABLE 1
Descriptive statistics of subject characteristics at study initiation*

| | Female (N = 303) | | Male (N = 525) | |
|---|---|---|---|---|
| Variables | Mean ± SD | Range | Mean ± SD | Range |
| Age (y) | 44.82 ± 8.08 | 26-60 | 46.59 ± 7.47 | 28-68 |
| FEV₁ (L) | 2.44 ± 0.52 | 1.18-3.93 | 3.16 ± 0.63 | 1.02-6.09 |
| Height (cm) | 164.01 ± 5.88 | 150-180 | 176.89 ± 6.37 | 151-197 |
| Pack-years | 28.41 ± 20.44 | 0-87.5 | 38.14 ± 23.29 | 0-153 |
| CPD | 0.58 ± 0.60 | 0-2.71 | 0.77 ± 0.67 | 0-4 |
| Never smoked | 0.21 | 0-1 | 0.09 | 0-1 |

| | | |
|---|---|---|
| Total missing data, all | 8.81% | 8.73% |
| variables and waves | | |

CPD, cigarettes per day.
Note:
Due to extremely small coefficient sizes, CPD was specified as CPD/20, thus making the measurement equivalent to packs per day; FEV₁, forced expiratory volume in 1 second; SD, standard deviation.
*Descriptive statistics calculated from non-imputed data at participant's first assessment.

In developing the random effect-based outcome measures, linear mixed models predicting forced expiratory volume in 1 second (FEV₁) were systematically developed. Linear mixed models are a generalization of linear regression allowing for the inclusion of random deviations (i.e. random effects) other than those associated with the overall residual term. In matrix notation,

y=Xβ+Zu+ε

where y is the n×1 vector of responses, X is a n×p design/covariate matrix for the fixed effect P, and Z is the n×q design/covariate matrix for the random effects u. The n×1 vector of residuals c, is assumed to be multivariate normal with mean zero and variance matrix σₑ²Iₙ.

The fixed portion, Xβ, is equivalent to the linear predictor of OLS regression. For the random portion, Zu+ε, it is assumed that the u has variance-covariance matrix G and that u is orthogonal to ε so that

$$Var [ \begin{matrix}u \\ ɛ\end{matrix} ] = [ \begin{matrix}G & 0 \\ 0 & {σ}_{e}^{2} {I}_{n}\end{matrix} ]$$

The random effects u are not directly estimated (although, as described below, they may be predicted), but instead are characterized by the elements of G, known as the variance components, that are estimated along with the residual variance σₑ². Considering Zu+c the combined error, we see that y is multivariate normal with mean Xβ and n×n variance-covariance matrix

V=ZGZ′+σₑ²Iₙ

The model building process is shown in Table 2. The outcome measures used in this analysis were derived from the random effects of the final, best-fitting model:

yᵢⱼ=β₀+β₁x₁ᵢⱼ+β₂x₂ᵢⱼ+β₃x₃ᵢⱼ+β₄x₄ᵢⱼ+β₅x₅ᵢⱼ+β₆x₆ᵢⱼ+β₇x₇ᵢⱼ+u₀ᵢ+u₁ᵢ+u₂ᵢ+u₃ᵢ+eᵢⱼ

where i indexes subjects, j indexes repeated assessments, y is FEV₁, β₀ is the intercept fixed effect, x₁ is age, β₁ is the age fixed effect, x₂ is pack years, β₂ is the pack years fixed effect, x₃ is CPD×age, β₃ is the cpd×age fixed effect, x₄ is height, β₄ is the height fixed effect, x₅ is gender, β₅ is the gender fixed effect, x₆ is gender×age, β₆ is the gender×age fixed effect, x₇ is never-smoked status, β₇ is the never-smoked status fixed effect, u₀ᵢ is the intercept random effect, u₁ᵢ is the age random effect, u₂ᵢ is the pack years random effect, u₃ᵢ is the CPD×age random effect and eᵢⱼ is the within-subject residual. Parameter estimates and p-values for the final model (shown in Table 2 as Model 15) are shown in Table 3.

TABLE 2
Results of FEV₁ linear mixed modeling

| | | Test | | vs. | |
|---|---|---|---|---|---|
| Model | Variables | statistic* | df^{†} | Model | p-value |
| 1 | Intercept | — | — | — | — |
| 2 | Model 1 + Random Intercept | 2423.13 | 1, 41 | 1 | <.001 |
| 3 | Model 2 + Age | 992.28 | 1, 25 | 2 | <.001 |
| 4 | Model 3 + Random Age | 99.30 | 1, 159 | 3 | <.001 |
| 5 | Model 4 + Unstructured RE covariance | 122.74 | 1, 128 | 4 | <.001 |
| 6 | Model 4 + Age² | 2.48 | 1, 17 | 5 | NS |
| 7 | Model 5 + Height | 283.98 | 1, 110 | 5 | <.001 |
| 8 | Model 6 + Male | 26.38 | 1, 137 | 7 | <.001 |
| 9 | Model 7 + Male × Age | 15.00 | 1, 1144 | 8 | <.001 |
| 10 | Model 8 + Height × Age | 3.80 | 1, 65 | 9 | NS |
| 11 | Model 8 + Pack-years | 14.56 | 1, 6 | 9 | <.01 |
| 12 | Model 10 + Random Pack-years | 51.35 | 1, 7 | 11 | <.001 |
| 13 | Model 11 + CPD × Age | 7.89 | 1, 7 | 12 | <.05 |
| 14 | Model 11 + Random CPD × Age | 27.96 | 1, 18 | 13 | <.001 |
| 15 | Model 12 + Never smoked | 104.69 | 1, 248 | 14 | <.001 |
| 16 | Model 13 + CPD | 1.03 | 1, 41 | 15 | NS |
| 17 | Model 13 + Pack-years × Age | 0.46 | 1, 164 | 15 | NS |
| 18 | Model 13 + Never smoked × Age | 0.36 | 1, 19779 | 15 | NS |

CPD, cigarettes per day.
Note:
Due to extremely small coefficient sizes, CPD was specified as CPD/20, thus making the measurement equivalent to packs per day; FEV₁, forced expiratory volume in 1 second; RE, random effect; NS, not significant.
*This is the multiple imputation version of the likelihood ratio test statistic (Allison, P. Thousand Oaks, CA: Sage Publications, 2001). The test statistic approximates an F-distribution under the null hypothesis. See Bollen and Curran (Latent curve models: A structural equation approach. Hoboken, NJ: Wiley, 2006) for test statistic and degrees of freedom equations.
^{†}Two values are given for the degrees of freedom as the test statistic has an F-distribution.

The covariance structure of the four random effects was modeled as unstructured:

$$[ \begin{matrix}{u}_{0 i} \\ {u}_{1 i} \\ {u}_{2 i} \\ {u}_{3 i}\end{matrix} ] \text{∼} N ( 0 , G ) with G = [ \begin{matrix}{σ}_{u 0}^{2} & & & \\ {σ}_{u 10} & {σ}_{u 1}^{2} & & \\ {σ}_{u 20} & {σ}_{u 21} & {σ}_{u 2}^{2} & \\ {σ}_{u 30} & {σ}_{u 31} & {σ}_{u 32} & {σ}_{u 3}^{2}\end{matrix} ]$$

Thus, the random parameters are multivariate normal distributed with means of zero and variance-covariance matrix G. The variances of the parameters are on the diagonal and the covariances in the off-diagonal cells of G. The residual is assumed to be normally distributed with a mean of zero and variance of σ²ₑ.

Because random effects are not directly estimated by the mixed model, they must be predicted in an additional post-estimation step. BLUPs of the random effects u were obtained as

ũ=\tilde{G}Z′\tilde{V}⁻¹(y−X\hat{β})

where \tilde{G} and \tilde{V} are G and V with estimates of the variance components plugged in. The EM algorithm was used for maximum likelihood estimation as described by Pinheiro and Bates (Mixed-Effects Models in S and S-PLUS. Berlin: Springer, 2000).

TABLE 3
Parameter estimates and statistical significance
of final linear mixed model of FEV₁

| | | |
|---|---|---|
| Parameters | SE | p-value |

Fixed Effects

| | | | |
|---|---|---|---|
| Intercept (L) | 2.960 | 0.047 | <.001 |
| Age (y) | −0.027 | 0.002 | <.001 |
| Height (cm) | 0.031 | 0.002 | <.001 |
| Male Gender | 0.542 | 0.055 | <.001 |
| Height × Age | −0.009 | 0.002 | <.001 |
| Pack-years | −0.002 | 0.001 | <.05 |
| CPD × Age | −0.003 | 0.000 | <.01 |
| Never smoked | 0.780 | 0.064 | <.001 |

Random Effects

| | | | |
|---|---|---|---|
| SD (Intercept) | 0.505 | 0.031 | <.001 |
| SD (Age) | 0.021 | 0.001 | <.001 |
| SD (Pack-years) | 0.008 | 0.002 | <.001 |
| SD (CPD × Age) | 0.007 | 0.001 | <.001 |

CPD, cigarettes per day.
Note:
Due to extremely small coefficient sizes, CPD was specified as CPD/20, thus making the measurement equivalent to packs per day; FEV₁, forced expiratory volume in 1 second; SD, standard deviation; SE, standard error.

The best-fitting model showed significant random effects for baseline lung function, age, pack-years (product of the average number of packs smoked daily and the total years of smoking), and the interaction between age and recent smoking as estimated by the number of cigarettes smoked daily. The effect size for each of these factors varied considerably across subjects. BLUPs for baseline lung function (BL), age-related decline (Age decline), Pack-years-related decline (Pack-years decline), and the interaction between age and smoke-related decline (CPD×Age decline) were calculated for these four significant random effects and served as the outcome measures in the GWAS. The mean correlation among the BLUPs was −0.22, suggesting that they reflected independent biological effects. These more homogenous, independent measures are useful compared to composite measures that can confound distinct mechanisms and can result in a loss of statistical power.

6.1.4 Sample Collection and Preparation and Genotyping

A whole blood sample was collected by venipuncture from each subject in an EDTA vacutainer tube. DNA was extracted from white blood cells, purified (Puregene Kit, Gentra Systems, Inc, Minneapolis, Minn.), and stored at −70° C. Genotyping was performed in accordance with manufacturer-recommended procedures using the Infinium II HumanHap 550 SNP array (Illumina, San Diego, Calif.) on a BeadStation. Robotic liquid handling stations were used for sample handling. The HumanHap 550 array assays 555,352 tagging SNPs selected from Phases I and II of the HapMap Project. Genotypes were called using BeadStudio genotyping module version 3.2.32. The mean call rate of arrays in the analysis was 0.998, and arrays with a fail rate above 0.980 were repeated.

6.1.5 Association Analysis

All association analyses were performed in PLINK. The minimum allowable SNP and individual genotyping success rates were 0.95. The minimum allowable observed SNP minor allele frequency (MAF) was 0.025.

To control the risk of false discovery, for each significant BLUP-based SNP association a q-value was calculated. A q-value is an estimate of the proportion of false discoveries, or FDR, among all significant markers when the corresponding p-value is used as the threshold for declaring significance (Storey 2003, Ann. Stat. (31):2013-2035; Storey and Tibshirani 2003, Proc. Natl. Acad. Sci. U.S.A. 100 (16):9440-9445). This FDR-based approach (1) provides a good balance between the competing goals of true positive findings versus false discoveries, (2) allows the use of more similar standards in terms of the proportion of false discoveries produced across studies because it is much less dependent on the arbitrary number, or sets, or statistical tests that are performed, (3) is relatively robust against the effects of correlated tests, and (4) provides a more subtle picture about the possible relevance of the tested markers rather than an all-or-nothing conclusion about whether a study produces significant results (Benjamini and Hochberg 1995, Journal of the Royal Statistical Society B 57:289-300; Brown and Russell 1997, Statistics in Med. 16 (22):2511-2528; Storey 2003, Ann. Stat. (31):2013-2035; Sabatti, Service, and Freimer 2003, Genetics 164 (2):829-833; Tsai, Hsueh, and Chen 2003, Biometrics. 59 (4):1071-1081; van den Oord and Sullivan 2003, Human Heredity 56 (4):188-189; Fernando et al. 2004, Genetics 166 (1):611-619; Korn et al. 2004, Journal of Statistical Planning and Inference 124 (2):379-398; van den Oord 2005, Mol. Psychiatry. 10 (3):230-231). The q-values were calculated conservatively assuming p₀=1. For each BLUP-based association an estimate of the proportion of null effects (p0) was calculated using two estimators known to perform best in GWAS studies (Meinshausen and Rice 2006, The Annals of Statistics 34 (1):373-393; Kuo et al. 2007, BMC Proceedings, 1: S143).

For comparison with the BLUP-based association results, a secondary analysis was performed using as outcomes the statistically less powerful traditional case-control categories and the FEV₁/FVC ratio by which COPD is operationally defined.

6.1.6 Stratification

All subjects were Caucasian, but there could be genetic subgroups in the sample. Population substructure could result in false positive findings if the subgroups differed in allele frequencies, prevalence of COPD, or quantitative measures of lung function decline. A variety of methods is available to detect population substructure and correct for its potential confounding effects. Sullivan et al. (Sullivan et al. 2008, Mol. Psychiatry. 13 (6):570-584) performed an extensive evaluation of multiple statistical methods to avoid false positive findings in GWAS due to such genetic subgroups. They concluded that the principal components and multi-dimensional scaling (MDS) approaches were very similar and superior to other approaches. MDS was used for practical reasons as it can be implemented in PLINK (Purcell et al. 2007, Am. J. Hum. Genet. 81 (3):559-575).

Input data for the MDS approach were the genome-wide average proportion of alleles shared identically by state (IBS) between any two individuals. Somewhat analogous to principal component analysis, the first MDS dimension of a (genetic) similarity matrix captures the maximal variance in the genetic similarity, the second dimension must be orthogonal to the first and captures the maximum amount of residual genetic similarity, and so on. A one-dimension solution was the best-fitting model to account for the genetic similarity among subjects in this sample.

### 6.2 Results

6.2.1 GWAS Results

A total of 391 assays, each with 561,466 SNPs, was performed and passed quality control. After filtering by fail rate and minimum minor allele frequency, 518,714 SNPs were analyzed for association with the four lung function decline BLUPs. FDR analysis performed on tests of Hardy-Weinberg equilibrium using the entire sample showed a FDR of 10%, corresponding to a p-value <0.0001. An additional 3,823 SNPs had deviations from Hardy-Weinberg equilibrium below a FDR of 10%.

The minimum P values for the BLUP-based SNP associations were 8.5×10⁻⁶ (BL), 2.33×10⁻⁷ (Age decline), 1.90×10⁻⁶ (Pack-years decline), and 1.90×10⁻⁶ (CPD×Age decline). After FDR analysis, Pack-years decline and Age decline showed evidence of true effects with a minimum p0 estimate of 0.9999877. As the product of (1-p₀) and the number of markers estimates the number of effects, this suggested 0 to 8 SNPs with real effects (Table 4). In contrast, the BL and CPD×Age decline SNP associations had p0 estimates of 1 or greater, suggesting moderate inflation of false discoveries since completely null data would show a p0 equal to 1.

TABLE 4
p0 estimates for the False Discovery Rate (FDR) analysis
of the Genome Wide Association Study (GWAS) results

| | | | | | Estimated number of SNPs | | |
|---|---|---|---|---|---|---|---|
| | SNPs | p0 estimate | | | with real effects | | |
| BLUP | (n) | conservative | low | linb | conservative | low | linb |
| Pack Years | 518,714 | 1 | 0.9999846 | 0.9999877 | 0 | 8 | 6.4 |
| Age | 518,714 | 1 | 1 | 0.9999985 | 0 | 0 | 0.8 |
| Base Line | 518,714 | 1.000002 | 1 | 1.000015 | −1 | 0 | −7.6 |
| Lung | | | | | | | |
| Function | | | | | | | |
| CPD × Age | 518,714 | 1 | 1 | 1.000001 | 0 | 0 | −0.3 |

After the FDR analysis, 33 SNPs had q-values less than 0.5 (see, e.g., Tables 5a and 5b and FIG. 8). Although a q-value of 0.5 means that an average of 50% of observations were false discoveries, it is unlikely that all 33 were. The most significant q-value observed across all BLUP-based associations was for SNP rs7689305 in the gene ENPP6 for the Age Decline BLUP (p-value=2.33×10-7, q-value=0.12). Of the top 33 SNPs, 21 were clustered in 7 clusters of SNPs with LD between regions with a maximum inter-marker distance of 53 kb. The remaining 12 SNPs did not have any nearby SNPs associated at the 0.5 q-value threshold. Using an LD approach (r²>=0.2) to define the regions, resulted in nineteen regions of associations as defined by an r² greater than 0.2. (See Tables 5a, 5b, and FIG. 8.) Regions associated with those SNPs include several known genes including CLEC4A, CSMD1, DNAH3, EBF2, ELMO1, ENPP6, KBTBD9, MSRB3, MYO5B, and TSC2.

6.2.2 Genes within the Chromosomal Regions

Linkage disequilibrium refers to the co-inheritance of alleles (e.g. alternative nucleotides) at two or more different SNPs at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are referred to as being in “linkage equilibrium”. In contrast, LD refers to any non-random genetic association between allele(s) at two or more different SNP sites. Thus, if a particular SNP site is useful for diagnosing pulmonary disease (e.g. has a significant statistical association with the condition and/or is recognized as a causative polymorphism for the condition), then a skilled artisan will recognize that other SNP sites, which are in LD with this SNP site, would also be useful for diagnosing the condition. For example, SNPs that are not causative polymorphisms, but are in LD with one or more causative SNPs are also useful for diagnosing the pulmonary disease. Thus, SNPs that are in LD with causative polymorphisms are also useful as diagnostic markers of pulmonary diseases. Useful LD SNPs can be selected from among the SNPs disclosed in Tables 5a, 5b, 7, 8, and FIG. 8 for example. Below are particular embodiments of the present disclosure incorporating LD analysis.

TABLE 5a

| | | | HWE p- | | Missing | | Analysis with | Min p- | Min q- | Case/Control | |
|---|---|---|---|---|---|---|---|---|---|---|---|
| Chr | base pair | SNP rs# | value | MAF | freq. | Gene/Region | q < .50 | value | value | p-value | q |
| 1 | 65200064 | rs4915675 | 0.78 | 0.25 | 0 | | Smoke Exposure | 0.000022 | 0.41 | 0.3672 | 0.98 |
| 2 | 23628257 | rs4665609 | 0.03 | 0.46 | 0 | KBTBD9 | Case-Control | 7.58E−07 | 0.39 | 7.581E−07 | 0.39 |
| 2 | 168246597 | rs2029084 | 0.38 | 0.28 | 0 | | Smoke Exposure | 0.000016 | 0.38 | 0.4947 | 0.98 |
| 4 | 185283504 | rs7689305 | 1 | 0.31 | 0 | ENPP6 | Age Decline | 2.33E−07 | 0.12 | 0.05214 | 0.95 |
| 6 | 158871063 | rs7772700 | 0.91 | 0.43 | 0 | | Smoke Exposure | 8.69E−06 | 0.32 | 0.5002 | 0.98 |
| 7 | 37326734 | rs6947058 | 0.73 | 0.33 | 0 | ELMO1 | Smoke Exposure | 0.000027 | 0.46 | 0.7889 | 1 |
| 8 | 3992429 | rs6989761 | 0.82 | 0.35 | 0 | CSMD1 | Smoke Exposure | 7.35E−06 | 0.32 | 0.1784 | 0.97 |
| 8 | 3999687 | rs6999426 | 0.79 | 0.25 | 0 | CSMD1 | Smoke Exposure | 0.000019 | 0.38 | 0.4097 | 0.98 |
| 8 | 3999872 | rs2002195 | 0.89 | 0.25 | 0 | CSMD1 | Smoke Exposure | 0.000015 | 0.38 | 0.3644 | 0.98 |
| 8 | 25950860 | rs17818981 | 0.71 | 0.29 | 0 | EBF2 | Smoke Exposure | 9.38E−06 | 0.32 | 0.02084 | 0.93 |
| 9 | 13667557 | rs688703 | 0.51 | 0.26 | 0.003 | | Smoke Exposure | 4.15E−06 | 0.32 | 0.2316 | 0.97 |
| 9 | 27605794 | rs504532 | 0.8 | 0.30 | 0 | ch9 cluster 1 | Smoke Exposure | 6.6E−06 | 0.32 | 0.7012 | 0.99 |
| 9 | 27611563 | rs10968015 | 0.35 | 0.26 | 0 | ch9 cluster 1 | Smoke Exposure | 8.29E−06 | 0.32 | 0.7986 | 1 |
| 9 | 27621390 | rs10812628 | 0.43 | 0.26 | 0 | ch9 cluster 1 | Smoke Exposure | 5.58E−06 | 0.32 | 0.9467 | 1 |
| 9 | 77521024 | rs795035 | 0.32 | 0.29 | 0.030 | ch9 cluster 2 | Smoke Exposure | 5.98E−06 | 0.32 | 0.548 | 0.98 |
| 9 | 77522623 | rs2990413 | 0.02 | 0.49 | 0 | ch9 cluster 2 | Smoke Exposure | 0.000022 | 0.41 | 0.04676 | 0.95 |
| 12 | 8179670 | rs17728942 | 1 | 0.17 | 0 | CLEC4A | Smoke Exposure | 0.000015 | 0.38 | 0.2037 | 0.97 |
| 12 | 64253454 | rs4237904 | 0.11 | 0.25 | 0 | ch12 cluster | Smoke Exposure | 0.000019 | 0.38 | 0.01371 | 0.92 |
| 12 | 64266091 | rs10784478 | 0.11 | 0.25 | 0 | ch12 cluster | Smoke Exposure | 0.000019 | 0.38 | 0.01371 | 0.92 |
| 12 | 64292755 | rs2248625 | 0.21 | 0.24 | 0 | ch12 cluster | Smoke Exposure | 3.54E−06 | 0.32 | 0.03133 | 0.94 |
| 12 | 64301834 | rs7976914 | 0.21 | 0.24 | 0 | ch12 cluster | Smoke Exposure | 3.54E−06 | 0.32 | 0.03133 | 0.94 |
| 13 | 72001650 | rs12866475 | 0.79 | 0.26 | 0.003 | | Smoke Exposure | 0.0000044 | 0.32 | 0.1633 | 0.97 |
| 13 | 85735283 | rs12584999 | 0.34 | 0.20 | 0 | | Smoke Exposure | 0.000027 | 0.46 | 0.2124 | 0.97 |
| 13 | 102392437 | rs9300771 | 0.73 | 0.34 | 0.003 | ch13 cluster | Smoke Exposure | 0.000017 | 0.38 | 0.554 | 0.98 |
| 13 | 102400495 | rs1019893 | 0.73 | 0.34 | 0.003 | ch13 cluster | Smoke Exposure | 0.000017 | 0.38 | 0.554 | 0.98 |
| 13 | 102402430 | rs7985500 | 0.73 | 0.34 | 0.003 | ch13 cluster | Smoke Exposure | 0.000017 | 0.38 | 0.554 | 0.98 |
| 16 | 2073902 | rs30259 | 0.78 | 0.11 | 0 | TSC2 | fev1/fvc | 2.44E−06 | 0.42 | 0.005327 | 0.91 |
| 16 | 20871819 | rs12051478 | 0.7 | 0.07 | 0 | DNAH3 | Smoke Exposure | 0.000013 | 0.38 | 0.5138 | 0.98 |
| 16 | 20882570 | rs3743696 | 0.65 | 0.06 | 0 | DNAH3 | Smoke Exposure | 0.000017 | 0.38 | 0.3956 | 0.98 |
| 18 | 45674781 | rs1787321 | 0.88 | 0.23 | 0 | MYO5B | Smoke Exposure | 1.9E−06 | 0.32 | 0.1158 | 0.96 |
| 18 | 45728495 | rs1787291 | 0.11 | 0.15 | 0 | MYO5B | Smoke Exposure | 7.58E−06 | 0.32 | 0.0001544 | 0.63 |
| 18 | 45732121 | rs1787585 | 0.11 | 0.15 | 0 | MYO5B | Smoke Exposure | 7.58E−06 | 0.32 | 0.0001544 | 0.63 |
| 18 | 45732228 | rs8097868 | 0.16 | 0.15 | 0 | MYO5B | Smoke Exposure | 3.99E−06 | 0.32 | 0.00003823 | 0.56 |

TABLE 5b

| | | Chro- | | Up SNP | Up SNP | Down SNP | Down SNP | Interval | |
|---|---|---|---|---|---|---|---|---|---|
| Region | SNP | mosome | SNPbp | (r2 >= 0.2) | position (bp) | (r2 >= 0.2) | position (bp) | Size | RefSeq Genes |
| 1 | rs4915675 | 1 | 65200064 | rs6676160 | 64994430 | rs1338516 | 65287192 | 292762 | JAK1, RAVER2 |
| 2 | rs4665609 | 2 | 23628257 | rs1432268 | 23623939 | rs605750 | 23696195 | 72256 | NA |
| 3 | rs2029084 | 2 | 168246597 | rs2390601 | 168223608 | rs6433006 | 168271898 | 48290 | NA |
| 4 | rs7689305 | 4 | 185283504 | rs6819770 | 185253393 | rs1921564 | 185315070 | 61677 | ENPP6 |
| 5 | rs7772700 | 6 | 158871063 | rs341127 | 158785645 | rs9364973 | 158895704 | 110059 | TMEM181, TULP4 |
| 6 | rs6947058 | 7 | 37326734 | rs3847014 | 37326813 | rs10251451 | 37329120 | 2307 | ELMO1 |
| 7 | rs6989761 | 8 | 3992429 | rs12674985 | 3945429 | rs1714708 | 4048612 | 103183 | CSMD1 |
| 7 | rs6999426 | 8 | 3999687 | rs17068917 | 3937389 | rs1714708 | 4048612 | 111223 | CSMD1 |
| 7 | rs2002195 | 8 | 3999872 | rs17068917 | 3937389 | rs1714708 | 4048612 | 111223 | CSMD1 |
| 8 | rs17818981 | 8 | 25950860 | rs1008975 | 25960681 | rs6557880 | 25976212 | 15531 | EBF2 |
| 9 | rs688703 | 9 | 13667557 | rs2382402 | 13606003 | rs717605 | 13726965 | 120962 | NA |
| 10 | rs504532 | 9 | 27605794 | rs10968015 | 27611563 | rs10812628 | 27621390 | 9827 | NA |
| 10 | rs10968015 | 9 | 27611563 | rs17779794 | 27600116 | rs10812628 | 27621390 | 21274 | NA |
| 10 | rs10812628 | 9 | 27621390 | rs17779794 | 27600116 | rs536635 | 27617362 | 17246 | NA |
| 11 | rs795085 | 9 | 77521024 | rs4745437 | 77497877 | rs6560469 | 77640744 | 142867 | NA |
| 11 | rs2990413 | 9 | 77522623 | rs1328548 | 77492323 | rs2149385 | 77529588 | 37265 | NA |
| 12 | rs17728942 | 12 | 8179670 | rs1990476 | 8166003 | rs1133104 | 8182389 | 16386 | CLEC4A |
| 13 | rs4237904 | 12 | 64253454 | rs2245225 | 64216921 | rs2453269 | 64339959 | 123038 | NA |
| 13 | rs10784478 | 12 | 64266091 | rs2245225 | 64216921 | rs2453269 | 64339959 | 123038 | NA |
| 13 | rs2248625 | 12 | 64292755 | rs2255312 | 64226306 | rs2453269 | 64339959 | 113653 | NA |
| 13 | rs7976914 | 12 | 64301834 | rs2255312 | 64226306 | rs2453269 | 64339959 | 113653 | NA |
| 14 | rs12866475 | 13 | 72001650 | rs17833217 | 72000549 | rs12866475 | 72001650 | 1101 | NA |
| 15 | rs12584999 | 13 | 85735283 | rs2184263 | 85625744 | rs1939662 | 85747575 | 121831 | NA |
| 16 | rs9300771 | 13 | 102392437 | rs701546 | 102378362 | rs6491721 | 102465179 | 86817 | NA |
| 16 | rs1019893 | 13 | 102400495 | rs701546 | 102378362 | rs6491721 | 102465179 | 86817 | NA |
| 16 | rs7985500 | 13 | 102402430 | rs701546 | 102378362 | rs6491721 | 102465179 | 86817 | NA |
| 17 | rs30259 | 16 | 2073902 | rs28537973 | 20308579 | rs13335638 | 2076625 | 38046 | TSC2 |
| 18 | rs12051478 | 16 | 20871819 | rs7498905 | 20601568 | rs2112494 | 20952870 | 351302 | ACSM1, ACSM3, |
| | | | | | | | | | DCUN1D3, DNAH3, |
| | | | | | | | | | EXOD1, LOC81691, |
| | | | | | | | | | LYRM1, THUMPD1 |
| 18 | rs3743696 | 16 | 20882570 | rs231921 | 20569262 | rs13337676 | 21002350 | 433088 | ACSM1, ACSM3, |
| | | | | | | | | | DCUN1D3, DNAH3, |
| | | | | | | | | | EXOD1, LOC81691, |
| | | | | | | | | | LYRM1, THUMPD1 |
| 19 | rs1787321 | 18 | 45674781 | rs8083571 | 45472119 | rs8097868 | 45732228 | 260109 | ACAA2, MYO5B |
| 19 | rs1787291 | 18 | 45728495 | rs869013 | 45515353 | rs17659350 | 45787095 | 271742 | ACAA2, MYO5B |
| 19 | rs1787585 | 18 | 45732121 | rs869013 | 45515353 | rs17659350 | 45787095 | 271742 | ACAA2, MYO5B |
| 19 | rs8097868 | 18 | 45732228 | rs869013 | 45515353 | rs17659350 | 45787095 | 271742 | ACAA2, MYO5B |

Table 5a shows the top SNPs for GWAS with q-values <0.5, and Table 5b shows the assignment of those SNPs to 19 different chromosomal regions defined by an LD where r²>0.2 between the SNPs in Table 5a and flanking SNPs. For the purpose of this disclosure, “Smoke Exposure” is also called “CPD×Age.”

### CSMD1

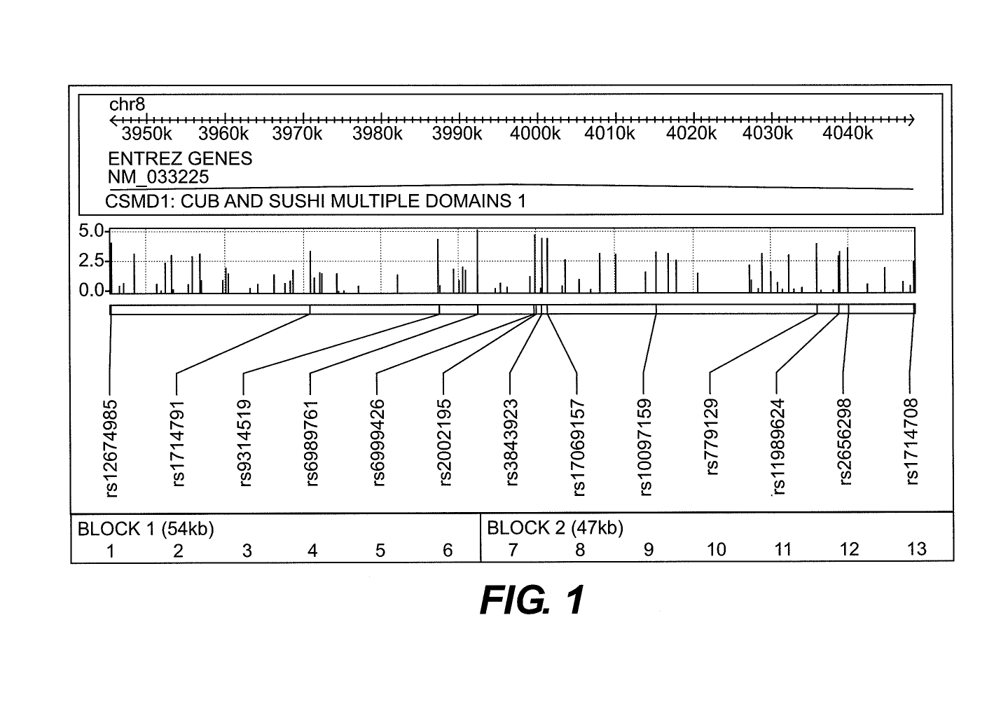

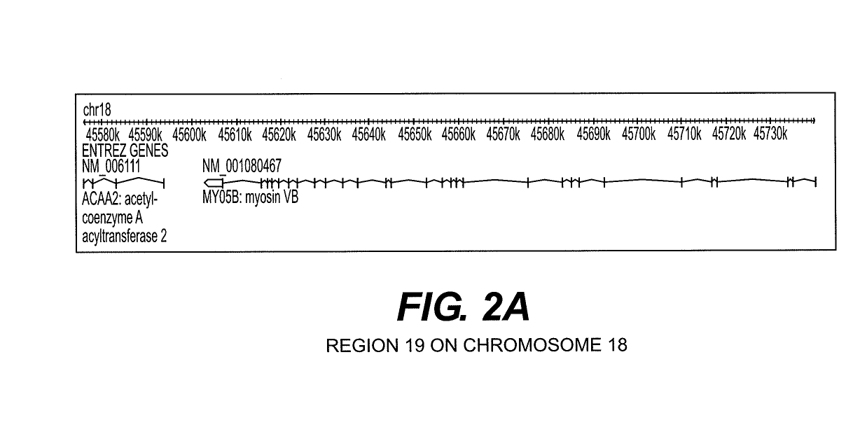

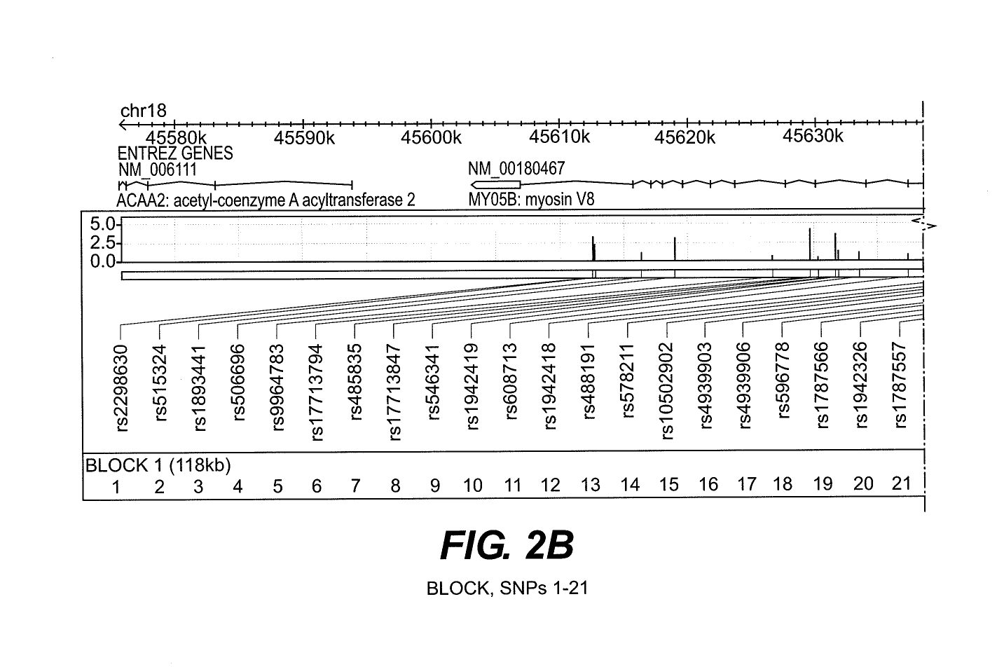

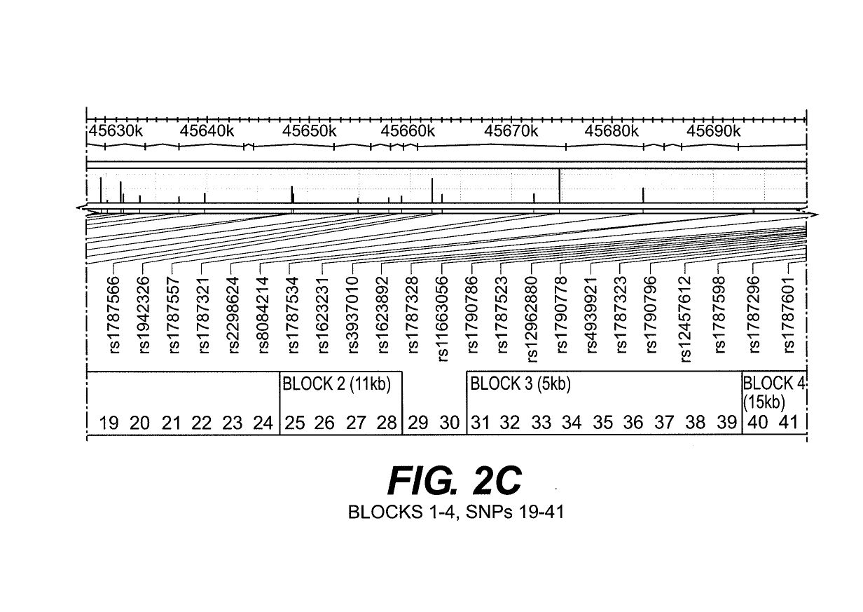

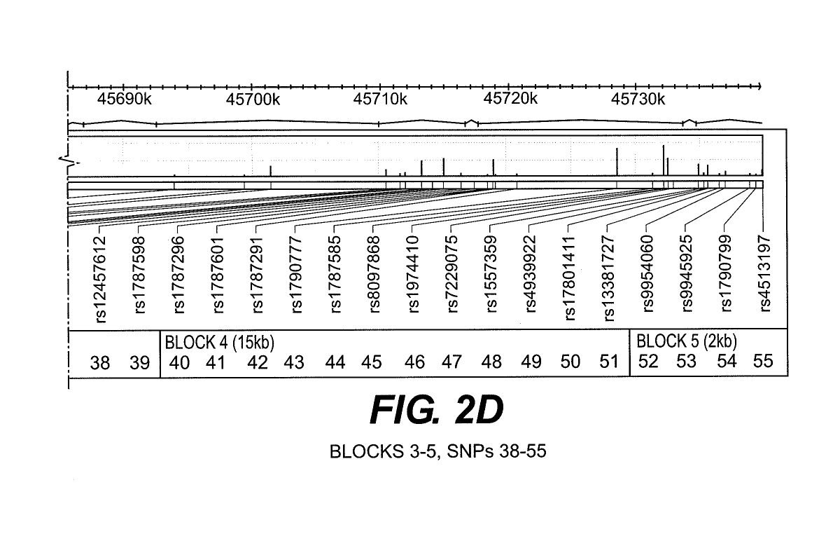

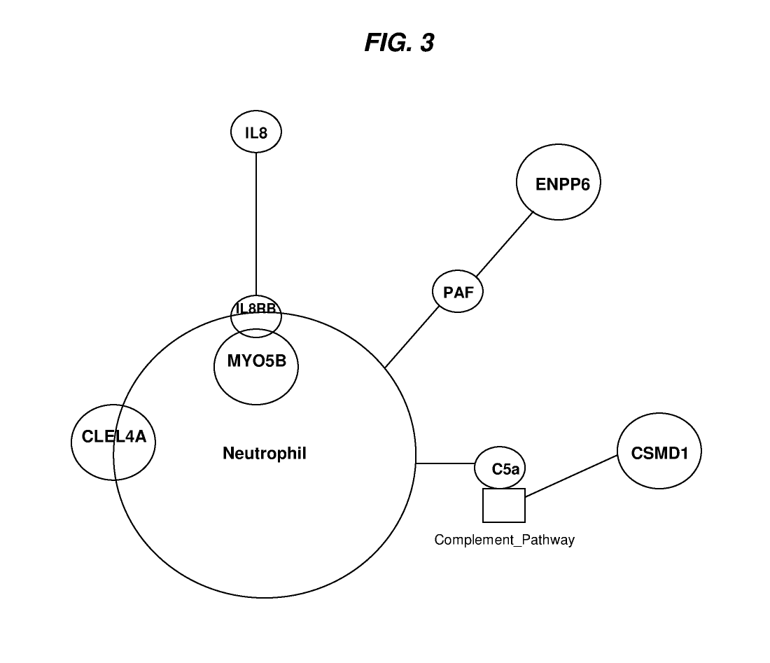

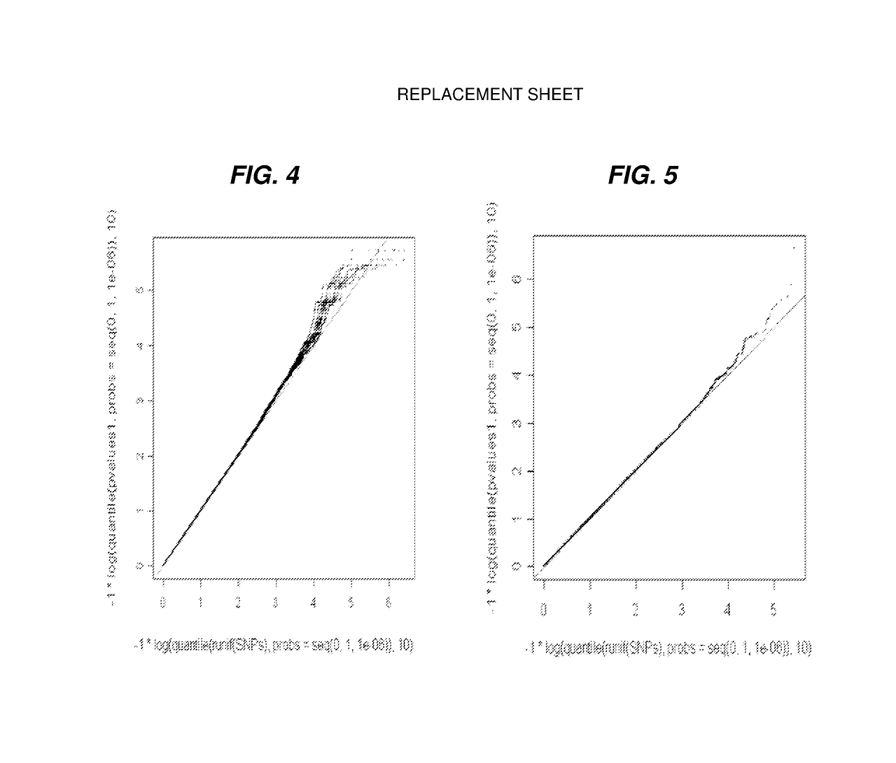

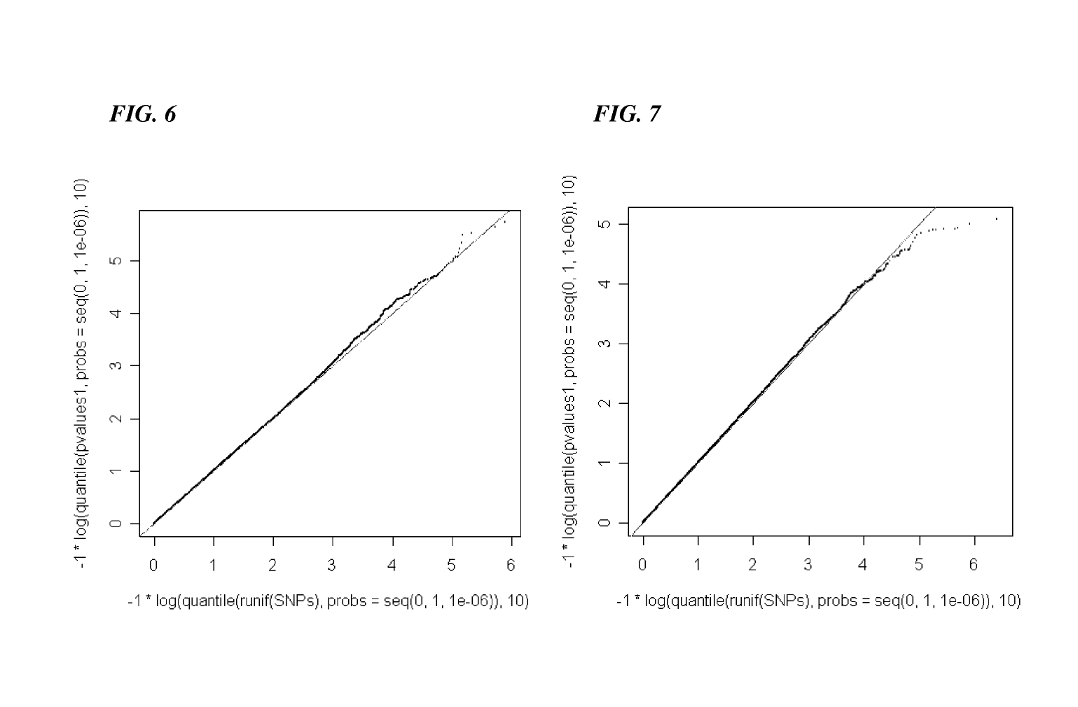

The LD patterns in the regions for selected SNPs that clustered in genes were examined. For CSMD1 (CUB and Sushi multiple domains 1) on chromosome 8p, three SNPs in a 7.4 kilobase (kb) region had p-values less than 1.9×10⁻⁵ and individual q-values between 0.32 and 0.38. Further examination of the association identified three additional associated markers in a 103 kb region that had a minimum q-value of 0.75 within 50 kb of the core and contained 80 markers in all. A total of 9, 22, and 29 significant SNPs were found in this region (p-value=0.0001, 0.001, and 0.01, respectively). Linkage disequilibrium and association results for a portion of the region are shown in FIG. 1 for markers with p-values ≦0.0005. Two haplotype blocks extending over a total of 103 kb were observed using a solid spline of LD block algorithm, with the three most significant markers in an area where the D′ does not fall below 0.9. Although the extended area of association appears to contain multiple blocks, the associated markers are in elevated LD with each other, suggesting that they probably represent a single association signal.

Recently CSMD1 has been shown to inactivate the classic complement pathway (Kraus et al. 2006, J. Immunol. 176 (7):4419-4430). Recently, COPD has been shown to be in part an autoimmune disease with anti-elastin autoantibodies being detected in COPD patients (Lee et al. 2007, Nat. Med. 13 (5):567-569). Smoking-induced recurrent infections or autoimmunity may lead to a persistent activation of the complement system. Genetic variability in the regulation of the complement system as suggested by the association with CSMD1 provided herein could explain in part the different risk of COPD development or progression given a certain exposure level.

### MYO5B

Four SNPs in MYO5B had p-values less than 7.58×10⁻⁶. MYO5B, which encodes the Myosin VB protein, a large gene extending over 372 kb with a total of 123 SNPs tested. A large section (˜210 kb) of the gene did not show any significantly associated markers. Three additional associated markers were found in a 164 kb region that had a minimum q-value of 0.75 and was within 50 kb of the core. A total of 6, 9, and 19 of the 55 SNPs in this region were significant (p-values less than 0.0001, 0.001, and 0.01, respectively). Three SNPs in MYO5B were also significantly associated with COPD using the less powerful case-control categories (p-values <1×10⁻⁴). When the core of the MYO5B association was restricted to a 7.4 kb region, the four most significantly associated SNPs in MYO5B covered 57.4 kb. The extended 164 kb region was primarily within the MYO5B gene but extends into the gene ACAA2. Examination of LD across the 164 kb region revealed at least two different distinct signals not in high LD (D′˜0.42) with each other.

### DNAH3

DNAH3 is a large gene extending over 226 kb. A total of 33 SNPs were tested in DNAH3, and two SNPs had p-values ≦1.7×10⁻⁵. One additional SNP, rs2301620, had a q-value less than 0.75 (p-value 8.96×10⁻⁵). These three SNPs covered 15.2 kb, and examination of LD showed they were in high LD with marker-to-marker D′ greater than 0.99 and minimum D′ of 0.82.

DNAH3 encodes the dynein axonemal heavy chain 3, which is used in the assembly of cilia. Axonemal dyneins are microtubule-associated motor protein complexes necessary for cilia and flagella function. Cilia are critically important in the clearance of material including mucus and particulate matter from the lung. DNAH3 is also known as DLP3, DNAHC3B, Hsadhc3, FLJ31947, FLJ43919, FLJ43964, and DKFZp434N074.

### ENPP6

The most significant GWAS association was with rs7689305 in the gene ENPP6 for the Age Decline BLUP (p-value=2.33×10⁻⁷, q-value=0.12). An additional three SNPs in ENPP6 had p-values less than 0.000005 (q-value ˜0.53). The four associated SNPs were in a single 30 kb region of high LD (minimum D′=0.94, r=0.32) Fig. These SNPs also showed association with the FEV1/FVC ratio (p-value 0.000076, q-value 0.95) but not case-control status.

ENPP6 encodes an ectonucleotide pyrophosphatase/phosphodiesterase and is in the ether lipid pathway. The enzyme has Phospholipase C (PLC) activity and can act on lysoplasmalogen and platelet activating factor (PAF) (Sakagami et al. 2005, J. Biol. Chem. 280 (24):23084-23093). PAF is a powerful mediator of hypersensitivity and inflammation and a direct activator of neutrophils that are thought to be an important in COPD. While not wishing to be bound by theory, if genetic variation led to an increased or decreased abundance or activity of ENPP6, the amount or duration of PAF would be altered thereby potentially influencing neutrophil behavior and activity. A related gene ENPP2 has shown evidence for involvement in mouse lung function (Ganguly et al. 2007, Physiol Genomics. 31 (3):410-421) and expression levels are predictive of lung cancer survival (Lu et al. 2006, PLoS. Med. 3 (12):e467). ENP6 is also known as NPP6 and MGC33971.

### Methionine Sulfoxide Reductases (MSRA)

A cluster of significant SNPs near MSRB3, which encodes methionine sulfoxide reductase B3, was observed. Evidence for association with MSRA (p-value 0.0000069, q-value of 0.61) was also observed. Methionine sulfoxide reductase is an enzyme that reverses oxidative protein damage by reducing methionine sulfoxide back to methionine. It may play an important role in protection from oxidative stress.

6.2.3 Other Genes

Associations at an FDR of 0.5 for a single SNP were observed in genes CLEC4A, EBF2, and ELMO1 for the Pack-years decline BLUP, in KBTBD9 for case versus control status, and in TSC2 for the ratio FEV₁/FVC.

CLEC4A encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The encoded type 2 transmembrane protein may play a role in inflammatory and immune response. Multiple transcript variants encoding distinct isoforms have been identified for this gene. This gene is closely linked to other CTL/CTLD superfamily members on chromosome 12p13 in the natural killer gene complex region. CLEC4A is also known as DCIR, LLIR, DDB27, CLECSF6, and HDCGC13P.

EBF2 belongs to the conserved Olf/EBF family (see MIM 164343) of helix-loop-helix transcription factors. EBF2 is also known as COE2, OE-3, EBF-2, O/E-3, and FLJ11500.

ELMO1 encodes a protein that interacts with the dedicator of cyto-kinesis 1 protein to promote phagocytosis and effect cell shape changes. Similarity to a C. elegans protein suggests that this protein may function in apoptosis and in cell migration. Alternative splicing of this gene results in multiple transcript variants encoding different isoforms. ELMO1 is also known as CED12, CED-12, ELMO-1, KIAA0281, and MGC126406.

More than half of the significant SNPs were found in intergenic regions, often in clusters. Two clusters were observed on chromosome 9, including three SNPs covering 15.6 kb at megabase 27.6 and two SNPs covering 1.6 kb at megabase 77.5 Mb. Another group of four associated SNPs covering 48 kb was found on chromosome 12 around 64.2 Mb. This cluster was 103 kb from the gene MSRB3 that encodes methionine sulfoxide reductase B3. Three SNPs within 10 kb were observed near 102.4 Mb on chromosome 13. However, these represent SNPs in perfect LD and may not be a cluster as their allele frequencies and p-values were identical. Additional significant singleton SNPs are listed in FIG. 8 and in Tables 5a, 5b and 8.

TABLE 6
NCBI Accession and GI No. of Homo sapiens genes coding sequences of CLEC4A,
CSMD1, DNAH3, EBF2, ELMO1, ENPP6, KBTBD9, MSRB3, MYO5B, and TSC2:

| | Accession No. Version |
|---|---|
| | and/or GI No. |
| | (Nucleotide and Amino |
| Gene Name/Info. | Acid SEQ ID NOs): |
| CLEC4A: C-type lectin domain family 4, member A [Homo sapiens] | Variants: |
| Other Aliases: HDCGC13P, CLECSF6, DCIR, DDB27, LLIR | NM_016184.3/GI:148536834 |
| Other Designations: C-type (calcium dependent, carbohydrate- | (SEQ ID NO: 1 SEQ ID NO: 2); |
| recognition domain) lectin, superfamily member 6; C-type lectin | NM_194447.2/GI:148536835 |
| DDB27; C-type lectin domain family 4 member A; C-type lectin | (SEQ ID NO: 3 SEQ ID NO: 4); |
| superfamily member 6; dendritic cell immunoreceptor; lectin-like | NM_194448.2/GI:148536837 |
| immunoreceptor | (SEQ ID NO: 5 SEQ ID NO: 6); |
| Chromosome: 12; Location: 12p13 | NM_194450.2/GI:148536838 |
| Annotation: Chromosome 12, NC_000012.11 (8276228 . . . 8291203) | (SEQ ID NO: 7 SEQ ID NO: 8); |
| CSMD1: CUB and Sushi multiple domains 1 [Homo sapiens] | NM_033225.5/GI:259013212 |
| Other Aliases: UNQ5952/PRO19863, KIAA1890 | SEQ ID NO: 9 SEQ ID NO: 10); |
| Other Designations: CUB and sushi domain-containing protein 1; | |
| CUB and sushi multiple domains protein 1 | |
| Chromosome: 8; Location: 8p23.2 | |
| Annotation: Chromosome 8, NC_000008.10 (2792875 . . . 4852328, | |
| complement) | |
| DNAH3: dynein, axonemal, heavy chain 3 [Homo sapiens] | NM_017539.1/GI:24308168 |
| Other Aliases: DKFZp434N074, DLP3, DNAHC3B, FLJ31947, | (SEQ ID NO: 11 SEQ ID NO: 12); |
| FLJ43919, FLJ43964, Hsadhc3 | |
| Other Designations: axonemal beta dynein heavy chain 3; axonemal | |
| dynein, heavy chain; ciliary dynein heavy chain 3; dnahc3-b; dynein | |
| heavy chain 3, axonemal; dynein, axonemal, heavy polypeptide 3 | |
| Chromosome: 16; Location: 16p12.3 | |
| Annotation: Chromosome 16, NC_000016.9 (20944476 . . . 21170762, | |
| complement) | |
| EBF2: early B-cell factor 2 [Homo sapiens] | NM_022659.2/GI:113930702 |
| Other Aliases: COE2, EBF-2, FLJ11500, O/E-3, OE-3 | (SEQ ID NO: 13 SEQ ID NO: 14); |
| Other Designations: Collier, Olf and EBF 2; OLF-1/EBF-LIKE 3; | |
| metencephalon-mesencephalnon-olfactory transcription factor 1; | |
| transcription factor COE2 | |
| Chromosome: 8; Location: 8p21.2 | |
| Annotation: Chromosome 8, NC_000008.10 (25701573 . . . 25902392, | |
| complement) | |
| ELMO1: engulfment and cell motility 1 [Homo sapiens] | Variants: |
| Other Aliases: CED-12, CED12, ELMO-1, KIAA0281, MGC126406 | NM_014800.9/GI:86787650 |
| Other Designations: OTTHUMP00000128236; ced-12 homolog 1; | (SEQ ID NO: 15 SEQ ID NO: 16); |
| engulfment and cell motility protein 1; protein ced-12 homolog | NM_001039459.1/GI:86788139 |
| Chromosome: 7; Location: 7p14.1 | (SEQ ID NO: 17 SEQ ID NO: 18); |
| Annotation: Chromosome 7, NC_000007.13 (36893961 . . . 37488511, | NM_130442.2/GI:86788141 |
| complement) | (SEQ ID NO: 19 SEQ ID NO: 20); |
| ENPP6: ectonucleotide pyrophosphatase/phosphodiesterase 6 | NM_153343.3/GI:195539377 |
| [Homo sapiens] | (SEQ ID NO: 21 SEQ ID NO: 22); |
| Other Aliases: UNQ1889/PRO4334, MGC33971, NPP6 | |
| Other Designations: B830047L21Rik; E-NPP 6; NPP-6; | |
| ectonucleotide pyrophosphatase/phosphodiesterase family member 6 | |
| Chromosome: 4; Location: 4q35.1 | |
| Annotation: Chromosome 4, NC_000004.11 | |
| (185009859 . . . 185139114, complement) | |
| KBTBD9: kelch-like 29 (Drosophila) [Homo sapiens] | NM_052920.1/GI:256818753 |
| Other Aliases: KLHL29, KIAA1921 | (SEQ ID NO: 23 SEQ ID NO: 24); |
| Other Designations: OTTHUMP00000216456; kelch repeat and | |
| BTB (POZ) domain containing 9; kelch repeat and BTB domain- | |
| containing protein 9; kelch-like protein 29 | |
| Chromosome: 2; Location: 2p24.1 | |
| Annotation: Chromosome 2, NC_000002.11 (23608298 . . . 23931483) | |
| MSRB3: methionine sulfoxide reductase B3 [Homo sapiens] | Variants: |
| Other Aliases: UNQ1965/PRO4487, DKFZp686C1178, FLJ36866 | NM_001031679.2/GI:301336160 |
| Other Designations: methionine-R-sulfoxide reductase B3; | (SEQ ID NO: 25 SEQ ID NO: 26); |
| methionine-R-sulfoxide reductase B3, mitochondrial | |
| Chromosome: 12; Location: 12q14.3 | |
| Annotation: Chromosome 12, NC_000012.11 (65672423 . . . 65860687) | |
| MYO5B: myosin VB [Homo sapiens] | NM_001080467.2/GI:239915992 |
| Other Aliases: KIAA1119 | (SEQ ID NO: 27 SEQ ID NO: 28); |
| Other Designations: MYO5B variant protein; myosin-Vb | |
| Chromosome: 18; Location: 18q21 | |
| Annotation: Chromosome 18, NC_000018.9 (47349156 . . . 47721451, | |
| complement) | |
| TSC2: tuberous sclerosis 2 [Homo sapiens] | Variants: |
| Other Aliases: FLJ43106, LAM, TSC4 | NM_000548.3/GI:116256351 |
| Other Designations: OTTHUMP00000198394; tuberin; tuberous | (SEQ ID NO: 29 SEQ ID NO: 30); |
| sclerosis 2 protein | NM_001077183.1/GI:116256349 |
| Chromosome: 16; Location: 16p13.3 | (SEQ ID NO: 31 SEQ ID NO: 32); |
| Annotation: Chromosome 16, NC_000016.9 (2097990 . . . 2138713) | NM_001114382.1/GI:167412123 |
| | (SEQ ID NO: 33 SEQ ID NO: 34); |

Unless otherwise indicated, the nucleic acids listed or set forth in Table 6 by NCBI accession or GI number include: nucleic acids having the sequences recited under the Accession and/or GI number, the complement of those sequences; and either or both strands (if double stranded). Where the identifiers recite a genomic sequence, the mRNA (or cDNAs thereof) are also available in the databases of the NCBI and are considered part of this disclosure.

6.3 Summary

In summary, four different BLUPs measuring individual differences in processes involved in COPD were analyzed and SNPs having an association with four lung function decline BLUPs are provided herein. Thirty-three SNPs significant at a FDR of less than 50% are provided herein. The minimum q-value of 0.12 was found in ENPP6. Clusters of SNPs meeting the FDR cut off were found in genes CSMD1, MYO5B, and DNAH3. Additionally, SNPs below the critical FDR were found in the genes CLEC4A, EBF2, ELMO1, and TSC2.

Multiple SNPs in MYO5B were associated with the Pack-years decline BLUP and importantly the categorical analysis based on case-control status. This allows other groups with samples but without longitudinal data sets, and therefore not able to generate comparable BLUPs, to directly replicate the findings in this study. Two distinct signals were also discovered in MYO5B that were only in modest LD with each other and therefore represent separate results. Multiple SNPs indicate results are not technical errors. The combination of MYO5B having multiple independent association signals, makes a useful marker for the methods and kits provided herein.

The sample size for the investigation described herein was modest for a GWAS of a complex trait. However, the investigation described herein has the advantage of having long-term repeated measures. These measures enabled the modeling of decline in lung function and the separation of the effects of age, baseline lung function, and cigarette smoking. The resulting phenotypic analyses produced more homogenous quantitative outcomes. Quantitative measures are inherently more powerful and decreasing heterogeneity further increases power. One approach is to analyze cigarette smoking-related BLUP-based SNPs for associations contingent on or as an interaction with a measure of smoking such as pack-years.

### 7.0 Example 2 Replication Data Analysis and Modeling

7.1 Materials and Methods

7.1.1 Study Design and Subjects

The COPD Biomarker Discovery Study (CBD) was a cross-sectional study at the University of Utah to identify novel diagnostic, prognostic or therapeutic biomarkers of COPD in adult current or former cigarette smokers. Male and female self-reported cigarette smokers, aged 45 years or older, with at least 10 pack-years smoking history were recruited from the University Health Sciences Network of local clinics and hospitals and from community physician offices. COPD was diagnosed in 300 subjects according to the Global Initiative for Chronic Obstructive Lung Disease (GOLD) spirometric guidelines as having a ratio of forced expiratory volume in 1 second (s) (FEV₁) to forced vital capacity (FVC)<0.70 (Rabe et al. 2007). The control group included 425 sex- and age-matched (using 10-year bands), current or former cigarette smokers, without apparent lung disease who had FEV₁/FVC≧0.70, and were recruited from the same clinical settings. Individuals who had recent exacerbation of COPD, uncontrolled angina, hypertension, or allergy to albuterol, and females who were pregnant or lactating were excluded. Demographic variables, respiratory symptoms and medical history, tobacco use history, and concomitant medications were assessed. Pack-years were calculated as (maximum average number of cigarettes smoked daily over total smoking history/20)×(total years smoking). Body weight and height were measured. Spirometry was performed with a rolling seal spirometer by certified pulmonary function technicians according to Amer. Thoracic Society guidelines (Miller et al. 2005, Euro. Resp. J. 26:319-338). Measurements of FEV₁ and FVC were made before and at least 20 min after inhaled bronchodilator administration (albuterol 180 μg). The FEV₁/FVC ratio was calculated for each subject from the highest post-bronchodilator values of FEV₁ and FVC. A blood sample was collected for assessment of carboxyhemoglobin (COHb) and complete blood cell counts.

7.1.2 Blood Sample Collection and Processing

Whole blood samples were obtained from each subject by venipuncture using 10 mL EDTA Vacutainer® tubes (BD, Franklin Lakes, N.J., USA). White blood cells were separated from the whole blood samples and used as a source of DNA.

DNA was extracted from white blood cells, purified (Puregene Kit, Gentra Systems, Inc, Minneapolis, Minn.), and stored at −70° C. In 601 case and control samples genotyping was performed in accordance with manufacturer-recommended procedures using the Infinium II HumanHap 1M SNP array (Illumina, San Diego, Calif.) on a BeadStation. Robotic liquid handling stations were used for sample handling. The HumanHap 1M array assays N tagging SNPs selected from Phases I and II of the HapMap Project. Genotypes were called using BeadStudio genotyping module version 3.2.32. The mean call rate of arrays in the analysis was 0.998, and arrays with a fail rate above 0.980 were repeated.

7.2. Association Analysis

All replication association analyses were performed in PLINK. The minimum allowable SNP and individuals genotyping success rates were 0.9. The minimum allowable observed SNP minor allele frequency (MAF) was 0.05. Additional quality control steps included screening of SNPs with a Hardy-Weinberg Equilibrium test p-value <1×10⁻⁶.

7.2.1 Stratification

Subjects were predominantly Caucasian, but there were a small number of subjects from other ethnic groups. Population substructure could result in false positive findings if the subgroups differed in allele frequencies, prevalence of COPD, or quantitative measures of lung function decline. A variety of methods is available to detect population substructure and correct for its potential confounding effects. Sullivan et al. (Sullivan et al. 2008, Mol. Psychiatry. 13 (6):570-584) performed an extensive evaluation of multiple statistical methods to avoid false positive findings in GWAS due to such genetic subgroups. They concluded that the principal components and multi-dimensional scaling (MDS) approaches were very similar and superior to other approaches. MDS was used for practical reasons as it can be implemented in PLINK (Purcell et al. 2007).

Input data for the MDS approach were the genome-wide average proportion of alleles shared identically by state (IBS) between any two individuals. Somewhat analogous to principal component analysis, the first MDS dimension of a (genetic) similarity matrix captures the maximal variance in the genetic similarity, the second dimension must be orthogonal to the first and captures the maximum amount of residual genetic similarity, and so on. A one-dimension solution was the best-fitting model to account for the genetic similarity among subjects in this sample.

7.3 Results

7.3.1 GWAS Replication

A total of 601 assays (225 Cases, 367 Controls, 9 missing) from the PLINK output, each with 1,072,821 SNPs, was performed and passed quality control. A total of 6 subjects were eliminated as ancestry outliers. After filtering by fail rate, minimum minor allele frequency and HWE, 751,305 SNPs were analyzed for association with four phenotypes (COPD, Percent Predicted FVC, Percent Predicted FEV1, and the ratio (FEV₁/FVC). In each analysis, smoking (pack years) and the first and second MDS ancestry dimensions were treated as covariates in a linear model for the quantitative traits and in a logistic model for the qualitative disease status (COPD). In addition, age and sex were included as covariates in the logistic model. Results focused on the results within the 19 associated regions previously described that contain genes that have already been identified in Example 1, including CLEC4A, CSMD1, DNAH3, EBF2, ELMO1, ENPP6, KBTBD9, MSRB3, MYO5B, ENPP6 and TSC2. See, e.g., Tables 5b and 6 and in FIG. 8.

Analysis of the data in this example confirms the association of a number of genomic regions with pulmonary diseases such as COPD. This analysis, however, which employed a population that was on average older, had poorer lung function, was thinner, and smoked more, indicated that the more common alleles found in the SNPS identified in region 19 correlate with case rather than control status, which is the opposite of the finding in Example 1. That alleles associated with the same disease/phenotype may appear to flip without changes in the linkage disequlibrium has been describe in the art. See, e.g., Clarke et al., Genetic Epidemiology 34:266-274 (2010); Lin et al., The Amer. J. of Human Genetics 80: 531-538 (2007); and Zaykin et al. The Amer. J. of Human Genetics 82: 794-800 (2008). Multiple regression analysis employing analysis data and covariates from both Examples 1 and 2 is consistent with that finding, that region 19 contains genetic variations that are significantly associated with a predisposition for COPD and risk factors and spirometric indicators for developing COPD (e.g., pack years FEV₁/FVC). Hence, individuals with genetic variations in that region may benefit from monitoring, prophylactic treatment and/or treatment. Analysis of genetic variations in region 19, particularly in conjunction with other genetic variations, described herein, also leads to an ability to diagnose a pulmonary disease, to predict the development of a pulmonary disease, to determine the probability of its development, and/or to predict its ultimate severity.

799 SNPs across the 19 genomic regions for the 4 phenotypes (total 3196 tests) were tested. Among those tests, 301 tests yielded FDR values <0.5. In Table 7, below, the top 20 results across phenotypes are presented. In the text below, the proportion of SNPs in each region yielding uncorrected p-values <0.05 is presented.

TABLE 7

| SNP | Region | Phenotype | P-value | FDR |
|---|---|---|---|---|
| rs1787321 | 19 | percent predicted | 1.44E−04 | 0.09 |
| | | FEV1 | | |
| rs657424 | 19 | FEV₁/FVC Ratio | 1.36E−04 | 0.09 |
| rs1787566 | 19 | FEV₁/FVC Ratio | 1.92E−04 | 0.09 |
| rs1787321 | 19 | FEV₁/FVC Ratio | 4.45E−05 | 0.09 |
| rs1787291 | 19 | FEV₁/FVC Ratio | 1.97E−04 | 0.09 |
| rs1787585 | 19 | FEV₁/FVC Ratio | 1.86E−04 | 0.09 |
| rs8097868 | 19 | FEV₁/FVC Ratio | 1.21E−04 | 0.09 |
| rs485835 | 19 | FEV₁/FVC Ratio | 3.11E−04 | 0.124 |
| rs490697 | 19 | FEV₁/FVC Ratio | 3.71E−04 | 0.124 |
| rs546341 | 19 | FEV₁/FVC Ratio | 3.88E−04 | 0.124 |
| rs2679726 | 19 | FEV₁/FVC Ratio | 5.80E−04 | 0.168 |
| rs8097868 | 19 | COPD | 9.43E−04 | 0.236 |
| rs10945546 | 5 | percent predicted | 9.59E−04 | 0.236 |
| | | FEV1 | | |
| rs485835 | 19 | COPD | 3.37E−03 | 0.251 |
| rs546341 | 19 | COPD | 3.07E−03 | 0.251 |
| rs657424 | 19 | COPD | 2.45E−03 | 0.251 |
| rs1787566 | 19 | COPD | 2.50E−03 | 0.251 |
| rs1787321 | 19 | COPD | 3.17E−03 | 0.251 |
| rs1787291 | 19 | COPD | 1.22E−03 | 0.251 |

COPD is defined as FEV₁/FVC less than 0.70

Region 1—Chromosome 1: 64994430 Base Pairs (bp)-65287192 Base Pairs (bp)

Region 1 (see e.g., NCBI Contig Accession Numbers: NW_001838579.2/GI:157811766; NW_921351.1/GI:88950243 and NT_032977.9) contains 74 SNPs in Phase1B. Of those, 14 were significant (nominal p-values <0.05) for association with FVC, 12 were significant (nominal p-values <0.05) for association with FEV1 and 1 for FEV1/FVC ratio.

Region 2—Chromosome 2: 23623939 bp-23696195 bp

Region 2 (see e.g., NCBI Contig Accession Numbers: NT_022184.15/GI:224515010 and NW_001838768.1) contains 26 SNPs in Phase 1B. One SNP was significant (nominal p-value <0.05) for an association with FVC and one SNP was significant at a nominal p-value of 0.05 for FEV1/FVC ratio.

Region 3—Chromosome 2: 168223608 bp-168271898 bp

Region 3 (see e.g., NCBI Contig Accession Numbers: NW_001838860.1/GI:157696421, NT_005403.17 and NW_921585.1) yielded no significant results in 20 Phase1B SNPs at a p-value of 0.05 across phenotypes.

Region 4—Chromosome 4: 185253393 bp-185315070 bp

Region 4 (see e.g., NCBI Contig Accession Numbers: NT_016354.19/GI:224514665, NW_001838921.1/GI:157696482 and NW_922217.1/GI:88981534) yielded 1 significant result (nominal p-value <0.05) for FEV1 among 25 Phase1B SNPs.

Region 5—Chromosome 6: 158785645 bp-158895704 bp

Region 5 (see e.g., NCBI Contig Accession Numbers: NT_025741.15/GI:224514841, NW_001838991.2 and NW_923184.1) contains 41 SNPs, 13 were significant (nominal p-values <0.05) for COPD, 9 for FVC, 11 for FEV1, and 2 were significant (nominal p-values <0.05) for FEV1/FVC ratio.

Region 6—Chromosome 7: 37326813 bp-37329120 bp

Region 6 (see e.g., NCBI Contig Accession Numbers: NT_007819.17/GI:224514859, NW_001839003.1/GI:157696564, NW_923240.1/GI:89025910 and NT_079592.2/GI:89026958) contains 4 SNPs none of which were significant at p<0.05.

Region 7—Chromosome 8: 3937389 bp-4048612 bp

Region 7 (see e.g., NCBI Contig Accession Numbers: NW_001839109.2/GI:157812071 and NW_923840.1/GI:89028496) contains 109 SNPs, 7 of which were significant (nominal p-values <0.05) for COPD, 12 of which were significant (nominal p-values <0.05) for FVC and 1 of which was significant for FEV1 (nominal p-values <0.05).

Region 8—Chromosome 8: 25960681 bp-25976212 bp

Region 8 (see e.g., NCBI Contig Accession Numbers: NT_167187.1/GI:224514765, NT_167187.1/GI:224514765 and NT_167187.1/GI:224514765) comprises 7 SNPs none of which were significant across the association tests.

Region 9—Chromosome 9: 13606003 bp-13726965 bp

Region 9 (see e.g., NCBI Contig Accession Numbers: NW_001839149.2 GI:157812089, NT_008413.18 GI:224514694 and NW_924062.1 GI:89030318) comprises 39 SNPs, 1 of which was significant (nominal p-values <0.05) for COPD and 1 of which was significant (nominal p-values <0.05) for FEV1/FVC ratio.

Region 10—Chromosome 9: 27600116 bp-27621390 bp

Region 10 (see e.g., NCBI Contig Accession Numbers: NT_008413.18/GI:224514694, NW_001839149.2/GI:157812089 and NW_924062.1/GI:89030318) contains 17 SNPs none of which were significant at a nominal p-value of 0.05.

Region 11—Chromosome 9: 77492323 bp-77640744 bp

Region 11 (see e.g., NCBI Contig Accession Numbers: NT_008470.19/GI:224514751, NW_001839221.1/GI:157696782 and NW_924484.1/GI:89030471) contains 61 Phase1B SNPs, 3 of which were significant (nominal p-values <0.05) for COPD, 1 for FVC, and 1 was significant (nominal p-values <0.05) for FEV1/FVC ratio.

Region 12—Chromosome 12: 8166003 bp-8182389 bp

Region 12 (see e.g., NCBI Contig Accession Numbers NW_001838051.1/GI:157696928, NT_009714.17/GI:224514867 and NW_925295.1/GI:89035948) contains 14 SNPs, 3 of which were significant (nominal p-values <0.05) for FVC at a p-value<0.05.

Region 13—Chromosome 12: 64216921 bp-64339959 bp

Region 13 (see e.g., NCBI Contig Accession Numbers NW_001838060.2/GI:157812191, NW_925395.1/GI:89036563 and NT_029419.12/GI:224514900) contains 29 SNPs, 1 of which was significant (nominal p-values <0.05) for FEV1 at a p-value<0.05.

Region 14—Chromosome 13: 72000549 bp-72000549 bp

Region 14 (see e.g., NCBI Contig Accession Numbers NT_024524.14/GI:224514830, NW_001838081.1 GI:157696958 and NW_925506.1/GI:89037138) contains 1 SNP which was not significant at a p-value<0.05.

Region 15—Chromosome 13: 85625744 bp-85747575 bp

Region 15 (see e.g., NCBI Contig Accession Numbers: NT_024524.14/GI:224514830, NW_001838083.1/GI:157696960, NW_001838084.2/GI:157812203, NW_925506.1/GI:89037138, and NW_925517.1/GI:89037217) contains 26 SNPs, 2 of which were significant (nominal p-values <0.05) for COPD, 11 of which were significant (nominal p-values <0.05) for FVC, 7 of which were significant (nominal p-values <0.05) for FEV1 and 4 for FEV1/FVC ratio.

Region 16—Chromosome 13: 102378362 bp-102465179 bp

Region 16 (see e.g., NCBI Contig Accession Numbers: NT_009952.14/GI:37544901, NW_001838084.2/GI:157812203 and NW_925517.1/GI:89037217) contains 41 SNPs, 12 of which were significant (nominal p-values <0.05) for association with FVC and 10 of which were significant (nominal p-values <0.05) for FEV1.

Region 17—Chromosome 16: 2038579 bp-2076625 bp

Region 17 (see e.g., NCBI Contig Accession Numbers: NT_010393.16/GI:224514941, NW_001838339.2/GI:157812280 and NW_926018.1/GI:89040669) contains 13 SNPs, 1 of which was significant (nominal p-values <0.05) for COPD, FVC and FEV1/FVC ratio.

Region 18—Chromosome 16: 20569262 bp-21002350 bp

Region 18 (see e.g., NCBI Contig Accession Numbers: NT_010393.16/GI:224514941, NW_001838381.1/GI:157697600 and NW_926184.1/GI:89040724) contains 112 SNPS, 1 of which was significant (nominal p-values <0.05) for COPD, 18 for FEV1 and 16 (nominal p-values <0.05) for FEV1/FVC ratio.

Region 19—Chromosome 18: 45472119 bp-45787095 bp

Region 19 (see e.g., NCBI Contig Accession Numbers: NW_001838468.1 GI:157697806, NT_010966.14/GI:224514957 and NW_927106.1/GI:89047489) contains 140 SNPs, 35 of which were significant (nominal p-values <0.05) for COPD, 15 of which were significant for FVC, 39 of which were significant (nominal p-values <0.05) for FEV1, and 45 were significant (nominal p-values <0.05) for FEV1/FVC ratio.

### 8.0 Consolidated Listing of SNPs

Table 8 provides a consolidated listing of SNPs by the region in which they are found along with the sequences of those SNPs and the polymorphism shown.

While the technology has been particularly shown and described with reference to specific illustrative embodiments, it should be understood that various changes in form and detail may be made without departing from the spirit and scope of the technology.

TABLE 8

| Region | SNP | Chromosome | SEQUENCE | SEQ ID NO. |
|---|---|---|---|---|
| 1 | rs1338516 | 1 | TTCATTTGCTTTTGAACTTGCAGAAA[C/T]GGGAGTGAAGTGATTTCTGATTTTT | 35 |
| 1 | rs4915675 | 1 | AAAGCATTTGACAAGGGCTCCACGCA[A/G]GAATTAGCTCTCTTCAGGGTCCTGG | 36 |
| 1 | rs6676160 | 1 | CCTTCATGATTAGAGTCAAGTTTTAT[A/G]TCTTTAGCAGGAACATCACAAGGTG | 37 |
| 2 | rs1432268 | 2 | GTAGCCAGCACACAGTAAGTGCCCAG[A/G]AAGTGTTCGCTTTCCGTAGTAGAAG | 38 |
| 2 | rs4665609 | 2 | TCCCCAGGCGATGCTGTGGCTACTGG[A/C]CTATGGACCACATTTTGAGTAGGGA | 39 |
| 2 | rs605750 | 2 | TCCCAGCCTGTTAGTGCCTAGTTCAC[A/G]CTCCCAACTTTTCCTGAACACCTAC | 40 |
| 3 | rs2029084 | 2 | CTGAAAACAGCCTGCACTACTGACAA[A/C]GGCTTTGTGTATCCTCTTTAGATTT | 41 |
| 3 | rs2390601 | 2 | GCATTTAAATAAAATCTGGATAGTTG[C/T]TGTTAATCAAGGCCATGTAGATTTG | 42 |
| 3 | rs6433006 | 2 | TGACAGCTAGTGCACACCTTTCAGCC[A/G]TGGTAGTGAGCCACCTTGAGAGTGG | 43 |
| 4 | rs1921564 | 4 | TCAGAAATGGCTGGCCTTCACATCTC[A/G]CGAGAAGGTAGAGGATATGTCCATC | 44 |
| 4 | rs6819770 | 4 | GCTTTTAGTGTTACAGGAACCTGTGA[C/T]GGAGGCCTCTGTTAATGGACAGAAT | 45 |
| 4 | rs7689305 | 4 | TTGACCAAGGGTTCAGAGAACTTCTG[A/G]GCAACACTGTATGTGTAGAGAACTG | 46 |
| 5 | rs341127 | 6 | AAAGACAAAGGTACTGATGAGATACT[A/G]TGGCTTCCAAAATAGAAATCTTTTG | 47 |
| 5 | rs7772700 | 6 | TGTGATGCTACGTAAAATCAGGGAAA[C/T]GGGGCTGTTTCTGAGTAAGCTACAA | 48 |
| 5 | rs9364973 | 6 | ACCAATCTGAATAGAATTTAAGGGTC[C/T]ATGCTAGATCTTACCATGAAGACAC | 49 |
| 5 | rs10945546 | 6 | TTTTAAGTACAGGAGGGAGCCAAAGC[A/G]CACACACACTACAGGACAATGCCTG | 50 |
| 6 | rs10251451 | 7 | AAAAGCAGGAATTTTTTCAGAATAAC[C/T]TAGAGGATTAGGCAGTTACCACATT | 51 |
| 6 | rs3847014 | 7 | CTGTCCCTTGAGAACAAGGCATCTTA[A/G]TTCATTTCTGTAGCCTTCCCCACCC | 52 |
| 6 | rs6947058 | 7 | TAGATGTAATTACTCCCTCTGTGTAC[G/T]TAGCACATTAAATTAATAACTTCTG | 53 |
| 7 | rs12674985 | 8 | CTTTTCTAAGCCTTAGTCTCATCAAC[C/T]ATAAAATGGATTAAAAATGGGTATC | 54 |
| 7 | rs17068917 | 8 | TATATTATGACCATATTATGACACTC[C/T]TATCTTTGGTAAAATGATAATTAAG | 55 |
| 7 | rs1714708 | 8 | TGGTTCCTCTCCTGGCCATTTGTAAG[C/T]AGGGATCACACACACACAAACATAC | 56 |
| 7 | rs2002195 | 8 | ATTCCAAGTCTATTGACAATAATACA[A/G]AATGTTATATTGAAAATTAAGTGGG | 57 |
| 7 | rs6989761 | 8 | TGATTGCCTTTGTGCTCCCACCACAA[C/T]CTGTTCCTGTCTCCATTAGAGCCCT | 58 |
| 7 | rs6999426 | 8 | TTATGCAAGTAAGGCTAATATCCCCG[G/T]AAGATATGAATATCACTGATCACAG | 59 |
| 8 | rs1008975 | 8 | ATGCAGGTTTTACGGAGAATTTCGGT[C/T]CCAGCAAAAACTGATCACCTGGAGT | 60 |
| 8 | rs17818981 | 8 | TGTCTCTAATTTCAAACTCAAATAAG[C/T]GCACAGCATGGTGGCTTTTGTTTTG | 61 |
| 8 | rs6557880 | 8 | GCCACACCTGGCCTTTTTCCTCCCCA[A/G]TCAACTGGTCATAAGGAATCACCCA | 62 |
| 9 | rs2382402 | 9 | TTTCCTGAGGTTGTCCAGCCAAAATA[C/T]ATTACAACATGTTGTTATGGACTGG | 63 |
| 9 | rs688703 | 9 | TGACTCTCAGCAACATACCATAAGCA[A/G]GGACTCTGCTTTCTTTCCCACTTAT | 64 |
| 9 | rs717605 | 9 | TTAAGTCATGGCATGCCTTGCATGCT[G/T]GTGTATATGGTTTTGCCTTATGAAC | 65 |
| 10 | rs10812628 | 9 | AGAGCATTGACACTTGTAGGGCAAGC[A/G]TGAAGCAGGGAGAGCAGCCAGGAGT | 66 |
| 10 | rs10968015 | 9 | AATTAAAAGTATTATAACCAGTGGGG[A/G]TAAGGATGCAGTAAAACAGACATGT | 67 |
| 10 | rs17779794 | 9 | AAAAGCTGTCTCTCGTTTTCCTGGAG[C/T]TGAGAATTTTCATTCAAAGCATCTT | 68 |
| 10 | rs504532 | 9 | CCAAGATACAAAGATGTAGATTTTTC[C/T]ACCAGTAAAACAAAGATTCACTAGG | 69 |
| 10 | rs536635 | 9 | CAGTAAGCAACAAAAACCCGTTCTCT[A/G]GAATACCTCTAGGCTGTCTCTCTTA | 70 |
| 11 | rs1328548 | 9 | CCATCATTTGGGTTTGAGCAGCACTC[C/T]GCCAGTGACCTTCTGATATACTATA | 71 |
| 11 | rs2149385 | 9 | CTAAAGAAAGTACAACTGGCCAATTT[C/T]AATTTAAGTTCTGCATTTAAAAAAT | 72 |
| 11 | rs2990413 | 9 | GATTTATAATAAAAGGTAAGTGACGG[C/T]CTTTTGGTTCACAGTATTTCTCAGC | 73 |
| 11 | rs4745437 | 9 | ATAAGGTACAATGGACCAGCAAACAA[C/T]AGAATGTCTTAAAATTATGGGAAAA | 74 |
| 11 | rs6560469 | 9 | CCATAAGCCAAAATTCAGCTGGTTAC[A/G]TCAATTGCAGGTATCACCAATGGGG | 75 |
| 11 | rs795085 | 9 | TACCAACCTGGATTTAAAAGGTACCT[A/C]TTCCTAAGTAACTTATCCAGCATCT | 76 |
| 12 | rs1133104 | 12 | TACTGGAGGCCCCCATTGTGCACACA[G/T]GGAGAGAACATGAGTCTCTCTTAAT | 77 |
| 12 | rs17728942 | 12 | TGTATATCTCTCTTGGCTAAGAAGGA[A/G]GTTTTTGTTACTTTGGGATATTTGC | 78 |
| 12 | rs1990476 | 12 | TTTCTTCATCCTGCTTGGGCTCTGAC[A/T]CTCCATGCAGGTCCTCCATCCCCCA | 79 |
| 13 | rs10784478 | 12 | TCCAAGAAACTAAGAACTACTGCAAA[A/G]GGGATAGATTCTTCCAGAATACAAA | 80 |
| 13 | rs2245225 | 12 | TGATGTCAAGACTCCTTCCTCCCTGC[A/G]TTCTTTTCTTCTCTGGGACAGGCTA | 81 |
| 13 | rs2255312 | 12 | TCTGTTTAGCTCATGGTCGGGAACTC[A/G]GGCCCTTGAAAATGAGGCACTGTTC | 82 |
| 13 | rs2453269 | 12 | AGAAAGTAGAACACTGTCACTGCAGA[C/T]AACCAAGCTGAAAAATGAGCATCTC | 83 |
| 13 | rs4237904 | 12 | ATTGGGAGCTGAATATTGGCATAGTA[G/T]CAAAGTATCTCCCTGCCAAATACTT | 84 |
| 13 | rs7976914 | 12 | GACATTTCACCTTCATTAGAACAGCG[A/C]CTTAAATCATGTTTGTCTTAGGAAA | 85 |
| 14 | rs12866475 | 13 | CATGCCTAATGCAGATTTTTCCAAAA[C/T]ACGTGATAATGCATACTGTATATTA | 86 |
| 14 | rs17833217 | 13 | AATTCATTATGCAAACAGAAATCTGC[A/G]AACAATAAGACAGGCAATAGCAAGT | 87 |
| 15 | rs12584999 | 13 | AATGGTCATAGTATAATTTAGCCTAG[A/G]TATAGCTTGACATCATTTATTTGAA | 88 |
| 15 | rs1939662 | 13 | TGCCTCTCTGAGTTACTGGCTATCTT[A/G]TTTTTCTATTTTTAATTTGTGTTTA | 89 |
| 15 | rs2184263 | 13 | ATTGCGCTGCCACATTATCATGGCCA[C/T]AGTGTGTGTAGGCAATAGAAATTTT | 90 |
| 16 | rs1019893 | 13 | AAACCGATGTGTTCGATTTAGACTTA[A/G]CGTTCATTTTGAGTTACATTTTTTA | 91 |
| 16 | rs6491721 | 13 | CCACTTCAAAATTCACTTCAGGATGT[A/C/G]TTTCCTGGGGAAGCTTTTCTAGA | 92 |
| | | | TC | |
| 16 | rs701546 | 13 | TTCAACAATAGTAACAATTCAAGAAA[C/T]AAGTGCGATAGACACAAAATGCTAT | 93 |
| 16 | rs7985500 | 13 | CGTATCAGGGATGAAACAGGGCCTGG[A/C]AGGCAGCTGCAACACCGAGTAGCGG | 94 |
| 16 | rs9300771 | 13 | CCTGAGGAGTTTATTTAGCAGAAGGT[A/G]GACATATTAGATTGCATGATACTTA | 95 |
| 17 | rs13335638 | 16 | CACTGGCCAGGCACCAGAGGACGTGG[C/T]CCCCGCAGGCCCCCAGAGCCCCTGG | 96 |
| 17 | rs28537973 | 16 | TGCTCAGATGTCCCCATTCCTGTTTC[C/G]TTTGCACAGAGGGGTTTTCTGGTGC | 97 |
| 17 | rs30259 | 16 | CCCCCAAGTTCAGAGCCAGTTCCCAG[A/G]GTGCAGGCACACCCACGCAGAGCCC | 98 |
| 18 | rs12051478 | 16 | GGCCAGCCTTAAAGAAATGACCACTC[A/G]TATTTCCAAGGGTGTAATGATAAAT | 99 |
| 18 | rs13337676 | 16 | CTTTTAGATTTGTGGCTTCCATTTCG[C/T]TTGAAACCACAGTAGCAACCCCTTT | 100 |
| 18 | rs2112494 | 16 | GTCTTGCCGCCCATGGGGTCTCCTAC[A/G]ATCATATAGCCATGTCTCACCAGCA | 101 |
| 18 | rs231921 | 16 | AACGTGCAGCGGCCCTACAGGGAAAT[C/T]CCCAACAAAAATTAATTTAAAATTG | 102 |
| 18 | rs3743696 | 16 | ATTTCCTTCTTCTGTTTCATGATGCC[A/G]ATGGTCAGGAGGAGAGAGAAGAGTA | 103 |
| 18 | rs7498905 | 16 | ACTGTAAATGGATCTAGCCAAAAAAT[A/G]GGTGGACACTGCTTTACACACATTT | 104 |
| 19 | rs17659350 | 18 | AAGATCAAGCCCTTCCTCCTCATTTC[C/T]GGGTGGTGCCACCGGGAGAGAGAGT | 105 |
| 19 | rs1787291 | 18 | ATCTTTTATATTCTTATAAACACAAA[C/T]GAGTAGGTGTGATTTCCAAGGTAAC | 106 |
| 19 | rs1787321 | 18 | GGAGCAGGGAATCTCTATGCCCTGAT[A/G]CTCAGGTTTGGGGCAAAGCTCAGGA | 107 |
| 19 | rs1787585 | 18 | CTGTGACAACTTATAGGGCCAGAAAA[C/T]TCTGTTGTCTCAGTAGAAGTTTGTC | 108 |
| 19 | rs8083571 | 18 | GCGCCATAGGCAGACAAACAGAAGAT[A/G]TCAATGTCCTTTCTGGGAAGAGCCC | 109 |
| 19 | rs8097868 | 18 | CACTTCCATCTACTCTCTTTCCCTGT[A/G]CCTTGGGGCTCCTCCCTATGCCACC | 110 |
| 19 | rs869013 | 18 | CCTTATGCTTTCATGATGAATGAAAC[C/T]GAGAGGACCAACTTGGGATTTTTCC | 111 |
| 19 | rs657424 | 18 | CACACAGCACTTCACTGCCTCCCTCT[A/C]TATCAGCCATCTGTCTCCTCTCTCC | 112 |
| 19 | rs1787566 | 18 | TAATAAATAGCAAAAACATTTTTTAA[A/G]AACTTTCTTCGCACTTTTTTTTTTT | 113 |
| 19 | rs485835 | 18 | AGATTGGAAGTTTAATCCTGACACTC[A/C]ATAGCATGGAGTGAGGACCTTGGGG | 114 |
| 19 | rs490697 | 18 | GCAGTTGGAGGTGACCAGTGCGGCCC[A/G]TGGGCAGCCGTCAGAAATGCGCCAG | 115 |
| 19 | rs546341 | 18 | AAGATTAATCCAGGCCAGGCTTTGAC[G/T]CCTGTCTTTGAGAGCTCTGACATCT | 116 |
| 19 | rs2679726 | 18 | TAAGTTTTAGACCTTTTAGTATCCAC[A/G]TAAAATTGACATCAAATGAAAATTG | 117 |
| 19 | rs485835 | 18 | AGATTGGAAGTTTAATCCTGACACTC[A/C]ATAGCATGGAGTGAGGACCTTGGGG | 119 |
| 19 | rs546341 | 18 | AAGATTAATCCAGGCCAGGCTTTGAC[G/T]CCTGTCTTTGAGAGCTCTGACATCT | 120 |

Unless otherwise indicated, the nucleic acids listed or set forth in Table 8 include: nucleic acids having the sequences recited in the table and/or their complement and/or both strands (e.g., as a double stranded sequence).

1.-59. (canceled)

60. An apparatus comprising a device with a surface having a plurality of locations, each location comprising a nucleic acid having a single nucleotide polymorphism (SNP) recited in Table 8 bound thereto;
wherein said apparatus comprises from 4 to 85 nucleic acids having different SNPs recited in Table 8 bound thereto; and
wherein a nucleic acid comprising at least one of the SNPs recited in Table 8 is not bound to a location on the device.

61. The apparatus of claim 60, wherein said surface has bound thereto nucleic acids comprising from 6 to 85 different SNPs recited in Table 8.

62. The apparatus of claim 61, wherein said surface has bound thereto at least 6 nucleic acids comprising different SNPs recited in Table 7.

63. The apparatus of claim 60, wherein the nucleic acid having a SNP recited in Table 8 is an amplification product of genomic nucleic acid or cDNA.

64. The apparatus of claim 63, wherein different nucleic acids are polymerase chain reaction, oligonucleotide ligation, or ligase chain reaction amplification products.

65. The apparatus of claim 60, wherein said nucleic acids are detectably labeled.

66. A composition comprising nucleic acid probes or primers for detection of 4 to 85 of the Single Nucleotide Polymorphisms (SNPs) in Table 8.

67. The composition of claim 66, wherein the composition comprises nucleic acids for detection of 6 to 85 of the SNPs in Table 8.

68. The composition of claim 66, wherein the composition is an array of nucleic acids, wherein the nucleic acids are each bound to a solid support.

69. The composition of claim 68, wherein the solid support comprises a surface with a plurality of locations.

70. The composition of claim 66, wherein the nucleic acids are detectably labeled.

71. The composition of claim 70, wherein the detectable label is selected from the group consisting of isotope label or fluorescent label.

72. The composition of claim 66, wherein the composition comprises a single base extension and fluorescence resonance energy transfer primer.

73. The composition of claim 66, wherein the nucleic acids comprise a peptide nucleic acid.

74. The composition of claim 66, wherein the composition comprises nucleic acids for detection of one or more SNPs from at least two of chromosomal regions 1-19.

75. The composition of claim 66, comprising nucleic acids for detection of at least 4 SNPs in Table 7.

76. The composition of claim 66, comprising nucleic acids for detection of each of the SNPs in Table 7.

77. A composition comprising a plurality of solid supports, each solid support comprising a nucleic acid having a sequence including a SNP recited in Table 8;
wherein said composition has bound thereto nucleic acids comprising from 4 to 85 different SNPs recited in Table 8; and
wherein a nucleic acid comprising the sequence of at least one of the SNPs recited in Table 8 is not bound to a location on the device.

78. The composition of claim 77, wherein said solid supports are the individual beads of a bead array.


SEQUENCE LISTING (34 sequences)

SEQ ID NO: 1 - DNA, 1284 bp, Homo sapiens
ctgtgattctcactatactggtcctgaggaaagggcttctgtgaactgcggtttttagtttttattgtggttcttagttctcatgagacccctcttgaggatatgtgcctatctggtgcctctgctctccactagttgagtgaaaggaaggaggtaatttaccaccatgtttggttcctgtttataagatgttttaagaaagatctgaaacagattttctgaagaaagcagaagctctcttcccattatgacttcggaaatcacttatgctgaagtgaggttcaaaaatgaattcaagtcctcaggcatcaacacagcctcttctgcagcttccaaggagaggactgcccctcacaaaagtaataccggattccccaagctgctttgtgcctcactgttgatatttttcctgctattggcaatctcattctttattgcttttgtcattttctttcaaaaatattctcagcttcttgaaaaaaagactacaaaagagctggttcatacaacattggagtgtgtgaaaaaaaatatgcccgtggaagagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccacattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcaaa

SEQ ID NO: 2 - protein, 237 aa, Homo sapiens
MTSEITYAEVRFKNEFKSSGINTASSAASKERTAPHKSNTGFPKLLCASLLIFFLLLAISFFIAFVIFFQKYSQLLEKKTTKELVHTTLECVKKNMPVEETAWSCCPKNWKSFSSNCYFISTESASWQDSEKDCARMEAHLLVINTQEEQDFIFQNLQEESAYFVGLSDPEGQRHWQWVDQTPYNESSTFWHPREPSDPNERCVVLNFRKSPKRWGWNDVNCLGPQRSVCEMMKIHL

SEQ ID NO: 3 - DNA, 1167 bp, Homo sapiens
ctgtgattctcactatactggtcctgaggaaagggcttctgtgaactgcggtttttagtttttattgtggttcttagttctcatgagacccctcttgaggatatgtgcctatctggtgcctctgctctccactagttgagtgaaaggaaggaggtaatttaccaccatgtttggttcctgtttataagatgttttaagaaagatctgaaacagattttctgaagaaagcagaagctctcttcccattatgacttcggaaatcacttatgctgaagtgaggttcaaaaatgaattcaagtcctcaggcatcaacacagcctcttctgcagttttctttcaaaaatattctcagcttcttgaaaaaaagactacaaaagagctggttcatacaacattggagtgtgtgaaaaaaaatatgcccgtggaagagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccacattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcaaa

SEQ ID NO: 4 - protein, 198 aa, Homo sapiens
MTSEITYAEVRFKNEFKSSGINTASSAVFFQKYSQLLEKKTTKELVHTTLECVKKNMPVEETAWSCCPKNWKSFSSNCYFISTESASWQDSEKDCARMEAHLLVINTQEEQDFIFQNLQEESAYFVGLSDPEGQRHWQWVDQTPYNESSTFWHPREPSDPNERCVVLNFRKSPKRWGWNDVNCLGPQRSVCEMMKIHL

SEQ ID NO: 5 - DNA, 1068 bp, Homo sapiens
ctgtgattctcactatactggtcctgaggaaagggcttctgtgaactgcggtttttagtttttattgtggttcttagttctcatgagacccctcttgaggatatgtgcctatctggtgcctctgctctccactagttgagtgaaaggaaggaggtaatttaccaccatgtttggttcctgtttataagatgttttaagaaagatctgaaacagattttctgaagaaagcagaagctctcttcccattatgacttcggaaatcacttatgctgaagtgaggttcaaaaatgaattcaagtcctcaggcatcaacacagcctcttctgcagagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccacattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcaaa

SEQ ID NO: 6 - protein, 165 aa, Homo sapiens
MTSEITYAEVRFKNEFKSSGINTASSAETAWSCCPKNWKSFSSNCYFISTESASWQDSEKDCARMEAHLLVINTQEEQDFIFQNLQEESAYFVGLSDPEGQRHWQWVDQTPYNESSTFWHPREPSDPNERCVVLNFRKSPKRWGWNDVNCLGPQRSVCEMMKIHL

SEQ ID NO: 7 - DNA, 1185 bp, Homo sapiens
ctgtgattctcactatactggtcctgaggaaagggcttctgtgaactgcggtttttagtttttattgtggttcttagttctcatgagacccctcttgaggatatgtgcctatctggtgcctctgctctccactagttgagtgaaaggaaggaggtaatttaccaccatgtttggttcctgtttataagatgttttaagaaagatctgaaacagattttctgaagaaagcagaagctctcttcccattatgacttcggaaatcacttatgctgaagtgaggttcaaaaatgaattcaagtcctcaggcatcaacacagcctcttctgcagcttccaaggagaggactgcccctcacaaaagtaataccggattccccaagctgctttgtgcctcactgttgatatttttcctgctattggcaatctcattctttattgcttttgtcaagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccacattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcaaa

SEQ ID NO: 8 - protein, 204 aa, Homo sapiens
MTSEITYAEVRFKNEFKSSGINTASSAASKERTAPHKSNTGFPKLLCASLLIFFLLLAISFFIAFVKTAWSCCPKNWKSFSSNCYFISTESASWQDSEKDCARMEAHLLVINTQEEQDFIFQNLQEESAYFVGLSDPEGQRHWQWVDQTPYNESSTFWHPREPSDPNERCVVLNFRKSPKRWGWNDVNCLGPQRSVCEMMKIHL

SEQ ID NO: 9 - DNA, 14340 bp, Homo sapiens
cccactcgctggctctctctccagctgcctcctctccaggtctctcctggctgcgcgcgctcctctccccgcttctccccctcccgcagcctcgccgccttggtgccttcctgcccggctcggccggcgctcgtccccggccccggccccgccagcccgggtctccgcgctcggagcagctcagccctgcagtggctcgggacccgatgctatgagagggaagcgagccgggcgcccagaccttcaggaggcgtcggatgcgcggcgggtcttgggaccgggctctctctccggctcgccttgccctcgggtgattatttggctccgctcatagccctgccttcctcggaggagccatcggtgtcgcgtgcgtgtggagtatctgcagacatgactgcgtggaggagattccagtcgctgctcctgcttctcgggctgctggtgctgtgcgcgaggctcctcactgcagcgaagggtcagaactgtggaggcttagtccagggtcccaatggcactattgagagcccagggtttcctcacgggtatccgaactatgccaactgcacctggatcatcatcacgggcgagcgcaataggatacagttgtccttccatacctttgctcttgaagaagattttgatattttatcagtttacgatggacagcctcaacaagggaatttaaaagtgagattatcgggatttcagctgccctcctctatagtgagtacaggatctatcctcactctgtggttcacgacagacttcgctgtgagtgcccaaggtttcaaagcattatatgaagttttacctagccacacttgtggaaatcctggagaaatcctgaaaggagttctgcatggaacgagattcaacataggagacaaaatccggtacagctgcctccctggctacatcttggaaggccacgccatcctgacctgcatcgtcagcccaggaaatggtgcatcgtgggacttcccagctcccttttgcagagctgagggagcctgcggaggaaccttacgcgggaccagcagctccatctccagcccgcacttcccttcagagtacgagaacaacgcggactgcacctggaccattctggctgagcccggggacaccattgcgctggtcttcactgactttcagctagaagaaggatatgatttcttagagatcagtggcacggaagctccatccatatggctaactggcatgaacctcccctctccagttatcagtagcaagaattggctacgactccatttcacctctgacagcaaccaccgacgcaaaggatttaacgctcagttccaagtgaaaaaggcgattgagttgaagtcaagaggagtcaagatgctgcccagcaaggatggaagccataaaaactctgtcttgagccaaggaggtgttgcattggtctctgacatgtgtccagatcctgggattccagaaaatggtagaagagcaggttccgacttcagggttggtgcaaatgtacagttttcatgtgaggacaattacgtgctccagggatctaaaagcatcacctgtcagagagttacagagacgctcgctgcttggagtgaccacaggcccatctgccgagcgagaacatgtggatccaatctgcgtgggcccagcggcgtcattacctcccctaattatccggttcagtatgaagataatgcacactgtgtgtgggtcatcaccaccaccgacccggacaaggtcatcaagcttgcctttgaagagtttgagctggagcgaggctatgacaccctgacggttggtgatgctgggaaggtgggagacaccagatcggtcttgtacgtgctcacgggatccagtgttcctgacctcattgtgagcatgagcaaccagatgtggctacatctgcagtcggatgatagcattggctcacctgggtttaaagctgtttaccaagaaattgaaaagggagggtgtggggatcctggaatccccgcctatgggaagcggacgggcagcagtttcctccatggagatacactcacctttgaatgcccggcggcctttgagctggtgggggagagagttatcacctgtcagcagaacaatcagtggtctggcaacaagcccagctgtgtattttcatgtttcttcaactttacggcatcatctgggattattctgtcaccaaattatccagaggaatatgggaacaacatgaactgtgtctggttgattatctcggagccaggaagtcgaattcacctaatctttaatgattttgatgttgagcctcagtttgactttctcgcggtcaaggatgatggcatttctgacataactgtcctgggtactttttctggcaatgaagtgccttcccagctggccagcagtgggcatatagttcgcttggaatttcagtctgaccattccactactggcagagggttcaacatcacttacaccacatttggtcagaatgagtgccatgatcctggcattcctataaacggacgacgttttggtgacaggtttctactcgggagctcggtttctttccactgtgatgatggctttgtcaagacccagggatccgagtccattacctgcatactgcaagacgggaacgtggtctggagctccaccgtgccccgctgtgaagctccatgtggtggacatctgacagcgtccagcggagtcattttgcctcctggatggccaggatattataaggattctttacattgtgaatggataattgaagcaaaaccaggccactctatcaaaataacttttgacagatttcagacagaggtcaattatgacaccttggaggtcagagatgggccagccagttcgtccccactgatcggcgagtaccacggcacccaggcaccccagttcctcatcagcaccgggaacttcatgtacctgctgttcaccactgacaacagccgctccagcatcggcttcctcatccactatgagagtgtgacgcttgagtcggattcctgcctggacccgggcatccctgtgaacggccatcgccacggtggagactttggcatcaggtccacagtgactttcagctgtgacccggggtacacactaagtgacgacgagcccctcgtctgtgagaggaaccaccagtggaaccacgccttgcccagctgcgacgctctatgtggaggctacatccaagggaagagtggaacagtcctttctcctgggtttccagatttttatccaaactctctaaactgcacgtggaccattgaagtgtctcatgggaaaggagttcaaatgatctttcacacctttcatcttgagagttcccacgactatttactgatcacagaggatggaagtttttccgagcccgttgccaggctcaccgggtcggtgttgcctcatacgatcaaggcaggcctgtttggaaacttcactgcccagcttcggtttatatcagacttctcaatttcgtacgagggcttcaatatcacattttcagaatatgacctggagccatgtgatgatcctggagtccctgccttcagccgaagaattggttttcactttggtgtgggagactctctgacgttttcctgcttcctgggatatcgtttagaaggtgccaccaagcttacctgcctgggtgggggccgccgtgtgtggagtgcacctctgccaaggtgtgtggccgaatgtggagcaagtgtcaaaggaaatgaaggaacattactgtctccaaattttccatccaattatgataataaccatgagtgtatctataaaatagaaacagaagccggcaagggcatccaccttagaacacgaagcttccagctgtttgaaggagatactctaaaggtatatgatggaaaagacagttcctcacgtccactgggcacgttcactaaaaatgaacttctggggctgatcctaaacagcacatccaatcacctgtggctagagttcaacaccaatggatctgacaccgaccaaggttttcaactcacctataccagttttgatctggtaaaatgtgaggatccgggcatccctaactacggctataggatccgtgatgaaggccactttaccgacactgtagttctgtacagttgcaacccggggtacgccatgcatggcagcaacaccctgacctgtttgagtggagacaggagagtgtgggacaaaccactaccttcgtgcatagcggaatgtggtggtcagatccatgcagccacatcaggacgaatattgtcccctggctatccagctccgtatgacaacaacctccactgcacctggattatagaggcagacccaggaaagaccattagcctccatttcattgttttcgacacggagatggctcacgacatcctcaaggtctgggacgggccggtggacagtgacatcctgctgaaggagtggagtggctccgcccttccggaggacatccacagcaccttcaactcactcaccctgcagttcgacagcgacttcttcatcagcaagtctggcttctccatccagttctccacctcaattgcagccacctgtaacgatccaggtatgccccaaaatggcacccgctatggagacagcagagaggctggagacaccgtcacattccagtgtgaccctggctatcagctccaaggacaagccaaaatcacctgtgtgcagctgaataaccggttcttttggcaaccagaccctcctacatgcatagctgcttgtggagggaatctgacgggcccagcaggtgttattttgtcacccaactacccacagccgtatcctcctgggaaggaatgtgactggagagtaaaagtgaacccggactttgtcatcgccttgatattcaaaagtttcaacatggagcccagctatgacttcctacacatctatgaaggggaagattccaacagccccctcattgggagttaccagggctctcaggccccagaaagaatagagagtagcggaaacagcctgtttctggcatttcggagtgatgcctccgtgggcctttcagggttcgccattgaatttaaagagaaaccacgggaagcttgttttgacccaggaaatataatgaatgggacaagagttggaacagacttcaagcttggctccaccatcacctaccagtgtgactctggctataagattcttgacccctcatccatcacctgtgtgattggggctgatgggaaaccctcctgggaccaagtgctgccctcctgcaatgctccctgtggaggccagtacacgggatcagaaggggtagttttatcaccaaactacccccataattacacagctggtcaaatatgcctctattccatcacggtaccaaaggaattcgtggtctttggacagtttgcctatttccagacagccctgaatgatttggcagaattatttgatggaacccatgcacaggccagacttctcagctcactctcggggtctcactcaggggaaacattgcccttggctacgtcaaatcaaattctgctccgattcagtgcaaagagcggtgcctctgcccgcggcttccacttcgtgtatcaagctgttcctcgtaccagtgacacccaatgcagctctgtccccgagcccagatacggaaggagaattggttctgagttttctgccggctccatcgtccgattcgagtgcaacccgggatacctgcttcagggttccacggcgctccactgccagtccgtgcccaacgccttggcacagtggaacgacacgatccccagctgtgtggtaccctgcagtggcaatttcactcaacgaagaggtacaatcctgtcccccggctaccctgagccatacggaaacaacttgaactgtatatggaagatcatagttacggagggctcgggaattcagatccaagtgatcagttttgccacggagcagaactgggactcccttgagatccacgatggtggggatgtgaccgcacccagactgggaagcttctcaggcaccacagtaccggcactgctgaacagtacttccaaccaactctacctgcatttccagtctgacattagtgtggcagctgctggtttccacctggaatacaaaactgtaggtcttgctgcatgccaagaaccagccctccccagcaacagcatcaaaatcggagatcggtacatggtgaacgacgtgctctccttccagtgcgagcccgggtacaccctgcagggccgttcccacatttcctgtatgccagggaccgttcgccgttggaactatccgtctcccctgtgcattgcaacctgtggagggacgctgagcaccttgggtggtgtgatcctgagccccggcttcccaggttcttaccccaacaacttagactgcacctggaggatctcattacccatcggctatggtgcacatattcagtttctgaatttttctaccgaagctaatcatgacttccttgaaattcaaaatggaccttaccacaccagccccatgattggacaatttagcggcacggatctccccgcggccctgctgagcacaacgcatgaaaccctcatccacttttatagtgaccattcgcaaaaccggcaaggatttaaacttgcttaccaagcctatgaattacagaactgtccagatccacccccatttcagaatgggtacatgatcaactcggattacagcgtggggcaatcagtatctttcgagtgttatcctgggtacattctaataggccatcctgtcctcacttgtcagcatgggatcaacagaaactggaactacccttttccaagatgtgatgccccttgtgggtacaacgtaacttctcagaacggcaccatctactcccctggctttcctgatgagtatccgatcctgaaggactgcatttggctcatcacggtgcctccagggcacggagtttacatcaacttcaccctgttacagacggaagctgtcaacgattacattgctgtttgggacggtcccgatcagaactcaccccagctgggagttttcagtggcaacacagccctcgaaacggcgtatagctccaccaaccaagtcctgctcaagttccacagcgacttttcaaatggaggcttctttgtcctcaatttccacgcatttcagctcaagaaatgtcaacctcccccagcggttccacaggcagaaatgcttactgaggatgatgatttcgaaataggagattttgtgaagtaccagtgccaccccgggtacaccttggtggggaccgacattctgacttgcaagctcagttcccagttgcagtttgagggttctctcccaacatgtgaagcacaatgcccagcaaatgaagtccggactggatcatcgggagtcattctcagtccagggtatccgggtaattattttaactcccagacttgctcttggagtattaaagtggaaccaaactacaacattaccatctttgtggacacatttcaaagtgaaaagcagtttgatgcactggaagtgtttgatggttcttctgggcaaagtcctctgctagtagtcttaagtgggaatcatactgaacaatcaaattttacaagcaggagtaatcagttatatctccgctggtccactgaccatgccaccagtaagaaaggattcaagattcgctatgcagcaccttactgcagtttgacccaccccctgaagaatgggggtattctaaacaggactgcaggagcggttggaagcaaagtgcattatttttgcaagcctggataccgaatggtcggccacagcaatgcaacctgtagacgaaacccacttggcatgtaccagtgggactccctcacgccactctgccaggctgtgtcctgtggaatcccagaatccccaggaaacggttcatttaccgggaacgagttcactttggacagtaaagtggtctatgaatgtcatgagggcttcaagcttgaatccagccagcaagcaacagccgtgtgtcaagaagatgggttgtggagtaacaaggggaagccgcccacgtgtaagccggtcgcttgccccagcattgaagctcagctctcagaacatgtcatctggaggctggtttcaggatccttgaatgagtacggtgctcaagtattgctgagctgcagtcctggttactacttagaaggctggaggctcctgcggtgccaggccaatgggacgtggaacataggagatgagaggccaagctgtcgagttatctcgtgtggaagcctttcctttcccccaaatggcaacaagattggaacgttgacagtttatggggccacagctatatttacgtgcaacaccggctacacgcttgtggggtctcatgtcagagagtgcttggcaaatgggctctggagcggcagcgaaactcgatgtctggctggccactgcggttccccagacccgattgtgaacggtcacattagtggagatggcttcagttacagagacacggtggtttaccagtgcaatcctggtttccggcttgtgggaacttccgtgaggatatgcctgcaagaccacaagtggtctggacaaacgcctgtctgtgtccccatcacatgtggtcaccctggaaaccctgcccacggattcactaatggcagtgagttcaacctgaatgatgtcgtgaatttcacctgcaacacgggctatttgctgcagggcgtgtctcgagcccagtgtcggagcaacggccagtggagtagccctctgcccacgtgtcgagtggtgaactgttctgatccaggctttgtggaaaatgccattcgtcacgggcaacagaacttccctgagagttttgagtatggaatgagtatcctgtaccattgcaagaagggattttacttgctgggatcttcagccttgacctgtatggcaaatggcttatgggaccgatccctgcccaagtgtttggctatatcgtgtggacacccaggggtccctgccaacgccgtcctcactggagagctgtttacctatggcgccgtcgtgcactactcctgcagagggagcgagagcctcataggcaacgacacgagagtgtgccaggaagacagtcactggagcggggcactgccccactgcacaggaaataatcctggattctgtggtgatccggggaccccagcacatgggtctcggcttggtgatgactttaagacaaagagtcttctccgcttctcctgtgaaatggggcaccagctgaggggctcccctgaacgcacgtgtttgctcaatgggtcatggtcaggactgcagccggtgtgtgaggccgtgtcctgtggcaaccctggcacacccaccaacggaatgattgtcagtagtgatggcattctgttctccagctcggtcatctatgcctgctgggaaggctacaagacctcagggctcatgacacggcattgcacagccaatgggacctggacaggcactgctcccgactgcacaattataagttgtggggatccaggcacactagcaaatggcatccagtttgggaccgacttcaccttcaacaagactgtgagctatcagtgtaacccaggctatgtcatggaagcagtcacatccgccactattcgctgtaccaaagacggcaggtggaatccgagcaaacctgtctgcaaagccgtgctgtgtcctcagccgccgccggtgcagaatggaacagtggagggaagtgatttccgctggggctccagcataagttacagctgcatggacggttaccagctctctcactccgccatcctctcctgtgaaggtcgcggggtgtggaaaggagagatcccccagtgtctccctgtgttctgcggagaccctggcatccccgcagaagggcgacttagtgggaaaagtttcacctataagtccgaagtcttcttccagtgcaaatctccatttatactcgtgggatcctccagaagagtctgccaagctgacggcacgtggagcggcatacaacccacctgcattgatcctgctcataacacctgcccagaccctggtacgccacactttggaatacagaatagctccagaggctatgaggttggaagcacggtttttttcaggtgcagaaaaggctaccatattcaaggttccacgactcgcacctgccttgccaatttaacatggagtgggatacagaccgaatgtatacctcatgcctgcagacagccagaaaccccggcacacgcggatgtgagagccatcgatcttcctactttcggctacaccttagtgtacacctgccatccaggctttttcctcgcagggggatctgagcacagaacatgtaaagcagacatgaaatggacaggaaagtcgcctgtgtgtaaaagtaaaggagtgagagaagttaatgaaacagttactaaaactccagttccttcagatgtctttttcgtcaattcactgtggaaggggtattatgaatatttagggaaaagacaacccgccactctaactgttgactggttcaatgcaacaagcagtaaggtgaatgccaccttcagcgaagcctcgccagtggagctgaagttgacaggcatttacaagaaggaggaggcccacttactcctgaaagcttttcaaattaaaggccaggcagatatttttgtaagcaagttcgaaaatgacaactggggactagatggttatgtgtcatctggacttgaaagaggaggatttacttttcaaggtgacattcatggaaaagactttggaaaatttaagctagaaaggcaagatcctttaaacccagatcaagactcttccagtcattaccacggcaccagcagtggctctgtggcggctgccattctggttcctttctttgctctaattttatcagggtttgcattttacctctacaaacacagaacgagaccaaaagttcaatacaatggctatgctgggcatgaaaacagcaatggacaagcatcgtttgaaaaccccatgtatgatacaaacttaaaacccacagaagccaaggctgtgaggtttgacacaactctgaacacagtctgtacagtggtatagccctcagtgccccaacaggactgattcatagccatacctctgatggacaagcagtgattcctttggtgccatataccactctcccttccactctggctttactgcagcgatcttcaaccttgtctactggcataagtgcagcggggatctctactcaaatgtgtcagggtcttctacggatcaaactacacatgcgttttcattccaaaagtgggttctaaatgcctggctgcatctgtatgaaatcaaggcacactccaggaagactgccacgtcgcgccaacacgtcatactcaatgcctcagactttcatatttctgtgttgctgagatgcctttcaatgcaatcgtctgggctcgtggatatgtccctcaggtgcggtgacagaatggtggcaccacgatatgtgttctcttgtgttgtttttcctttttaaacccccatgaacacgaatactctgaaaaaaataaaaagctttctggaagaagacacctttctgatagaggctcacacctacaaatgcttcactctgtccttccgagacctgacaagctttgaggacctcacagctcccctgtgtgttcatctctagggatgtttgcaatttcccagtcagctgttctgtcgcagaatgtttaatgcacaattttttgcactagtgtgttatgaatgactaagattctgataaaaaaaataaattatttacacagggtttatacacactatccattgtatataagcattatttcatattatcaagctaaacattcccccatcagcttagttggagtgttagggaaaagtattcctagatatggcacagattttaaaaggaaatacagtattgaagagatttattttattattgcttcaattagctccatttacgtgttgaattcattgaagaggtccaatgagaaaaaaacagaagcctccttatttcacacgttttcctcctttagtaccatcctcatccaattactgtctctctgatactacttaatagcagggggtttgcagaaatttctgtttgccatgtaaaactgtgaatagtaatttattttagatagtcgatgaacttgtgggttttagctcacaatgcagccttcccttttgcagtgttttttttttgtttttttttttttttgtcttttactgtgccatcgatctttgatattgcattgaaagacaatataccacagtagcaccttgaactcagtgaaaattgttcaggatcaaaataccaagtgttcttttagagggaaggaaaaagtacacacactctcctctcacaatgatatattttatacattcatttgttatttgtttcatgctttatgattccagatggaaaggtaatttcagtgacttttcaagtttaaattccattataggtaaatgataagttatgatgcaaataaaatctataagatccccagggcaaataaaaatcaaaacatgaagtagaagatgtggccgtgaggtagtttatgtaacaaattcaaagtgaaaatcatgtttacttttacttatacttatttgataaaaatatttttgaaacgatagtacttattttattatttgatatttcagttcctattcaattgtggcagattttctctgtttcacattttagattggcgttggtaatagaaatgtcagaatgttcaaattggccttcacgttgtcggagtgaacacattgacacctagctttaagactgatttatctgttggtgtactgaaggtttccatgtaggacttcaaatgtggaaaaggaaaagcagtcaggaaaatggggcattctttggagagtcacgcgttttgattcggacatttccgtagagctcggctcccagtgttgtgttcctcggtcgaaagggtctctgctgtttggggactcactggcctctcctagggactcctttgtcttgtgaaccccacgctgttggattctgtatcattatgctgaattctctgcacagttttccctggccaacctgcccacatccttggagatttgctttgccagtgggaatccttacattgctgtttcacagtagacgggacgaggtcagcgggagtcgtgctcctaacacacacattgaacgaaacagaagatgattgaaagtgtgaggaggctcgtgtgcaagggagaacagggttactatacatattagtgtatatatatacatacatatatatatatatatattgtacatatctaagtttgagtcattcaaactaggtgcaaaatgctgacttcagagtctgaattaacatctctgttcccatatccctgacctgctccctggtcaacgatgctatgaaatcctgaaatgacaggacatacatacatacaagaaaccacatatcaaattagatatgattttcctttgtgtgcaaagtcaaactgtcctagggttgccagtttgaagcatgttatttaaatgaaaaaaaaaatcagtgaaattctcgtgtgagaattctgcctagtttcttcctaaggttgtgtgcagtgttgaacggcgtctccgcaaggtgttggaggatctcattttagggcagtcaggagctgtgcttgctgagttaggtctagaagactcttccctgaaggcaacgggaacacgcgtgagggacgcgaccacacactaacagaggacacgtgcttcagagctgtttaaaactgctgcttgttttacacacacatcttgccttttttcaggctagctgcaataatttttttcttctgtaaaatattttgtaaacaacaacaaaaagctattataaaaagggggtaaaaaaaagaacgctggcattatgatcaggaaaacccattgtcatcgccgaccctccctcccgtcccaccacacgctgctgtcacgacgtaggtgcgaaagacctttttgtacagagatatattttttatgaagaatttgtaaaattattaaatatgctgtaattttttgattaatgtaggtacattgttaaaaaataaatgtttttacaatacagaactgtaattttcccaataatgtaaaatgtaccatctctagctgattttcagttccaatcctattacacatgtattaatattaaagtggcctgttaaaatgaacagtatcttttttttgtcaaaaaaattataaagagggtgtaatatagcctgtgcaatgccaccaatctttaaagcaaatcagagttctaattaaatatttaattttagatttctaaaaaaaaaaaaaaaaaaaaaaaa

SEQ ID NO: 10 - protein, 3564 aa, Homo sapiens
MTAWRRFQSLLLLLGLLVLCARLLTAAKGQNCGGLVQGPNGTIESPGFPHGYPNYANCTWIIITGERNRIQLSFHTFALEEDFDILSVYDGQPQQGNLKVRLSGFQLPSSIVSTGSILTLWFTTDFAVSAQGFKALYEVLPSHTCGNPGEILKGVLHGTRFNIGDKIRYSCLPGYILEGHAILTCIVSPGNGASWDFPAPFCRAEGACGGTLRGTSSSISSPHFPSEYENNADCTWTILAEPGDTIALVFTDFQLEEGYDFLEISGTEAPSIWLTGMNLPSPVISSKNWLRLHFTSDSNHRRKGFNAQFQVKKAIELKSRGVKMLPSKDGSHKNSVLSQGGVALVSDMCPDPGIPENGRRAGSDFRVGANVQFSCEDNYVLQGSKSITCQRVTETLAAWSDHRPICRARTCGSNLRGPSGVITSPNYPVQYEDNAHCVWVITTTDPDKVIKLAFEEFELERGYDTLTVGDAGKVGDTRSVLYVLTGSSVPDLIVSMSNQMWLHLQSDDSIGSPGFKAVYQEIEKGGCGDPGIPAYGKRTGSSFLHGDTLTFECPAAFELVGERVITCQQNNQWSGNKPSCVFSCFFNFTASSGIILSPNYPEEYGNNMNCVWLIISEPGSRIHLIFNDFDVEPQFDFLAVKDDGISDITVLGTFSGNEVPSQLASSGHIVRLEFQSDHSTTGRGFNITYTTFGQNECHDPGIPINGRRFGDRFLLGSSVSFHCDDGFVKTQGSESITCILQDGNVVWSSTVPRCEAPCGGHLTASSGVILPPGWPGYYKDSLHCEWIIEAKPGHSIKITFDRFQTEVNYDTLEVRDGPASSSPLIGEYHGTQAPQFLISTGNFMYLLFTTDNSRSSIGFLIHYESVTLESDSCLDPGIPVNGHRHGGDFGIRSTVTFSCDPGYTLSDDEPLVCERNHQWNHALPSCDALCGGYIQGKSGTVLSPGFPDFYPNSLNCTWTIEVSHGKGVQMIFHTFHLESSHDYLLITEDGSFSEPVARLTGSVLPHTIKAGLFGNFTAQLRFISDFSISYEGFNITFSEYDLEPCDDPGVPAFSRRIGFHFGVGDSLTFSCFLGYRLEGATKLTCLGGGRRVWSAPLPRCVAECGASVKGNEGTLLSPNFPSNYDNNHECIYKIETEAGKGIHLRTRSFQLFEGDTLKVYDGKDSSSRPLGTFTKNELLGLILNSTSNHLWLEFNTNGSDTDQGFQLTYTSFDLVKCEDPGIPNYGYRIRDEGHFTDTVVLYSCNPGYAMHGSNTLTCLSGDRRVWDKPLPSCIAECGGQIHAATSGRILSPGYPAPYDNNLHCTWIIEADPGKTISLHFIVFDTEMAHDILKVWDGPVDSDILLKEWSGSALPEDIHSTFNSLTLQFDSDFFISKSGFSIQFSTSIAATCNDPGMPQNGTRYGDSREAGDTVTFQCDPGYQLQGQAKITCVQLNNRFFWQPDPPTCIAACGGNLTGPAGVILSPNYPQPYPPGKECDWRVKVNPDFVIALIFKSFNMEPSYDFLHIYEGEDSNSPLIGSYQGSQAPERIESSGNSLFLAFRSDASVGLSGFAIEFKEKPREACFDPGNIMNGTRVGTDFKLGSTITYQCDSGYKILDPSSITCVIGADGKPSWDQVLPSCNAPCGGQYTGSEGVVLSPNYPHNYTAGQICLYSITVPKEFVVFGQFAYFQTALNDLAELFDGTHAQARLLSSLSGSHSGETLPLATSNQILLRFSAKSGASARGFHFVYQAVPRTSDTQCSSVPEPRYGRRIGSEFSAGSIVRFECNPGYLLQGSTALHCQSVPNALAQWNDTIPSCVVPCSGNFTQRRGTILSPGYPEPYGNNLNCIWKIIVTEGSGIQIQVISFATEQNWDSLEIHDGGDVTAPRLGSFSGTTVPALLNSTSNQLYLHFQSDISVAAAGFHLEYKTVGLAACQEPALPSNSIKIGDRYMVNDVLSFQCEPGYTLQGRSHISCMPGTVRRWNYPSPLCIATCGGTLSTLGGVILSPGFPGSYPNNLDCTWRISLPIGYGAHIQFLNFSTEANHDFLEIQNGPYHTSPMIGQFSGTDLPAALLSTTHETLIHFYSDHSQNRQGFKLAYQAYELQNCPDPPPFQNGYMINSDYSVGQSVSFECYPGYILIGHPVLTCQHGINRNWNYPFPRCDAPCGYNVTSQNGTIYSPGFPDEYPILKDCIWLITVPPGHGVYINFTLLQTEAVNDYIAVWDGPDQNSPQLGVFSGNTALETAYSSTNQVLLKFHSDFSNGGFFVLNFHAFQLKKCQPPPAVPQAEMLTEDDDFEIGDFVKYQCHPGYTLVGTDILTCKLSSQLQFEGSLPTCEAQCPANEVRTGSSGVILSPGYPGNYFNSQTCSWSIKVEPNYNITIFVDTFQSEKQFDALEVFDGSSGQSPLLVVLSGNHTEQSNFTSRSNQLYLRWSTDHATSKKGFKIRYAAPYCSLTHPLKNGGILNRTAGAVGSKVHYFCKPGYRMVGHSNATCRRNPLGMYQWDSLTPLCQAVSCGIPESPGNGSFTGNEFTLDSKVVYECHEGFKLESSQQATAVCQEDGLWSNKGKPPTCKPVACPSIEAQLSEHVIWRLVSGSLNEYGAQVLLSCSPGYYLEGWRLLRCQANGTWNIGDERPSCRVISCGSLSFPPNGNKIGTLTVYGATAIFTCNTGYTLVGSHVRECLANGLWSGSETRCLAGHCGSPDPIVNGHISGDGFSYRDTVVYQCNPGFRLVGTSVRICLQDHKWSGQTPVCVPITCGHPGNPAHGFTNGSEFNLNDVVNFTCNTGYLLQGVSRAQCRSNGQWSSPLPTCRVVNCSDPGFVENAIRHGQQNFPESFEYGMSILYHCKKGFYLLGSSALTCMANGLWDRSLPKCLAISCGHPGVPANAVLTGELFTYGAVVHYSCRGSESLIGNDTRVCQEDSHWSGALPHCTGNNPGFCGDPGTPAHGSRLGDDFKTKSLLRFSCEMGHQLRGSPERTCLLNGSWSGLQPVCEAVSCGNPGTPTNGMIVSSDGILFSSSVIYACWEGYKTSGLMTRHCTANGTWTGTAPDCTIISCGDPGTLANGIQFGTDFTFNKTVSYQCNPGYVMEAVTSATIRCTKDGRWNPSKPVCKAVLCPQPPPVQNGTVEGSDFRWGSSISYSCMDGYQLSHSAILSCEGRGVWKGEIPQCLPVFCGDPGIPAEGRLSGKSFTYKSEVFFQCKSPFILVGSSRRVCQADGTWSGIQPTCIDPAHNTCPDPGTPHFGIQNSSRGYEVGSTVFFRCRKGYHIQGSTTRTCLANLTWSGIQTECIPHACRQPETPAHADVRAIDLPTFGYTLVYTCHPGFFLAGGSEHRTCKADMKWTGKSPVCKSKGVREVNETVTKTPVPSDVFFVNSLWKGYYEYLGKRQPATLTVDWFNATSSKVNATFSEASPVELKLTGIYKKEEAHLLLKAFQIKGQADIFVSKFENDNWGLDGYVSSGLERGGFTFQGDIHGKDFGKFKLERQDPLNPDQDSSSHYHGTSSGSVAAAILVPFFALILSGFAFYLYKHRTRPKVQYNGYAGHENSNGQASFENPMYDTNLKPTEAKAVRFDTTLNTVCTVV

SEQ ID NO: 11 - DNA, 12351 bp, Homo sapiens
atgggagctacagggcgcctcgagctcacactggccgcccctccccatccgggcccagcctttcagcgttcaaaagccagggagacccaaggagaggaggaagggagtgaaatgcagatcgccaaaagtgactccatacatcacatgagccactcccaggggcagccagagctgcctcctctgcctgcttctgctaatgaggaaccgtctggactctatcagactgtcatgtcacacagcttttacccgcccttgatgcaacgcacgtcatggaccttggctgcacccttcaaagaacagcatcaccaccgtggacccagtgattccatcgccaacaactactccttgatggcccaggacctgaagctgaaagatctgctgaaggtctaccaaccggccaccatcagtgtccctagggacaggaccggtcaggggctgccatcatcaggaaatagaagctcatcagagcccatgaggaaaaaaacgaagttttcctccagaaacaaagaggattccactaggatcaagttggccttcaagacgtcaatcttctcacccatgaagaaggaggtaaagacatctttgacgttcccaggaagcagaccaatgagtccagaacagcagctcgatgtcatgttacagcaggagatggaaatggaaagtaaagaaaagaagccatctgaatcggacctggagagatactattactatctgaccaatggaattcgcaaagacatgattgcccctgaggagggtgaagtgatggttcggatttcaaagctgatttctaacacgctgctgacgagtcccttcctggagcccctgatggtggtcctcgtgcaggagaaggagaatgactattactgtagcctcatgaaaagcatcgttgattacatcctcatggacccaatggagagaaaacggctctttattgagagcatcccccgcttgtttcctcaaagagtgatccgggcccctgtgccctggcacagtgtctacaggagcgccaagaagtggaacgaggagcatctgcacacggtgaaccccatgatgctcaggctgaaagaactgtggtttgcagaattcagagacctcaggtttgttcgaacagcagaaatactagcgggaaaattgcctctgcagcctcaggaattttgggatgtgatccagaaacactgcctggaggcacaccagactcttctcaacaagtggatccccacctgcgcccagctttttacctcacggaaggagcactggattcattttgctcccaagagcaactatgactcaagtcgaaacattgaggaatattttgcttctgtggcatcattcatgtcgctgcagcttagggagctggtcattaagtcacttgaggacctcgtttcccttttcatgatacacaaagatgggaatgattttaaggagccctaccaagagatgaagtttttcatacctcagctaatcatgatcaaacttgaagtcagtgaacccattattgtcttcaatccatcttttgatggctgctgggaattaatacgtgactctttcctggaaattattaagaactctaatgggatccccaagctgaaatacataccacttaagttctccttcactgctgctgctgctgatcggcaatgtgtgaaagcagctgagccaggagagcccagcatgcacgcggctgccactgcaatggcagagctgaaaggatataatctgctccttggaactgttaacgcagaagaaaaacttgtttctgattttttgattcaaactttcaaggtatttcagaaaaatcaagttggcccctgcaaatatttaaatgtctacaaaaagtatgttgacttattggataacacggcagagcaaaacatcgctgcgttcctgaaagaaaatcatgacattgatgattttgtgacgaagatcaatgccataaagaaacggagaaatgaaattgcatccatgaacatcaccgtgcctttagccatgttctgccttgatgctacggccctaaatcatgatctctgtgagcgagctcaaaatcttaaagaccatctgattcaattccaagtggatgtaaaccgagacaccaataccagcatttgtaatcagtacagccacatcgcagacaaagtcagtgaggttcctgccaacactaaggagctggtatccctcattgaattcctaaagaaatccagtgctgtcactgtgttcaaactcaggaggcaacttagagatgcaagtgaacggctggagttcctgatggactatgcagacttgccgtaccagattgaagatatctttgacaacagccggaacttgctccttcacaagagggatcaggcagaaatggatctgattaaaagatgctcagaatttgagttgagacttgagggctaccacagagaactggaaagttttaggaagcgcgaagtgatgactacagaagaaatgaagcacaatgttgaaaagcttaatgagctttcaaagaacctaaatcgggcgtttgcagagtttgagttgatcaataaggaggaagagctattggaaaaggagaagagtacttaccctcttctgcaggccatgctgaagaacaaagtaccttatgagcagctgtggtcgacagcctatgagttcagcatcaagtcagaggaatggatgaatggacccctcttcttactgaatgctgagcaaattgcggaggagatagggaatatgtggaggacaacgtataaactgatcaagaccttgtctgatgtgcctgcacccaggcgcttagcagagaatgtgaagatcaagatcgataagttcaagcagtacattcccatcctcagtatttcctgcaacccaggaatgaaagaccgacactggcagcagatcagtgagattgttggctatgagataaagcccaccgaaacgacctgcctctcaaatatgctcgaatttggattcggcaaattcgttgaaaaattggagcccattggtgcagctgccagcaaggaatactctctggagaaaaacttggatagaatgaagttggattgggttaacgtgacgttcagcttcgtgaaatacagggacactgatacaaacatcttgtgtgcaattgatgacattcaaatgctacttgatgatcacgtgataaagacccagaccatgtgtggctccccattcatcaaaccaatagaagcagaatgccggaaatgggaagaaaagctaattcgcatacaagacaatttggatgcctggttgaaatgccaagccacctggctgtacctggaaccaatcttcagttcagaggacatcatagcccagatgccagaagaggggaggaaatttggcattgttgatagttactggaaatcacttatgtcccaagcggtgaaagataacaggattctggtggcagccgaccagccacggatggcagagaagcttcaagaagccaactttctcttggaggacatccagaaagggctgaatgattacttggagaagaagagactattcttccccagattcttcttcctatcaaacgatgagctgctggaaatcttgtccgagacaaaggaccctctccgagtgcagccgcacttgaagaagtgctttgaaggaattgccaagcttgagtttacagacaatctggaaattgtgggcatgatcagctcggaaaaagaaactgttccattcatacagaaaatctacccagctaatgccaagggcatggtggaaaagtggctccagcaggtggagcagatgatgctggccagtatgcgagaagtcattggacttgggattgaagcatatgtcaaggtccctcgaaatcactgggtcttacagtggcctggacaggtggttatctgtgtctcctccatcttttggacccaggaggtgtcccaagccctggcggaaaataccttactggattttctgaaaaagagcaatgatcagattgcgcagattgtccagctggtgcgagggaagctgagcagtggagctcgactcactctcggggccctcacggtcatcgatgtccacgcccgcgacgtggtggccaagttatctgaggacagggtctccgatctgaatgatttccaatggatctcacagctgcgctactactgggtggccaaggatgtgcaggtgcagattatcaccacagaagccttgtatggctatgagtacctgggaaactccccccggctggtgatcacacccctcaccgaccgctgctacaggacactgatgggagctttgaagctgaaccttgggggtgctccagagggtccagctgggactggcaagacagaaaccaccaaagatttggccaaagccttggctaagcagtgtgtggtcttcaactgctccgatggtttggattacaaagctatggggaagttcttcaaggggctggcacaggctggagcatgggcgtgctttgatgagttcaacaggatcgaggtagaagtgctgtctgtggtcgctcagcagatcctcagcatccaacaagccatcattcggaagctaaagacattcatctttgaagggactgagctctctctgaacccaacctgcgctgtgttcatcaccatgaaccccgggtatgctggcagggctgaactgcccgacaatctcaaggccttgttccggacagtggccatgatggtcccagattacgccctcattggagaaatctccctctactccatggggtttctggactccagaagtctcgcccagaagatcgttgcgacctaccgcctgtgctcggaacaactgtcctctcagcatcactatgactacggtatgcgcgctgtcaagtctgtgcttactgccgcaggaaacctgaagctcaagtatccagaggagaatgaaagtgtcctgctgctccgggcattgcttgatgtcaatctggccaagttcttagcgcaagatgtccctctgtttcagggaattatatctgatttatttcctggagttgttcttccaaagccagactatgaagtttttctgaaagtgctgaatgataacatcaaaaagatgaaactccagccagtaccttggtttatagggaaaattatccagatctacgaaatgatgctggtgagacatggctatatgattgtaggagaccccatgggcggcaagacctctgcttataaagtgttggctgcagctctcggcgatttacacgcagccaatcagatggaggagtttgctgtggagtacaagatcatcaaccccaaggctatcacgatggggcagctgtatgggtgctttgaccaagtgagccacgagtggatggatggtgtccttgccaatgctttccgggagcaagcgtcttcactctctgatgatcgcaagtggattatatttgatgggccagtggatgctatttggattgaaaatatgaacactgttctggatgacaataaaaagctgtgtctcatgagtggggaaattatccagatgaactccaagatgagcctgatcttcgagcccgccgacctcgagcaagcctctccagccactgtgagcaggtgtgggatgatctacatggagccccatcaactaggctggaagcccctgaaggattcctacatggacaccctgccctccagtctcaccaaggagcacaaagaattggtcaatgacatgttcatgtggcttgtccagccctgcctggaatttggtcgccttcattgtaaatttgttgtccagacatctcccatccaccttgccttctcaatgatgagactgtactcttctctgcttgatgaaatcagggcagtagaagaggaggaaatggaattaggtgaaggcctgtcaagtcaacagatctttctctggctccaaggactgtttctcttttccttggtgtggaccgtggctggcaccatcaacgcagacagcagaaagaaatttgatgtgtttttccgcaacctgatcatgggcatggatgataaccacccaaggcccaaaagcgtcaaactcaccaaaaacaacatctttccagaaagaggaagcatctatgatttttattttatcaaacaagctagtggacattgggaaacgtggacacagtatatcaccaaagaggaggaaaaagttccagctggtgcaaaggtctcagaactcatcatccccacaatggagacagcccggcagtccttcttcttgaaaacctacttagaccatgagattccaatgctgttcgtgggtcccacaggcactggcaaatcagccatcaccaacaacttccttctccaccttcccaaaaatacgtacctacccaactgcatcaatttctctgccagaacctcagccaatcagacccaggatatcatcatgtccaagctggatcgacgacggaagggccttttcgggcctcccatagggaagaaagcagtggtgtttgtggatgacctcaacatgccagccaaagaggtgtatggggcccagccacccatcgagctcctgaggcagtggattgaccatggttactggtttgacaagaaagacacaaccaggctggacatcgtggacatgctgctcgtgacagccatggggccccccgggggaggaaggaatgacattactggacgattcactcgccatctgaatatcatttccatcaatgcctttgaggatgacattttaaccaagattttcagttcgattgttgactggcacttcgggaaagggtttgatgtgatgtttttaaggtacggaaagatgctggtccaagctactaagacaatttatagagatgcagtggagaacttcttgccaactccctcgaagtcacattacgtctttaacctgcgggacttctcacgagtgattcaaggggtcctgctgtgccctcacacacacctgcaggatgtagaaaaatgtatccggctttggatccatgaggtttatcgggtcttctatgatcgtctgattgacaaggaggacagacaggtctttttcaacatggtgaaggaaaccacctccaattgcttcaagcagaccatagagaaggtgcttatccacttgtcacccactggaaagatagtcgatgataacattcgaagcctcttctttggagattatttcaagccagaaagtgaccaaaaaatctacgatgagatcactgacctgaaacagctgactgtggtcatggagcactatctggaagaattcaacaacatcagcaaggcccccatgtccctggtcatgttcaggtttgccattgagcacatctctaggatctgccgtgtcctgaagcaggacaaaggccacctgctcctggtgggcatagggggcagcgggcggcaaagtgccgccaaactgtccacattcatgaacgcatacgagctataccagattgagatcaccaagaactacgcaggcaatgactggcgagaagatcttaagaagatcatactgcaggtcggtgtggccaccaagagcaccgtgttcctcttcgccgacaaccagatcaaggatgaatcattcgtggaggacatcaacatgcttctgaacacaggtgacgtgcctaacatcttccctgctgacgagaaggctgacatcgtggagaagatgcagactgcagccaggacccaaggagagaaggttgaagtcactcctctttctatgtataacttctttattgagagggtaattaacaaaatctccttttcattagccatgagtccaataggggatgccttcaggaaccgcctgcggatgttcccttcgctgatcaattgctgtacgattgattggttccagtcctggcccacagatgccctagagttggtggctaacaaatttctagaggatgtggagcttgatgacaacattcgggtagaggtcgtgtccatgtgcaaatatttccaagagagcgtcaagaagctgtcactcgattattacaacaaacttcgaagacacaactatgttacccccacctcctaccttgaattgattctaaccttcaagacgctcctgaatagcaagaggcaagaggtggctatgatgaggaaccgctacctgacaggcttgcagaaactcgactttgcagcttctcaggtagcggttatgcaaagagaactgacagctcttcaacctcaactcatcctcacctccgaggaaactgccaagatgatggtgaaaattgaagcggagacgagagaagctgatggaaagaaacttctggtgcaggcagatgaaaaagaagccaatgttgctgctgccattgcccaaggaatcaagaacgaatgtgagggggacctagctgaggcaatgcctgcactcgaggctgcactagctgctctggacaccctgaacccggccgacatctcgctggtgaagtcgatgcagaacccaccaggccctgtcaaactggtcatggagagcatctgcatcatgaaagggatgaagccagagaggaagccagaccccagtggctccggtaagatgatagaagattactggggggtatccaaaaagattcttggggatctgaaattcttggagagtcttaagacatatgacaaagacaacatccccccactgaccatgaagcggatccgggaaaggtttatcaatcacccggaattccagccagctgtcattaaaaatgtatcgtcggcctgcgagggtctgtgcaagtgggtgagggccatggaggtgtacgatcgcgtggccaaggtggtggctcccaaacgggagcgactgagggaggcagaggggaagctggctgcacagatgcagaagctgaaccagaaaagagcagagctgaagctggtggtagatcggctccaggccctgaatgacgactttgaagagatgaacaccaagaaaaaggacttggaggaaaacattgaaatctgctcccaaaagctggtcagggcagagaaactgatcagtggtcttgggggagagaaggacagatggaccgaagctgcccgacagctggggatccgctatactaatctgactggtgacgtgttgctgtcctcaggaactgtggcttacctgggcgcttttacagtggattatcgggtccagtgccaaaatcagtggttggctgaatgtaaggacaaggtcatccctggcttcagtgacttcagtctcagccacacgttaggggatcccataaaaatccgtgcctggcagattgctgggcttcccgttgactccttctccatcgacaatggcatcattgtatccaattccagacgctgggccttaatgattgaccctcacgggcaggccaataaatggattaagaacatggagaaggcgaataaactggctgtcatcaagttctctgatagcaactacatgaggatgctggaaaacgcgctgcagttaggcacccctgtcttgattgaaaacattggagaagagctggatgcttctatcgaacctatcttgctcaaggcaacattcaaacagcaaggagttgagtacatgaggctgggtgaaaacatcattgaatattccagggattttaagttatacatcacaacccgtttgaggaatccacattacctcccagaagttgccgtgaaggtctgtctcctcaacttcatgatcacccccttgggtctccaagatcaactccttggcatcgtggctgcgaaggagaagccagagctggaagagaaaaagaaccagttgattgtggaaagtgccaagaacaagaagcatctcaaggaaattgaagataagatcttggaggttctctccatgtccaagggtaacatcctggaggatgaaaccgccatcaaagttctgtcctcctccaaagtgctatctgaagagatctcagagaaacagaaagttgcttccatgacagaaacgcagattgacgagactcggatgggctacaagccagtggctgtgcattctgccaccatcttcttttgtatctcggacctggccaacatcgagccgatgtaccagtactccctgacttggttcataaatctctacatgcattccttgacccacagcacgaagagcgaggaactgaatctgcgcatcaagtacatcattgaccatttcaccctgagcatctacaacaacgtgtgccgttctctgtttgagaaggacaagctactcttctctctcctcctgaccatcggcatcatgaaacagaagaaggaaattacggaggaggtgtggtacttccttctcactggaggcatcgcactggataacccctaccccaatccagctccccaatggctgtctgagaaggcatgggcagagattgtccgtgcatctgccttacccaaactgcatggcctgatggagcatttggaacagaacctgggtgaatggaagctgatctatgactcggcctggccccatgaggagcaactccctgggtcttggaagttctctcaaggattggagaagatggtgatccttcgatgtttgcggcctgacaaaatggtgccagcggtccgggagttcattgctgaacatatgggaaagctgtatatcgaagcccctacgttcgatctccagggatcctacaatgattccagctgctgtgcgcctttgatttttgtgttgtctccaagtgcagacccaatggcaggcctgctgaagtttgctgatgatcttggtatgggaggtaccagaacacagaccatctcccttggccaaggccaaggccctattgctgccaaaatgatcaacaatgccatcaaagacgggacctgggtggtcttacagaactgccacctggccgcaagctggatgcctaccctggagaagatttgtgaggaggtgattgttcctgagagcaccaatgccagattcagactctggctaaccagctatccatcagagaagtttccagtcagcattctccagaatggaatcaaaatgaccaatgagccccccaaagggctccgggccaacctgttgcgctcctacctcaatgaccccatctcagatcctgtgttcttccaaagctgtgcaaaggcggtgatgtggcaaaagatgttatttggcctttgtttcttccacgccgttgttcaagagagaagaaacttcggccccctagggtggaatattccctatgaattcaacgaatctgacctgaggattagtatgtggcagatccagatgtttctcaatgactacaaggaggtgccctttgatgctctgacctacctgacaggggaatgtaattacggaggcagagtgactgatgacaaagaccggcgtctcctgctgtcacttctgtccatgttctactgtaaggaaattgaggaggactattactccctcgctcctggagacacttactacatccctcctcatggctcctaccagtcctatatcgactatctcaggaatctccccatcacagcccacccagaagtgttcggcctccatgagaacgcagacatcaccaaagacaaccaggaaaccaaccagctgtttgagggggtcctgctgaccctccctagacagtcaggaggaagtggcaagtcccctcaggaagtggttgaggagttggcacaagacattctctccaagcttcccagagactttgacctggaagaggtcatgaagttgtaccccgtggtctatgaagaatccatgaataccgtcctaaggcaggagctcatcagattcaacaggctgaccaaagtggttcggaggagcctcatcaatcttggccgagccatcaaaggacaggtcctgatgtcctcggagctagaggaagtctttaacagcatgcttgtgggtaaagtgccagccatgtgggcagccaagtcttacccatcactgaagcctctggggggctacgtggctgacctgctggcccgcctgaccttcttccaggaatggattgacaaggggccccctgtggtattttggatctctggattctacttcacacagtcttttttgactggcgtctctcaaaattatgcccggaaatataccatccccattgaccacattggatttgagtttgaggtaaccccacaagaaacagtgatggagaataaccccgaagatggggcctacatcaaagggctcttcttagaaggtgcccgttgggacaggaaaacgatgcagattggggaatctctccccaaaatcctctatgacccactgcccatcatttggctgaaacctggggagagcgcaatgtttctgcatcaggacatctatgtgtgtccagtctacaaaacaagtgcccgcagaggaaccctctccaccacaggccactctaccaactatgtcctctccattgagcttccaacagacatgccccagaagcactggataaaccgaggggtggcctcactgtgccagctggataactga

SEQ ID NO: 12 - protein, 4116 aa, Homo sapiens
MGATGRLELTLAAPPHPGPAFQRSKARETQGEEEGSEMQIAKSDSIHHMSHSQGQPELPPLPASANEEPSGLYQTVMSHSFYPPLMQRTSWTLAAPFKEQHHHRGPSDSIANNYSLMAQDLKLKDLLKVYQPATISVPRDRTGQGLPSSGNRSSSEPMRKKTKFSSRNKEDSTRIKLAFKTSIFSPMKKEVKTSLTFPGSRPMSPEQQLDVMLQQEMEMESKEKKPSESDLERYYYYLTNGIRKDMIAPEEGEVMVRISKLISNTLLTSPFLEPLMVVLVQEKENDYYCSLMKSIVDYILMDPMERKRLFIESIPRLFPQRVIRAPVPWHSVYRSAKKWNEEHLHTVNPMMLRLKELWFAEFRDLRFVRTAEILAGKLPLQPQEFWDVIQKHCLEAHQTLLNKWIPTCAQLFTSRKEHWIHFAPKSNYDSSRNIEEYFASVASFMSLQLRELVIKSLEDLVSLFMIHKDGNDFKEPYQEMKFFIPQLIMIKLEVSEPIIVFNPSFDGCWELIRDSFLEIIKNSNGIPKLKYIPLKFSFTAAAADRQCVKAAEPGEPSMHAAATAMAELKGYNLLLGTVNAEEKLVSDFLIQTFKVFQKNQVGPCKYLNVYKKYVDLLDNTAEQNIAAFLKENHDIDDFVTKINAIKKRRNEIASMNITVPLAMFCLDATALNHDLCERAQNLKDHLIQFQVDVNRDTNTSICNQYSHIADKVSEVPANTKELVSLIEFLKKSSAVTVFKLRRQLRDASERLEFLMDYADLPYQIEDIFDNSRNLLLHKRDQAEMDLIKRCSEFELRLEGYHRELESFRKREVMTTEEMKHNVEKLNELSKNLNRAFAEFELINKEEELLEKEKSTYPLLQAMLKNKVPYEQLWSTAYEFSIKSEEWMNGPLFLLNAEQIAEEIGNMWRTTYKLIKTLSDVPAPRRLAENVKIKIDKFKQYIPILSISCNPGMKDRHWQQISEIVGYEIKPTETTCLSNMLEFGFGKFVEKLEPIGAAASKEYSLEKNLDRMKLDWVNVTFSFVKYRDTDTNILCAIDDIQMLLDDHVIKTQTMCGSPFIKPIEAECRKWEEKLIRIQDNLDAWLKCQATWLYLEPIFSSEDIIAQMPEEGRKFGIVDSYWKSLMSQAVKDNRILVAADQPRMAEKLQEANFLLEDIQKGLNDYLEKKRLFFPRFFFLSNDELLEILSETKDPLRVQPHLKKCFEGIAKLEFTDNLEIVGMISSEKETVPFIQKIYPANAKGMVEKWLQQVEQMMLASMREVIGLGIEAYVKVPRNHWVLQWPGQVVICVSSIFWTQEVSQALAENTLLDFLKKSNDQIAQIVQLVRGKLSSGARLTLGALTVIDVHARDVVAKLSEDRVSDLNDFQWISQLRYYWVAKDVQVQIITTEALYGYEYLGNSPRLVITPLTDRCYRTLMGALKLNLGGAPEGPAGTGKTETTKDLAKALAKQCVVFNCSDGLDYKAMGKFFKGLAQAGAWACFDEFNRIEVEVLSVVAQQILSIQQAIIRKLKTFIFEGTELSLNPTCAVFITMNPGYAGRAELPDNLKALFRTVAMMVPDYALIGEISLYSMGFLDSRSLAQKIVATYRLCSEQLSSQHHYDYGMRAVKSVLTAAGNLKLKYPEENESVLLLRALLDVNLAKFLAQDVPLFQGIISDLFPGVVLPKPDYEVFLKVLNDNIKKMKLQPVPWFIGKIIQIYEMMLVRHGYMIVGDPMGGKTSAYKVLAAALGDLHAANQMEEFAVEYKIINPKAITMGQLYGCFDQVSHEWMDGVLANAFREQASSLSDDRKWIIFDGPVDAIWIENMNTVLDDNKKLCLMSGEIIQMNSKMSLIFEPADLEQASPATVSRCGMIYMEPHQLGWKPLKDSYMDTLPSSLTKEHKELVNDMFMWLVQPCLEFGRLHCKFVVQTSPIHLAFSMMRLYSSLLDEIRAVEEEEMELGEGLSSQQIFLWLQGLFLFSLVWTVAGTINADSRKKFDVFFRNLIMGMDDNHPRPKSVKLTKNNIFPERGSIYDFYFIKQASGHWETWTQYITKEEEKVPAGAKVSELIIPTMETARQSFFLKTYLDHEIPMLFVGPTGTGKSAITNNFLLHLPKNTYLPNCINFSARTSANQTQDIIMSKLDRRRKGLFGPPIGKKAVVFVDDLNMPAKEVYGAQPPIELLRQWIDHGYWFDKKDTTRLDIVDMLLVTAMGPPGGGRNDITGRFTRHLNIISINAFEDDILTKIFSSIVDWHFGKGFDVMFLRYGKMLVQATKTIYRDAVENFLPTPSKSHYVFNLRDFSRVIQGVLLCPHTHLQDVEKCIRLWIHEVYRVFYDRLIDKEDRQVFFNMVKETTSNCFKQTIEKVLIHLSPTGKIVDDNIRSLFFGDYFKPESDQKIYDEITDLKQLTVVMEHYLEEFNNISKAPMSLVMFRFAIEHISRICRVLKQDKGHLLLVGIGGSGRQSAAKLSTFMNAYELYQIEITKNYAGNDWREDLKKIILQVGVATKSTVFLFADNQIKDESFVEDINMLLNTGDVPNIFPADEKADIVEKMQTAARTQGEKVEVTPLSMYNFFIERVINKISFSLAMSPIGDAFRNRLRMFPSLINCCTIDWFQSWPTDALELVANKFLEDVELDDNIRVEVVSMCKYFQESVKKLSLDYYNKLRRHNYVTPTSYLELILTFKTLLNSKRQEVAMMRNRYLTGLQKLDFAASQVAVMQRELTALQPQLILTSEETAKMMVKIEAETREADGKKLLVQADEKEANVAAAIAQGIKNECEGDLAEAMPALEAALAALDTLNPADISLVKSMQNPPGPVKLVMESICIMKGMKPERKPDPSGSGKMIEDYWGVSKKILGDLKFLESLKTYDKDNIPPLTMKRIRERFINHPEFQPAVIKNVSSACEGLCKWVRAMEVYDRVAKVVAPKRERLREAEGKLAAQMQKLNQKRAELKLVVDRLQALNDDFEEMNTKKKDLEENIEICSQKLVRAEKLISGLGGEKDRWTEAARQLGIRYTNLTGDVLLSSGTVAYLGAFTVDYRVQCQNQWLAECKDKVIPGFSDFSLSHTLGDPIKIRAWQIAGLPVDSFSIDNGIIVSNSRRWALMIDPHGQANKWIKNMEKANKLAVIKFSDSNYMRMLENALQLGTPVLIENIGEELDASIEPILLKATFKQQGVEYMRLGENIIEYSRDFKLYITTRLRNPHYLPEVAVKVCLLNFMITPLGLQDQLLGIVAAKEKPELEEKKNQLIVESAKNKKHLKEIEDKILEVLSMSKGNILEDETAIKVLSSSKVLSEEISEKQKVASMTETQIDETRMGYKPVAVHSATIFFCISDLANIEPMYQYSLTWFINLYMHSLTHSTKSEELNLRIKYIIDHFTLSIYNNVCRSLFEKDKLLFSLLLTIGIMKQKKEITEEVWYFLLTGGIALDNPYPNPAPQWLSEKAWAEIVRASALPKLHGLMEHLEQNLGEWKLIYDSAWPHEEQLPGSWKFSQGLEKMVILRCLRPDKMVPAVREFIAEHMGKLYIEAPTFDLQGSYNDSSCCAPLIFVLSPSADPMAGLLKFADDLGMGGTRTQTISLGQGQGPIAAKMINNAIKDGTWVVLQNCHLAASWMPTLEKICEEVIVPESTNARFRLWLTSYPSEKFPVSILQNGIKMTNEPPKGLRANLLRSYLNDPISDPVFFQSCAKAVMWQKMLFGLCFFHAVVQERRNFGPLGWNIPYEFNESDLRISMWQIQMFLNDYKEVPFDALTYLTGECNYGGRVTDDKDRRLLLSLLSMFYCKEIEEDYYSLAPGDTYYIPPHGSYQSYIDYLRNLPITAHPEVFGLHENADITKDNQETNQLFEGVLLTLPRQSGGSGKSPQEVVEELAQDILSKLPRDFDLEEVMKLYPVVYEESMNTVLRQELIRFNRLTKVVRRSLINLGRAIKGQVLMSSELEEVFNSMLVGKVPAMWAAKSYPSLKPLGGYVADLLARLTFFQEWIDKGPPVVFWISGFYFTQSFLTGVSQNYARKYTIPIDHIGFEFEVTPQETVMENNPEDGAYIKGLFLEGARWDRKTMQIGESLPKILYDPLPIIWLKPGESAMFLHQDIYVCPVYKTSARRGTLSTTGHSTNYVLSIELPTDMPQKHWINRGVASLCQLDN

SEQ ID NO: 13 - DNA, 2297 bp, Homo sapiens
aggatcagactttttaaatgtttggaattcaagatactttaggaagaggaccaactctgaaagagaaatcgctgggcgcggagatggattcggtcaggtcctgggtccggaatgtcggagtggtggacgctaatgtcgccgcgcagagcggggtcgccctgtcccgggcccactttgagaaacagcctccttccaacttgaggaaatccaacttctttcacttcgtcctggcgctctatgacaggcagggccagccggtggagatcgagcggacggccttcgtggactttgtggagaatgacaaagaacaaggcaacgagaagaccaacaacggcactcactacaagttacagctcctctacagcaacggtgtccgcacggaacaggacctctatgtcaggctcatcgactcggtcaccaagcagcccatcgcttacgagggacagaataagaatccggaaatgtgccgagttctcctgacgcacgaagtgatgtgtagtcgatgctgcgaaaagaaaagctgtggaaaccgaaatgagactccatcggacccagtcataattgacagattctttttaaaatttttcctcaagtgcaatcagaattgtttgaaaacagcaggaaacccaagggacatgagacggtttcaggttgtgttgtcaacaacggtgaatgtggatggacacgtcctggctgtttctgacaacatgtttgttcataacaactccaagcatggacggagagcaagaagactcgatccatcggaagctaccccctgcatcaaagccattagcccgagtgaaggctggaccacaggaggagccatggtcatcatcatcggggacaacttctttgatggtctccaagtggtgtttgggactatgcttgtatggagcgagctaataacccctcatgccatcagagtacagactcctccccggcacatcccaggcgtggtagaggtgacattatcttataaatctaaacagttctgcaaaggagccccaggaaggttcatttacacagcattaaatgaacccaccatagactatggcttccagagactgcagaaggtcatccctaggcatcctggagatcctgagagattagctaaggagatgctgttgaaaagagctgcagatctagtggaagctctttatggcacaccacacaataaccaggacatcattttgaagcgagccgcagacattgctgaagctctctacagcgtccccaggaatcccagccagcttccagccctctctagctccccagcgcacagtggcatgatgggaatcaactcctatggcagccagcttggggtcagcatctcagagtcaacacaaggaaataatcaagggtacatccgcaacacaagcagcatctctccgcggggatactcttccagctccacgcctcaacagtctaattacagtacctccagcaacagtatgaatggctacagcaatgtccccatggccaacttgggtgttccaggttcaccaggatttctaaatggctcacccaccggctctccttatggaatcatgtcatcaagtcccaccgttgggtcttccagcacatcctccatcctcccattttcctcttcagtttttcctgctgtcaaacagaagagtgcctttgcccctgtcatcaggccccaaggctccccttcacctgcctgctccagcggcaatggaaatggattcagagccatgaccggacttgttgtacccccgatgtaaagaagaactgctttcttatagcacaaaactacttactctgatggaccaataatgaagaaagcactaggagctcttttgggggtgtagtggtgcccccacatgaacatgatggacacccttgggtctgcaaggagccagcatcttacttggtcccacgtcctcctatagctctgatggtggctacacaaactgaccctcttgggacaaggacaaaagatgtcattgacgtagtcagtgctaagagcagaaatgcaattctttgttatgaacattatgaaaaccaccttcctatgtttgtaaaatatttaagaaaaaattggcaaacaattaatgcttaatattttggatactatttgtttttctttgtaggaaaaaaaagttgaaagtttctattttctatgaagcctttcagataccaatttagtttatgcagaaaaaaattgaacaaaacagggtaccagcacggaagactttcttaaaacgcaacctgaattgaatgatgaaatgttgtatgtgtgtttgcttatagcttaatctctttaaaaaatgaac

SEQ ID NO: 14 - protein, 575 aa, Homo sapiens
MFGIQDTLGRGPTLKEKSLGAEMDSVRSWVRNVGVVDANVAAQSGVALSRAHFEKQPPSNLRKSNFFHFVLALYDRQGQPVEIERTAFVDFVENDKEQGNEKTNNGTHYKLQLLYSNGVRTEQDLYVRLIDSVTKQPIAYEGQNKNPEMCRVLLTHEVMCSRCCEKKSCGNRNETPSDPVIIDRFFLKFFLKCNQNCLKTAGNPRDMRRFQVVLSTTVNVDGHVLAVSDNMFVHNNSKHGRRARRLDPSEATPCIKAISPSEGWTTGGAMVIIIGDNFFDGLQVVFGTMLVWSELITPHAIRVQTPPRHIPGVVEVTLSYKSKQFCKGAPGRFIYTALNEPTIDYGFQRLQKVIPRHPGDPERLAKEMLLKRAADLVEALYGTPHNNQDIILKRAADIAEALYSVPRNPSQLPALSSSPAHSGMMGINSYGSQLGVSISESTQGNNQGYIRNTSSISPRGYSSSSTPQQSNYSTSSNSMNGYSNVPMANLGVPGSPGFLNGSPTGSPYGIMSSSPTVGSSSTSSILPFSSSVFPAVKQKSAFAPVIRPQGSPSPACSSGNGNGFRAMTGLVVPPM

SEQ ID NO: 15 - DNA, 3727 bp, Homo sapiens
aagtgagagcagcggcagccggcggtgcagcagccggccgacccagagtgtaagtgcgtgtgctggggcgagcgggagcgggcgaggatgggcacaggatagaggcagagccacccacgccgccgcggccccacgctgggcgacagagcctccagttccccttcaatggtggcgggtcgccggagctctgatcgccgggaacccttgccgctgctgtcctgcgaccccaagcaggtatagacacgtgtggccgtttacgctgtaggatcctcattcccactggctttgaacattttggggacttacaatgccgccacccgcggacatcgtcaaggtggccatagaatggccgggcgcctaccccaaactcatggaaattgatcagaaaaaaccactgtctgcaataataaaggaagtctgtgatgggtggtctcttgccaaccatgaatattttgcactccagcatgccgatagttcaaacttctatatcacagaaaagaaccgcaatgagataaaaaatggcactatccttcgattaaccacatctccagctcagaacgcccagcagctccatgaacgaatccagtcctcgagtatggatgccaagctggaagccctgaaggacttggccagcctctcccgggatgtcacgtttgcccaggagtttataaacctggacggtatctctctcctcacgcagatggtggagagcggcactgagcgataccagaaattgcagaagatcatgaagccttgctttggagacatgctgtccttcaccctgacggccttcgttgagctgatggaccatggcatagtgtcctgggatacattttcggtggcgttcattaagaagatagcaagttttgtgaacaagtcagccatagacatctcgatcctgcagcggtccttggccattttggagtcgatggtgctcaatagccatgacctctaccagaaagtggcgcaggagatcaccatcggccagctcattccacacctgcaagggtcagatcaagaaatccaaacctatactattgcagtgattaatgcgcttttcctgaaggctcctgatgagaggaggcaggagatggcgaatattttggctcagaagcaactgcgttccatcattttaacacatgtcatccgagcccagcgggccatcaacaatgagatggcgcaccagctgtatgttctacaagtgctcacctttaacctcctggaagacaggatgatgaccaaaatggacccccaggaccaggctcagagggacatcatatttgaacttcgaagaattgcttttgatgctgagtctgaacctaacaacagcagtggcagcatggagaaacgcaagtccatgtacacgcgagattataagaagcttgggttcattaatcatgtcaaccctgccatggacttcacgcagactccacctgggatgttggctctggacaacatgctgtactttgccaagcaccaccaagatgcctacatccggattgtgcttgagaacagtagtcgagaagacaagcatgaatgtccctttggccgcagtagtatagagctgaccaagatgctatgtgagatcttgaaagtgggcgagttgcctagtgagacctgcaacgacttccacccgatgttcttcacccacgacagatcctttgaggagtttttctgcatctgtatccagctcctgaacaagacatggaaggaaatgagggcaacttctgaagacttcaacaaggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacggatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactgaagtggccgggcccagacatgccccttccaaaactggaacacctagctaacaggagagaggaatgaaaacacacccacgccttggaaccgtcctttggtaaagggaagctgtgggtccacattcccttcagcatcacctctagccctggcaactttcagcccctagctggcatcttgctcaccgccctgattctgttcctcggctccactgcttcaggtcacttcccatggctgcagtccactggtgggacaagagcaaagcccactgccagtaagaaggccaaagggcccttccatcctagccctctgcaggcatgcccttccttcccttgggcaggaaagccagcagccccagactgcccaaaaacttgcccaccagaccaagggcagtgccccaaggcccctgtctggaggaaatggcctagctatttgatgagaagaccaaaccccacatcctcctttcccctctctctagaatcatctcgcaccaccagttacacttgaattaagatctgcgctcaaatctcctcccacctctctccctgcttttgccttgctctgttcctctttggtcccaagagcagcagccgcagcctcctcgtgatcctccctagcataaatttcccaaacagtccacaggtcccatgcccactttgcgtctgcactgtgatcgtgacaaatcttccctcctcaccagctagtctggggtttcctctccctgccccaggccagaactgccttcttcatttccacccacgctcccagcctcttagctgaaagcacaaatggtgaaatcagtagtctcgctccatctctaatagactaaacctaaatgcctctaggacggactgttgctatccaagcgtttggtgttaccttctcctgggaggtcctgctgcaactcaagttccacaggatggtcaagctgtcagacatccaagtttacatcattgtaattattactggtatttacaatttgcaagagttttgggttagtttttttttttttttttgctttgtttttgtacaaaagagtctaacattttttgccaaacagatatatatttaatgaaaagaagagatacataaatgtgtgaatttccagtttttttttaattattttaatcccaaacatcttcctgaaaataacattcccttaaacatgctgtggaataaaatggattgtgatgatttggaaaaaaaaaaaaaaaaaaaaaaaaaaaaaaaaaaaaaaaaa

SEQ ID NO: 16 - protein, 727 aa, Homo sapiens
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 17 - DNA, 2129 bp, Homo sapiens
tggaggggcagcgtggggaacgagaaactctttgcctgtagggacccttctagctgcaaacttaaaaatgtatgtggcaagatgcaacccaagcaccgagcaggattccagacgagttataatcattctgaacggggagaggaaaggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacggatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactgaagtggccgggcccagacatgccccttccaaaactggaacacctagctaacaggagagaggaatgaaaacacacccacgccttggaaccgtcctttggtaaagggaagctgtgggtccacattcccttcagcatcacctctagccctggcaactttcagcccctagctggcatcttgctcaccgccctgattctgttcctcggctccactgcttcaggtcacttcccatggctgcagtccactggtgggacaagagcaaagcccactgccagtaagaaggccaaagggcccttccatcctagccctctgcaggcatgcccttccttcccttgggcaggaaagccagcagccccagactgcccaaaaacttgcccaccagaccaagggcagtgccccaaggcccctgtctggaggaaatggcctagctatttgatgagaagaccaaaccccacatcctcctttcccctctctctagaatcatctcgcaccaccagttacacttgaattaagatctgcgctcaaatctcctcccacctctctccctgcttttgccttgctctgttcctctttggtcccaagagcagcagccgcagcctcctcgtgatcctccctagcataaatttcccaaacagtccacaggtcccatgcccactttgcgtctgcactgtgatcgtgacaaatcttccctcctcaccagctagtctggggtttcctctccctgccccaggccagaactgccttcttcatttccacccacgctcccagcctcttagctgaaagcacaaatggtgaaatcagtagtctcgctccatctctaatagactaaacctaaatgcctctaggacggactgttgctatccaagcgtttggtgttaccttctcctgggaggtcctgctgcaactcaagttccacaggatggtcaagctgtcagacatccaagtttacatcattgtaattattactggtatttacaatttgcaagagttttgggttagtttttttttttttttttgctttgtttttgtacaaaagagtctaacattttttgccaaacagatatatatttaatgaaaagaagagatacataaatgtgtgaatttccagtttttttttaattattttaatcccaaacatcttcctgaaaataacattcccttaaacatgctgtggaataaaatggattgtgatgatttggaaaaaaaaaaaaaaaaaaaaaaaaaaaaaaaaaaaaaaaaa

SEQ ID NO: 18 - protein, 247 aa, Homo sapiens
MQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 19 - DNA, 2177 bp, Homo sapiens
catttaaaggtgtgcggcgggtctctgttcacatggctcaactggaaacctgtttcatgaacaagcttactcaggaaccatctggtggtattccagcacattgttcttcagggggacgactctaagtcgctttgtggtggcagcagcttagaatcagtatttgtggttgggaaagatggacttacgggagcttggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacggatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactgaagtggccgggcccagacatgccccttccaaaactggaacacctagctaacaggagagaggaatgaaaacacacccacgccttggaaccgtcctttggtaaagggaagctgtgggtccacattcccttcagcatcacctctagccctggcaactttcagcccctagctggcatcttgctcaccgccctgattctgttcctcggctccactgcttcaggtcacttcccatggctgcagtccactggtgggacaagagcaaagcccactgccagtaagaaggccaaagggcccttccatcctagccctctgcaggcatgcccttccttcccttgggcaggaaagccagcagccccagactgcccaaaaacttgcccaccagaccaagggcagtgccccaaggcccctgtctggaggaaatggcctagctatttgatgagaagaccaaaccccacatcctcctttcccctctctctagaatcatctcgcaccaccagttacacttgaattaagatctgcgctcaaatctcctcccacctctctccctgcttttgccttgctctgttcctctttggtcccaagagcagcagccgcagcctcctcgtgatcctccctagcataaatttcccaaacagtccacaggtcccatgcccactttgcgtctgcactgtgatcgtgacaaatcttccctcctcaccagctagtctggggtttcctctccctgccccaggccagaactgccttcttcatttccacccacgctcccagcctcttagctgaaagcacaaatggtgaaatcagtagtctcgctccatctctaatagactaaacctaaatgcctctaggacggactgttgctatccaagcgtttggtgttaccttctcctgggaggtcctgctgcaactcaagttccacaggatggtcaagctgtcagacatccaagtttacatcattgtaattattactggtatttacaatttgcaagagttttgggttagtttttttttttttttttgctttgtttttgtacaaaagagtctaacattttttgccaaacagatatatatttaatgaaaagaagagatacataaatgtgtgaatttccagtttttttttaattattttaatcccaaacatcttcctgaaaataacattcccttaaacatgctgtggaataaaatggattgtgatgatttggaaaaaaaaaaaaaaaaaaaaaaaaaaaaaaaaaaaaaaaaa

SEQ ID NO: 20 - protein, 247 aa, Homo sapiens; identical to SEQ ID NO: 18

SEQ ID NO: 21 - DNA, 3936 bp, Homo sapiens
aaattctgcaagtgaacttgactcaggaaggccagcggctcaaggtccagcccctggaagagagaatagctacagattctccatcctcagtctttgcaaggcgacagctgtgccagccgggctctggcaggctcctggcagcatggcagtgaagcttgggaccctcctgctggcccttgccctgggcctggcccagccagcctctgcccgccggaagctgctggtgtttctgctggatggttttcgctcagactacatcagtgatgaggcgctggagtcattgcctggtttcaaagagattgtgagcaggggagtaaaagtggattacttgactccagacttccctagtctctcgtatcccaattattataccctaatgactggccgccattgtgaagtccatcagatgatcgggaactacatgtgggaccccaccaccaacaagtcctttgacattggcgtcaacaaagacagcctaatgcctctctggtggaatggatcagaacctctgtgggtcactctgaccaaggccaaaaggaaggtctacatgtactactggccaggctgtgaggttgagattctgggtgtcagacccacctactgcctagaatataaaaatgtcccaacggatatcaattttgccaatgcagtcagcgatgctcttgactccttcaagagtggccgggccgacctggcagccatataccatgagcgcattgacgtggaaggccaccactacgggcctgcatctccgcagaggaaagatgccctcaaggctgtagacactgtcctgaagtacatgaccaagtggatccaggagcggggcctgcaggaccgcctgaacgtcattattttctcggatcacggaatgaccgacattttctggatggacaaagtgattgagctgaataagtacatcagcctgaatgacctgcagcaagtgaaggaccgcgggcctgttgtgagcctttggccggcccctgggaaacactctgagatatataacaaactgagcacagtggaacacatgactgtctacgagaaagaagccatcccaagcaggttctattacaagaaaggaaagtttgtctctcctttgactttagtggctgatgaaggctggttcataactgagaatcgagagatgcttccgttttggatgaacagcaccggcaggcgggaaggttggcagcgtggatggcacggctacgacaacgagctcatggacatgcggggcatcttcctggccttcggacctgatttcaaatccaacttcagagctgctcctatcaggtcggtggacgtctacaatgtcatgtgcaatgtggtgggcatcaccccgctgcccaacaacggatcctggtccagggtgatgtgcatgctgaagggccgcgccagcactgccccgcctgtctggcccagccactgtgccctggcactgattcttctcttcctgcttgcataactgatcatattgcttgtctcagaaaaaaacaccatcagcaaagtgggcctccaaagccagatgattttcattttatgtgtgaataatagcttcattaacacaatcaagaccatgcacattgtaaatacattattcttggataattctatacataaaagttcctacttgttaaaaaagatacaaaccttgtttttccagaaggtaggaaaatcctagctttccatttgtgcagttatatgtcattttctcctttcttttcacgttactcaggatgaactctctgagcagggacctgctcctgcagcaaccaaacttggagtggttattgcagacagacgtggctctgggcccctctctgtcccaccttgcacaaaggaccccctcagaccaggcccttgtctgtgccctgtccacacccaggagccatcctcagtgtctgtggccacaatcctgtactgttccttccatccctgataaaaggaggtctacatgaaagcaaaagctactgtctatttctgacccagctcatggaattttttcatcttatactgagctccagaaaggacgtaacttagcatggatcaccaatcaatcaaaaaataaataaatcactaaggattggagaactcatagaacaaggtgaaagacatgagtgccctcccaaagtctgagtgcacgaaaatttctctcttgccttgaggagcagaaaagcttctgatggacatgggcttctgtgagacttatcacacatagtgtatcgtggcatgaagcccggcacatagcaggccctgcatattgatggacaaatggatggcctgcctgccttccctgtccgttcacctgtgcaaaggcttcctcagacatgccactctgtggctcccaatatagggtgcagacaagagcaatccctgacatgacattatagcctgggaaagggctggctcactgatgagaatgtggaggcatcagcaaggatctcggtgggttgctcagagaggtgatgcactaagccttaatcctggacaccagtacccctgcagcatggcttgctcaacaacagtctttgagtggcatagaattccaaagaaaatggtgctgggtggagaatggagagagcatgatggagcagagtcccagtcactgaccaactaactggtcgtttgattaggaaacagtttggccaaagtaccacctttgagacctaagttcttttgatacctttgagaagagccactgagcctgagttgaaatatttttagcttagtcatctgtgtttgctataggagaaattgtaacacaagaaataactcctttttacatgatcatttatatctatatacatatatatacttgcatacactatcactgcattaaaaaatgagtttgggctgggcatggtggctcacacctataatcccaacactttcggaggccaaggagggacaaacgaacccttgaggccaggagttccagactaacttgggcaacacagggcgacccccatctctacaaaacataaaagatttttaaaaaattagccaggcatggtggcacatgcctgtggtctcagctacttgggaggctgaggcaggagaatcatttgagcccaggaggtcaaggctgcagtgagctttgatcacaccactgcactccagcctgggcaacagagcaagaccccatcctccaccccccccaaaaaatagaaagaaaaaaaaagtttgcactaattgaggtacatctgcaagtgagactttttgtcaggaaaaggcaatatatcaggtctcctcaggacgatggaggccttatatggtgtgttaccttgaaaactgaatatcaacgttcaccttgattcaggaaagctgggtgctgtctccatgccatgaatcatgagagcaaaggatcactgcttaaaaatactgaatttaccttcacaaaagatttctaaagatttatgtaatgtgttttaaaagcgccagtaaaccatcggatcaattggaaagaaggcaactcttcagcctttgttatctagctgaaaacaaatgacaactttcaaaacattggcagtagttgttgaaaaagacgtctattgttcaaagtttctttctccttaaaggacggtgttccaatgaattcagtagagcccactttcctccactgtggaggaagaatccctaagagatactcaaatgattaaattaaaattggatcatcaaactcaagagaggcataaacttagacacagtcttgcatttttgtctttcctgaactcttctgccattttcctccttcactcgtcctgaaaatctgcaagttacataataaaactttagatatttgtctgacaaagtgtaattactcaactgaataaatgactgagaacaagttacaaaaggaatcatgaatcctggtaaacaataaagaagattcagacactgagggaaaaaaataaagctttttacttaaataatgca

SEQ ID NO: 22 - protein, 440 aa, Homo sapiens
MAVKLGTLLLALALGLAQPASARRKLLVFLLDGFRSDYISDEALESLPGFKEIVSRGVKVDYLTPDFPSLSYPNYYTLMTGRHCEVHQMIGNYMWDPTTNKSFDIGVNKDSLMPLWWNGSEPLWVTLTKAKRKVYMYYWPGCEVEILGVRPTYCLEYKNVPTDINFANAVSDALDSFKSGRADLAAIYHERIDVEGHHYGPASPQRKDALKAVDTVLKYMTKWIQERGLQDRLNVIIFSDHGMTDIFWMDKVIELNKYISLNDLQQVKDRGPVVSLWPAPGKHSEIYNKLSTVEHMTVYEKEAIPSRFYYKKGKFVSPLTLVADEGWFITENREMLPFWMNSTGRREGWQRGWHGYDNELMDMRGIFLAFGPDFKSNFRAAPIRSVDVYNVMCNVVGITPLPNNGSWSRVMCMLKGRASTAPPVWPSHCALALILLFLLA

SEQ ID NO: 23 - DNA, 5084 bp, Homo sapiens
gggcgggggcgatgctgccggagccgccgccgccgccgccgcctcgatgagagccgcgccgcaccgctcatagccgcacaggctgacaggcaggaggaccgacttccctctcccgggcatcctccctgggctgccgggaggcggcggcggcggaggaggaggaggaacgaggggagaaggcggagagcaggaacgcgaggaggaggacctggatccgtttcctccggccaggacccgagcggccccagccaccgctacccgccggcgctgtccgctctccatcagccctcctgcgcccacccgcgaccccgggctctctgcgcgtcgggccggggccggagccgcgcgccggagactatctggcttcctggtgatgctcacgctttgctaagtgttggcggccatcgtggttttcgcatcctggggacgaatcctgagcttgccagagacgggcggcgcaaggtccgggctctgtttccctgtgagaagccgcctcggcccaccgagatgtcccggcaccatagccgcttcgaaagagattaccgggtgggctgggaccgccgcgaatggagcgtcaacgggacgcatgggaccaccagcatctgcagtgtcacctcgggggccggtggcggcacagccagcagcctcagcgtccggcccggcctcctgccgctgcccgtggtgccctcccggctgcccaccccggctacagctcctgctccctgcaccaccggcagcagcgaggccatcaccagcctcgtggccagctctgcgtctgcggtcaccaccaaggctcccggcatctccaaaggggacagtcagtcccagggactggcgaccagcatccggtgggggcagacgcctatcaatcagtccacaccctgggacactgatgagccaccctccaaacagatgagagagagtgacaatccaggcacagggccatgggtgaccacggtggccgccgggaaccagcccaccctgattgcacactcctatggagtggcccagcctcccaccttcagcccggctgtgaacgtccaggccccggtcattggggtgaccccctcactgcctccccacgtggggccccagctcccgctgatgccaggccactactcgctccctcagccgccctctcagccactgagcagcgtggtggtcaacatgcctgcccaggccctgtatgccagccctcagcccctggccgtgtccacactgcccggtgtggggcaggtggcccgcccaggacccaccgctgtgggcaacggccacatggcagggcccctgctgcctccaccgccgccagcccagccgtccgccactctccccagtggtgcccctgccaccaatgggccccccacaaccgactcggcccacgggctgcagatgctgcggaccattggcgtggggaagtatgagttcaccgacccggggcaccccagagaaatgttgaaggaattgaaccagcaacgcagagcgaaagcgtttacagacctgaaaattgttgttgaaggcagagagtttgaagtccaccaaaatgttctagcttcctgcagcttgtatttcaaggacctgattcaaaggtccgtgcaagacagcggccagggcggccgggagaagctggagctcgtcctgtcgaacctgcaggcagacgtcctggagttgctgctggagtttgtctacacgggctccctggtcatcgactcggccaacgccaagacactgctggaggcggccagcaagttccagttccacaccttctgcaaagtctgcgtgtcctttctcgagaagcagctgacggccagcaactgcctgggcgtgctggccatggccgaggccatgcagtgcagcgagctctaccacatggccaaggccttcgcgctgcagatcttccccgaggtggccgcccaggaggagatcctcagcatctccaaggacgacttcatcgcctacgtctccaacgacagcctcaacaccaaggctgaggagctggtgtacgagacagtcatcaagtggatcaagaaggaccccgcgacacgcacacagtacgcggctgagctcctggccgtggtccgcctccccttcatccaccccagctacctgctcaatgtggttgacaatgaagagctgatcaagtcatcagaagcctgccgggacctggtgaacgaggccaaacgctaccatatgctgccccacgcccgccaggagatgcagacgccccgaacccggccgcgcctctctgcaggtgtggctgaggtcatcgtcttggttgggggccgtcagatggtggggatgacccagcgctcgctggtggccgtcacctgctggaacccgcagaacaacaagtggtaccccttggcctcgctgcccttctatgaccgcgagttcttcagtgtagtgagtgcaggggacaacatctacctctcaggtgggatggaatcaggggtgacgctggctgatgtctggtgctacatgtccctgcttgataactggaacctcgtctccagaatgacagtcccccgctgtcggcacaatagcctcgtctacgatgggaagatttacaccctcgggggacttggcgtggcaggcaacgtggaccacgtggagaggtacgacaccatcaccaaccaatgggaggcggtggcccctctgcccaaggcagtacactctgctgcagccacagtgtgtggcggcaagatctacgtgtttggtggggtgaacgaggcaggccgagctgccggcgtcctccagtcttacgttcctcagaccaacacgtggagcttcatcgagtccccaatgattgacaacaagtatgcccccgctgtcacgctcaatggcttcgttttcatcctgggcggggcttatgccagagctaccaccatctacgaccctgagaaaggaaacattaaggcgggcccaaacatgaaccactctcgccagttctgcagtgctgtggtgcttgatggcaagatttatgcaactggaggtattgtcagcagtgaagggcccgcgctgggcaacatggaggcctacgagcccacaaccaacacatggaccctcctcccccacatgccctgccctgtgttcagacacggctgcgtcgtgataaagaaatatattcaaagcggctgacatcagcagaaagcccacgataagactgtggacaagtctggtgaggcaagtgccacgcaatgataattttccagcgacaccaacaagaggccaacaaaacacaatcaaggaactcactgcgctcaacatgttgaatattctctacattgaatgtagaaaatcatcctcgcctttggatgaaacggaggcaccgcgcttggagccgcaggaaccacgatcccgccatggggctggctgcctcctgaacaggggcgctcgctctgccaggtgcaatagagtttcacgtatttttcaactgggagagagaagctgttttttccttcctgcagagcaagcttgatccctaaacaaccatagatcagttatcttatgacaacattaggcatcaggctctcttggaataagatcaaagtgtccttatcactttgattcctacttttgttttttaaccgatctacactttcagtggccgacagaaaacgagggacaatactgtgcatcacaaggcctaggaggctgctggtccccactggggctgaagagaagcccagctgcccacgcggagccaggggtggcagctgtgggacagccggggagcagggacagcggtctgtccttcacaggtttttctactgtgtttttgctggagaaggacagtgattgcgctagctttctcttacccggtatgaattatttagatttctgaggcattttcttgataaacaaaaggctatttttaagtactgagaggaggagcaggccacaagagggataatgttgtgggaattcccaaagctctttgtaggtagtgccagaggggggcttttgctctcatttttctatgtgcagaatagaggatctctcctggggtgggcgatgcccccattttatttttagaaaaagtaactcccagacagccccataaaagctgtgcccaaggaagaagagtctgctctagaaggagcccggttctggctcaggacaccggcccagctccctccatgaggtcaagctgaggaccaggccagtgggaagggaaggagggagaattagcgtctataaagcacaggagactatttttgatattcatagctatatattaaggcacctgccacaagagctctcaggatggggacagccttcttagtggagccatggcagcaaggcctgagggcatgaacagaaccactcttcttgtcacatacgaacctgagaaaagggaagccaggagggaggtcacaccatggctcaaaagggaaaggccttcccacttgtccttagcccctcaaacctcacacggtcaacagtttccattccagggcaggagaatgctgccgccactgcgctgttgagttgaagttggtaccaaatacacatttaccacttttatatctgggaagtcaacttgccatcgtttcatgataacaaccatttataagagaaaaagacaggacacgctttccatcgttcagtatttgatgacacaaaattccagttctaacgttgggcatcaacttctagcactacgagtgtggctcccacttggacaagataccgagcttcgttatgcagtttttaatattatttattattttaaaaagtaataagcacaaaactacatacattgtatgtcatttaaagtatttatgtcaaacagggtgcaagtgtgaacccaaggactggagcacaaattcctaactgcctggggcagggctaatgttagcattggtgtgcgtctgcctccaaaggaggttctagttgtcagcgagactcaacacagatgacattgaaattcgtttctctcctcatctatcacactggagcaaaactggctatttctgtgaatgatataaaacagggttctctgtaatggtattgtacatagtatatgtttactgttaagttcttgttatattataataaatatatttatagatctagacttggaa

SEQ ID NO: 24 - protein, 875 aa, Homo sapiens
MSRHHSRFERDYRVGWDRREWSVNGTHGTTSICSVTSGAGGGTASSLSVRPGLLPLPVVPSRLPTPATAPAPCTTGSSEAITSLVASSASAVTTKAPGISKGDSQSQGLATSIRWGQTPINQSTPWDTDEPPSKQMRESDNPGTGPWVTTVAAGNQPTLIAHSYGVAQPPTFSPAVNVQAPVIGVTPSLPPHVGPQLPLMPGHYSLPQPPSQPLSSVVVNMPAQALYASPQPLAVSTLPGVGQVARPGPTAVGNGHMAGPLLPPPPPAQPSATLPSGAPATNGPPTTDSAHGLQMLRTIGVGKYEFTDPGHPREMLKELNQQRRAKAFTDLKIVVEGREFEVHQNVLASCSLYFKDLIQRSVQDSGQGGREKLELVLSNLQADVLELLLEFVYTGSLVIDSANAKTLLEAASKFQFHTFCKVCVSFLEKQLTASNCLGVLAMAEAMQCSELYHMAKAFALQIFPEVAAQEEILSISKDDFIAYVSNDSLNTKAEELVYETVIKWIKKDPATRTQYAAELLAVVRLPFIHPSYLLNVVDNEELIKSSEACRDLVNEAKRYHMLPHARQEMQTPRTRPRLSAGVAEVIVLVGGRQMVGMTQRSLVAVTCWNPQNNKWYPLASLPFYDREFFSVVSAGDNIYLSGGMESGVTLADVWCYMSLLDNWNLVSRMTVPRCRHNSLVYDGKIYTLGGLGVAGNVDHVERYDTITNQWEAVAPLPKAVHSAAATVCGGKIYVFGGVNEAGRAAGVLQSYVPQTNTWSFIESPMIDNKYAPAVTLNGFVFILGGAYARATTIYDPEKGNIKAGPNMNHSRQFCSAVVLDGKIYATGGIVSSEGPALGNMEAYEPTTNTWTLLPHMPCPVFRHGCVVIKKYIQSG

SEQ ID NO: 25 - DNA, 4434 bp, Homo sapiens
atatttggactcggctgcccgtgcccaggaatttcccgtcatgcctcccgccgccccgtccgtcgcccggagccggggagggagggagcgaggttcggacaccggcggcggctgcctggcctttccatgagcccgcggcggaccctcccgcgccccctctcgctctgcctctccctctgcctctgcctctgcctggccgcggctctgggaagtgcgcagtccgctcttgcccctgttctttgcttctcgttttgttggtgaagatatcacagtgatgtctgcattcaacctgctgcatttggtgacaaagagccagccagtagcccttcgagcctgtgggcttccctcagggtcgtgtagggataaaaagaactgtaaggtggtcttttcccagcaggaactgaggaagcggctaacacccctgcagtaccatgtcactcaggagaaagggaccgaaagtgcctttgaaggagaatacacacatcacaaagatcctggaatatataaatgtgttgtttgtggaactccattgtttaagtcagaaaccaaatttgactccggttcaggttggccttcattccacgatgtgatcaattctgaggcaatcacattcacagatgacttttcctatgggatgcacagggtggaaacaagctgctctcagtgtggtgctcaccttgggcacatttttgatgatgggcctcgtccaactgggaaaagatactgcataaattcggctgccttgtcttttacacctgcggatagcagtggcaccgccgagggaggcagtggggtcgccagcccggcccaggcagacaaagcggagctctagagtaatggagagtgatggaaacaaagtgtacttaatgcacagcttattaaaaaaatcaaaattgttatcttaatagatatattttttcaaaaactataagggcagttttgtgctattgatattttttcttcttttgcttaaacagaagccctggccatccatgtattttgcaattgactagatcaagaactgtttatagctttagcaaatggagacagctttgtgaaacttcttcacaagccacttataccctttggcattcttttctttgagcacatggcttcttttgcagtttttccccctttgattcagaagcagagggttcatggtcttcaaacatgaaaatagagatctcctctgcagtgtagagaccagagctgggcagtgcagggcatggagacctgcaagacacatggccttgaggcctttgcacagacccacctaagataaggttggagtgatgttttaatgagactgttcagctttgtggaaagtttgagctaaggtcatttttttttttctcactgaaagggtgtgaaggtctaaagtctttccttatgttaaattgttgccagatccaaaggggcatactgagtgttgtggcagagaagtaaacattaccacactgttaggcctttattttattttattttccatcgaaagcattggaggcccagtgcaatggctcacgcctgtgatcccagcactttgggaggccaaggcgggtggatcacgaggtcaggagatggagaccatcctggctaacatggtgaaaccccgtctctactaaaaatacgaaaaattagccaggcgtggtggtgggcacctgtagtcccagctactcaggaggctgaggcaggagaatggcgtgaacccggaaggcggagcttgcagttagccgagatcatgccactgcactccagcctacatgacaatgtgacactccatctcaaaaaataataataataacaatataagaactagctgggcatggtggcgcatgcatgtagtcccagctactcctgaggctcagtcaggagaatcgcttgaacttgggaggcggaggttgcagtgagctgagctcataccactgcactccagcctgaacagagtgagatcctgtcaaaaaagaaaagaaaaagaaagcagcattcaaatgtaagacaactgtaaaatattgagccccacttggtctaaaattcaaaaagaagaacgcctgtccatcgcctttttataagtccttctctccacacctaaaagcagctgcagctggaagggcacaaattccactgtgtaaaataaaatattaggggcaacacacttcatcaaggcagcaggaatgagagagagcagagaagatcaaggatgaagtcttgggtactgaaaaattcagtgctgggcagaaaaactgacagggcagtacaagtaacaaacagaatccaagtggggtggcccttgtgcacagagctccaggtgacctctggagagacatgggcattcacatggaaagctaaaacggaagctcaagtttcatactcaacataatcttctgtgtgacaaaggacaagccatgtagcctctctgtgcctatttcttcatgcataaactgggactcataatatttgtaaaatgtattgatactctcagggcaaattcactatattgctatacagttgagatcagtgttgtaaaattaaactgatctggttctaattgcctcaaaggccaaagcccaggcatttgaaatggaaagaagcagagaggaggctgacttagctgattggtatggaaacagttgggccaagagccagaatttccctttgtagcaacacggctagttttactttgagaagctctgctcagctgctttataacattaagtctggcggaatggatgtcactgtgcacaataaagttttcacaagtataaacaatggtgatgtaagtcaacattgctgtagccaggtgtgaaggttgtatggtgtgtgacgaatgtacatcatgtttgtaggtttggatgctaatcttgaattgtagtttaaaaaatacgtatttttgtaactctttgaaagtttatgaagactgacagctttccttgtaagcactaagagaaaaaaaagaaagagggacatttgacaattttaaagaaacaacaagaaattagaatgaaaatctgtgacaaacagcgtcagtgtggccatgtccacattcctacatgtctctctctacaagcacctctctaagaagcctgacatcccggtggactctttatagtcatgtacacttgattccagatgagctctggtcttatctggatgctcagataagaggtttctatctgagcatccagatgttccctcaggttccaagacatttcaccccaggccctgggttcactctggaattcgtaggcttcacgtctctctagaaatgacgtgtaaaatttaagaccagacctcagccatcagcgtccagaccatcctagaagtctttcccaatctcacagagaaagccctagtatttcccagtgaccccaggattccacgttggggtggccaaagaaataggtctctcagggctttgccacagcctccagcccatccttcagaggcacacacagcacctctcggctgctccagctctgtaggatagcctcccctggggtccgtgggacgcgggccacagtgttgaggtagacaaggaggatcagtgagaggcctcttccctctccacagagactggattgtcattgttccttcatttatatcgtagggcttaacatttcactcaaaaaaaagcccctctttttctaatccttagtctttgtttcaaggaaagccagtttttcttctaccacattttccaggatcgactttaagaaaaatgcaacatctattgaaaaaaagtggggtgtatgcatgtggtttaattccagattgcttttgggtttaagtggtatcaaatttcagtatatttctgtcttatgtgaaagaaatatattactaaaacgtcagtgagcaataatgtcagctgtcaagcactagatttatttttgcaggatatggagtgcaatgaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttgaaaaatgacatgaattttcattaaaaacccttttcctatgtttattgtatacaagaattatgcaataaaatttctttataaaaataaaaaaaaaaaaaaaaaa

SEQ ID NO: 26 - protein, 185 aa, Homo sapiens
MSAFNLLHLVTKSQPVALRACGLPSGSCRDKKNCKVVFSQQELRKRLTPLQYHVTQEKGTESAFEGEYTHHKDPGIYKCVVCGTPLFKSETKFDSGSGWPSFHDVINSEAITFTDDFSYGMHRVETSCSQCGAHLGHIFDDGPRPTGKRYCINSAALSFTPADSSGTAEGGSGVASPAQADKAEL

SEQ ID NO: 27 - DNA, 9520 bp, Homo sapiens
gcctcctccccacacctgggaggggagtggtgcggcgcggcctcctcccccggcgctcgcaactcctgtccggccgtagctgcgccgccgcggcgggagtaaaggtcgcgccgccgggagcgagccggccgcggcgcctgcgggaagccggcggggcaggtcggagaagagcgagaagatcgagaaactccaggccagcccgggaacatggcgccaggcgggccagccgcggactgagagccgcggggcagccaggagccggggcccgagccccgcccggcccgggccatgtcggtgggcgagctctacagccagtgcacaagggtctggatccctgaccctgatgaggtatggcgctcagctgagttaaccaaggactacaaagaaggagacaagagcctacagctcagactggaggatgaaacgattctggaatacccaattgatgtacaacgcaaccagctgcccttcttacggaatccagatatcttggtgggagaaaatgacctgactgcccttagctatcttcatgagcctgcagttttgcataatttgaaggtccgtttcctggagtccaaccatatctacacttactgtggtatcgtacttgttgccattaatccttatgaacagttgccaatctatggacaagatgtcatctatacctacagtggccaaaacatgggagacatggacccccacatctttgctgtggcagaagaagcctacaagcagatggccagagatgagaagaatcagtccatcatagtcagtggggagtctggagccgggaagacggtatcagccaagtatgccatgcgctatttcgccaccgttggtggctcggccagtgaaaccaacatcgaagagaaggtgctggcatccagtcccatcatggaggccattggaaatgccaagaccacccgcaatgacaacagcagccgttttggcaagtacatccagattggctttgacaaaaggtaccacatcatcggggccaacatgaggacttacctcttggagaagtccagagtggtcttccaggcagatgatgagaggaattaccacatcttttaccagctctgtgctgctgccggtcttccagaatttaaagagcttgcactaacaagtgcagaggactttttctatacatcacagggaggagacacttccatcgagggtgtggacgatgctgaggactttgagaagactcgacaagccttcacactcctcggagtgaaagagtcccatcagatgagcatttttaagataattgcttctatcttgcaccttggaagtgtggcgattcaggctgagcgtgatggtgattcctgtagtatatcaccccaggatgtatacctaagcaacttctgccgactgctaggggtggagcacagtcagatggagcactggctgtgtcatcgcaagctggtcaccacctcggagacctacgtcaagaccatgtccctgcagcaggtgatcaatgcgcgcaacgccctggcgaagcacatctatgcccagttgttcggctggattgtggagcacatcaacaaggccctgcacacctccctcaagcagcactccttcatcggggtcctggacatctatgggtttgagacatttgaggtaaacagctttgagcagttctgtatcaactatgcaaatgaaaagctccagcagcagttcaactcgcatgttttcaaactggagcaagaagaatacatgaaggaacagatcccttggaccctgattgatttttatgataaccaaccttgtatcgacctcattgaagccaagctgggtatcttggacctgttggatgaagaatgtaaggtccccaaaggaactgaccagaactgggctcagaagctctatgaccggcactccagcagccagcacttccagaagccccgcatgtccaacacggccttcatcatcgtccactttgcagacaaggtggagtacctctctgatggttttctggagaaaaacagagacacggtgtatgaagagcagatcaatatcctgaaggccagcaagttcccactagtggctgacttgtttcatgatgacaaggaccctgttcctgccaccacccctgggaaggggtcatcttcgaagatcagcgtccgttctgccagaccccccatgaaagtctccaacaaggagcacaagaaaaccgttggccaccagttccgtacctccctgcatctgctcatggagaccctgaatgccacgacacctcactatgtccgctgcatcaagcccaacgatgagaagctcccctttcactttgacccaaagagagcagtgcagcaactcagagcctgcggggtgttggagacgattcgaatcagtgcagctggctacccatccaggtgggcctaccatgactttttcaaccggtatcgggtgctggtcaagaagagagagctcgccaacacagacaaaaaggccatctgcaggtctgtcctggagaacctcatcaaggaccccgacaagttccagtttggccgcaccaagatcttctttcgagcaggccaggtggcctacctggagaagctgcgggctgacaagttccggacagccaccatcatgatccagaaaactgtccggggatggctgcagaaggtgaaatatcacaggctgaagggggctaccttaaccctgcagaggtactgccggggacacctggcccgcaggctggctgagcacctgcggaggatcagagcggctgtggtgctccagaaacattaccgcatgcagagggcccgccaggcctaccagagggtccgcagagctgccgttgttatccaggccttcacccgggccatgtttgtgcggagaacctaccgccaggtcctcatggagcacaaggccaccaccatccagaagcacgtgcggggctggatggcacgcaggcacttccagcggctgcgggatgcagccattgtcatccagtgtgccttccggatgctcaaggccaggcgggagctgaaggccctcaggattgaggcccgctcagcagagcatctgaaacgtctcaacgtgggcatggagaacaaggtggtccagctgcagcggaagatcgatgagcagaacaaagagttcaagacactttcagagcagttgtccgtgaccacctcaacatacaccatggaggtagagcggctgaagaaggagctggtgcactaccagcagagcccaggtgaggacaccagcctcaggctgcaggaggaggtggagagcctgcgcacagagctgcagagggcccactcggagcgcaagatcttggaggacgcccacagcagggagaaagatgagctgaggaagcgagttgcagacctggagcaagaaaatgctctcttgaaagatgagaaagaacagctcaacaaccaaatcctgtgccagtctaaagatgaatttgcccagaactctgtgaaggaaaatctcatgaagaaagaactggaggaggagcgatcccggtaccagaaccttgtgaaggaatattcacagttggagcagagatacgacaaccttcgggatgaaatgaccatcataaagcaaactccaggtcataggcggaacccatcaaaccaaagtagcttagaatctgactccaattacccctccatctccacatctgagatcggagacactgaggatgccctccagcaggtggaggaaattggcctggagaaggcagccatggacatgacggtcttcctgaagctgcagaagagagtacgggagctggagcaggagaggaaaaagctgcaagtgcagctggagaagagagaacagcaggacagcaagaaagtccaggcggaaccaccacagactgacatagatttggacccgaatgcagatctggcctacaatagtctgaagaggcaagagctggagtcagagaacaaaaagctgaagaatgacctgaatgagctgaggaaagccgtggccgaccaagccacgcagaataactccagccacggctccccagatagctacagcctcctgctgaaccagctcaagctggcccacgaggagctcgaggtgcgcaaggaggaggtgctcatcctcaggacccagatcgtgagcgccgaccagcggcgactcgccggcaggaacgcggagccgaacattaatgccagatcaagttggcctaacagtgaaaagcatgttgaccaggaggatgccattgaggcctatcacggggtctgccagacaaacagcaagactgaggattggggatatttaaatgaagatggagaactcggcttggcctaccaaggcctaaagcaagttgccaggctgctggaggctcagctgcaggcccagagcctggagcatgaggaggaggtggagcatctcaaggctcagctcgaggccctgaaggaggagatggacaaacagcagcagaccttctgccagacgctactgctctccccagaggcccaggtggaattcggcgttcagcaggaaatatcccggctgaccaacgagaatctggaccttaaagaactggtagaaaagctggaaaagaatgagaggaagctcaaaaagcaactgaagatttacatgaagaaagcccaggacctagaagctgcccaggcattggcccagagtgagaggaagcgccatgagctcaacaggcaggtcacggtccagcggaaagagaaggatttccagggcatgctggagtaccacaaagaggacgaggccctcctcatccggaacctggtgacagacttgaagccccagatgctgtcgggcacagtgccctgtctccccgcctacatcctctacatgtgcatccggcacgcggactacaccaacgacgatctcaaggtgcactccctgctgacctccaccatcaacggcattaagaaagtcctgaaaaagcacaatgatgactttgagatgacgtcattctggttatccaacacctgccgccttcttcactgtctgaagcagtacagcggggatgagggcttcatgactcagaacactgcaaagcagaatgaacactgtcttaagaattttgacctcaccgaataccgtcaggtgctgagtgacctttccattcagatctaccagcagctcattaaaattgccgagggcgtgttacagccgatgatagtttctgccatgttggaaaatgagagcattcagggtctatctggtgtgaagcccaccggctaccggaagcgctcctccagcatggcagatggggataactcatactgcctggaagctatcatccgccagatgaatgcctttcatacagtcatgtgtgaccagggcttggaccctgagatcatcctgcaggtattcaaacagctcttctacatgatcaacgcagtgactcttaacaacctgctcttgcggaaggacgtctgctcttggagcacaggcatgcaactcaggtacaatataagtcagcttgaggagtggcttcggggaagaaaccttcaccagagtggagcagttcagaccatggaacctctgatccaagcagcccagctcctgcaattaaagaagaaaacccaggaggacgcagaggctatctgctccctgtgtacctccctcagcacccagcagattgtcaaaattttaaacctttatactcccctgaatgaatttgaagaacgggtaacagtggcctttatacgaacaatccaggcacaactacaagagcggaatgaccctcagcaactgctattagatgccaagcacatgtttcctgttttgtttccatttaatccatcttctctaaccatggactcaatccacatcccagcgtgtctcaatctggaattcctcaatgaagtctgaagatgcatgtttccagcattagtttgattcccaatgtgagcaagaaggaagtatatacagtaaagtaaattcaaggatctgttaaatctggtaaaagtagatcaaatcagagattgacagcctgtggagggtgctgaactatacagaattagacacaactatgtcattattttttgtacctactgctcagaataaaaacacttgaaatatggaagattttaagtttgatttcagtccaacacatatacataatttatagacaccaagcagtccccatagacatataaaaggtgtcaattctataaaacgaagctgcctagttttgatctttgcatagaactagagaatgtccaaattaaaataccaaatatatataagtcacataaattgccttcaaagggctttaacaaataatggtactaataaccatgataatggcatatactgacatttcccaaagtttgcaaaccataggtgtggttgagtttgtggtgagatgttttaagaacaaaaatatggggatgagacttctgagaaatattcccaaaatattttttaatggctgattatacacagacagtggtgtaactgacctccagaccagacattttgagtactggtttctgaagcaaaattagaagtgccagtcctcagtgtgctcaaacgcttttgtgttatcttgatttaatggaagagattattaaaatgctgctatcccaaactccaagtgagaaagatggaaaaatattttgtttctgatgctagtccatacactttccaagtcccacaaaactttcacaaaaatgtatataagctaaatattagaaacggataacaaacttgttttatttatagatgtaaaaaccaaacaagtcaatatgaaagcttttaatctcttaataccattaagcttccagtaagagcatcacataatgctctactgttccagaaaccaaatagtaaaacaaactaaagttcgcacatcagatcatctgaaaaaccttcaaaaataatcagttcagggatattatacaaaagtttgggttttttttttttaagagaataaaatggcttaggtcaactttcccttttcaggttattttcaacgtttttcaaatttagcacacaaaaaattgtaaatatctctcccacaaaataaggattttaaaaaagtaattcagtaatataacaggcttagatgtttgctgctcttagaattttttttaacttgtttttggtttcttcaaaagcaagcattcaattggaaacccatattctttccacacttttttttactgtcttttctgtatttcttgatagcagtatgctgttcccataagaaaaaaatggtatttgcaaatcatggaagaacagcctctgtattacattgagaaaataagatttatccatgaattggaagtagaacagcctgccttcaccctcttttactcaaccacccaacttaaaaggctcttggaaacacagcacactccacgctaccttctgcactgtgcccttagagcacagcttcctcagttgttctctgcatctcctggggcttaggccagtcttagcctgggttaaggctgctgacattgtgttccaatcagttgtcatgggcattatcccctctacatccacattaaacatgccggcttctcttgggcatcggcagagctgtgcctttttctttcagttacagttacataatcactgacgtccatgacacttacccatggatccatgtgctgacttcatttagaaggccaatctaaaacaactgggtttgtggctacctctttaaagttgtttgtgaaggataatttgttttttaatgcactttagtttgaaagtgagtctcttatgtaaggaccatccttaaaagaccaaaaatgccttgttagagtgttaaggagttttgacatgcagtggttccacaaacacagtggcttactatccttatacactgtcttataccatcattctctccatctctcttggtcactactctctgctgtcactggttaatcactaggtgccaagagcttactgaataaaagcttggcaattagaataaatggggagggaaggaccttatgaatagtccatttagcctaagaaatggcagatttagttcttctcttccaaaagataaaggtatatcctggaattgtacttaaaacttacagatgactaacaaatatatactttatatgtagttaatatttagatctgtcttatttaatacttggaggctagaagaagcatctttaggggaactatataatcttttgttagcattttctctgcattttaaaaaatcatttcaattcaaacatttatcagtgtcatgaaatcagtaatgactctttaacaattcaggtttgaactctgcattagatgtctctttaattttttaatatttaaaatttagttgacatttttttcaccaggtgcctttagcggttactaagataactgacatcagttgtttctctgaaataagtgttgctgtgggaataattttaatgttcaaggtgatatcatgggggagttttgtcttttaaaacattagaagcattttaaatattaagaatcaaatatttatagatcaaaacttgtgttttaagtattatacgggacctgtttacttatagtaaatgtgaatgtacacatgagttgttgctgaagctgacaagcatattacatacatgcattttccctgtgccctcatagttgcagttagagttccagtacctgtaggctcacctgggaggcagattagacccaaaggtagatgtttttcccctttccatgaagcatgtcagtgggagttgcttcctttgatttccctagtactaaattttaaggcttttgtaaaaacaaaacaaaactaggagcttggaacagttaaaaatcaacactgctaccatcaattcatcaaatatttacttagagctttcatacattaagattccagtaaccaataaattagaattcatttcttctgcataaagtaaattttcatacacttgacctactaagacagcaagggtgtcctaaattgaggcatttgtataatgcctgcataactaaatggtcactaaaatgggacagcatggggcaagaccttgtagttcttcacagaatatttgtggtcagtttctccaattaatttgctgcatgagccaaataaccataattcactttttatacccactggtgccataattagagaattagagggtgtagacagaggttaatgccaatgagaaacacaggacagggttttttttattataaaggtcattagatacaaaagattgtttttcaaaaaatttctaattctaacaaaggggatcaatcagaaatgaaactaagctactttctaaagtgacactgtatcagaataatccagatttgaatataacattttgccaccaactgacatttagatgaaggactgcctctctgaaagagttcagatcatattcaggggtgaatccaacaccatggaagaaagactactgatgaaaatattttcccactttgcacaaatctgtaaactacacctttgtttatagaaaaatgcttgtaatagtcactgtaatatttagctgtggataaaaatttgtggaaataaatacttttgaataaagaggtgtgccaaatctaaatgaaattt

SEQ ID NO: 28 - protein, 1848 aa, Homo sapiens
MSVGELYSQCTRVWIPDPDEVWRSAELTKDYKEGDKSLQLRLEDETILEYPIDVQRNQLPFLRNPDILVGENDLTALSYLHEPAVLHNLKVRFLESNHIYTYCGIVLVAINPYEQLPIYGQDVIYTYSGQNMGDMDPHIFAVAEEAYKQMARDEKNQSIIVSGESGAGKTVSAKYAMRYFATVGGSASETNIEEKVLASSPIMEAIGNAKTTRNDNSSRFGKYIQIGFDKRYHIIGANMRTYLLEKSRVVFQADDERNYHIFYQLCAAAGLPEFKELALTSAEDFFYTSQGGDTSIEGVDDAEDFEKTRQAFTLLGVKESHQMSIFKIIASILHLGSVAIQAERDGDSCSISPQDVYLSNFCRLLGVEHSQMEHWLCHRKLVTTSETYVKTMSLQQVINARNALAKHIYAQLFGWIVEHINKALHTSLKQHSFIGVLDIYGFETFEVNSFEQFCINYANEKLQQQFNSHVFKLEQEEYMKEQIPWTLIDFYDNQPCIDLIEAKLGILDLLDEECKVPKGTDQNWAQKLYDRHSSSQHFQKPRMSNTAFIIVHFADKVEYLSDGFLEKNRDTVYEEQINILKASKFPLVADLFHDDKDPVPATTPGKGSSSKISVRSARPPMKVSNKEHKKTVGHQFRTSLHLLMETLNATTPHYVRCIKPNDEKLPFHFDPKRAVQQLRACGVLETIRISAAGYPSRWAYHDFFNRYRVLVKKRELANTDKKAICRSVLENLIKDPDKFQFGRTKIFFRAGQVAYLEKLRADKFRTATIMIQKTVRGWLQKVKYHRLKGATLTLQRYCRGHLARRLAEHLRRIRAAVVLQKHYRMQRARQAYQRVRRAAVVIQAFTRAMFVRRTYRQVLMEHKATTIQKHVRGWMARRHFQRLRDAAIVIQCAFRMLKARRELKALRIEARSAEHLKRLNVGMENKVVQLQRKIDEQNKEFKTLSEQLSVTTSTYTMEVERLKKELVHYQQSPGEDTSLRLQEEVESLRTELQRAHSERKILEDAHSREKDELRKRVADLEQENALLKDEKEQLNNQILCQSKDEFAQNSVKENLMKKELEEERSRYQNLVKEYSQLEQRYDNLRDEMTIIKQTPGHRRNPSNQSSLESDSNYPSISTSEIGDTEDALQQVEEIGLEKAAMDMTVFLKLQKRVRELEQERKKLQVQLEKREQQDSKKVQAEPPQTDIDLDPNADLAYNSLKRQELESENKKLKNDLNELRKAVADQATQNNSSHGSPDSYSLLLNQLKLAHEELEVRKEEVLILRTQIVSADQRRLAGRNAEPNINARSSWPNSEKHVDQEDAIEAYHGVCQTNSKTEDWGYLNEDGELGLAYQGLKQVARLLEAQLQAQSLEHEEEVEHLKAQLEALKEEMDKQQQTFCQTLLLSPEAQVEFGVQQEISRLTNENLDLKELVEKLEKNERKLKKQLKIYMKKAQDLEAAQALAQSERKRHELNRQVTVQRKEKDFQGMLEYHKEDEALLIRNLVTDLKPQMLSGTVPCLPAYILYMCIRHADYTNDDLKVHSLLTSTINGIKKVLKKHNDDFEMTSFWLSNTCRLLHCLKQYSGDEGFMTQNTAKQNEHCLKNFDLTEYRQVLSDLSIQIYQQLIKIAEGVLQPMIVSAMLENESIQGLSGVKPTGYRKRSSSMADGDNSYCLEAIIRQMNAFHTVMCDQGLDPEIILQVFKQLFYMINAVTLNNLLLRKDVCSWSTGMQLRYNISQLEEWLRGRNLHQSGAVQTMEPLIQAAQLLQLKKKTQEDAEAICSLCTSLSTQQIVKILNLYTPLNEFEERVTVAFIRTIQAQLQERNDPQQLLLDAKHMFPVLFPFNPSSLTMDSIHIPACLNLEFLNEV

SEQ ID NO: 29 - DNA, 5675 bp, Homo sapiens
ccggcggcgtcccggggccaggggggtgcgcctttctccgcgtcggggcggcccggagcgcggtggcgcggcgcgggaggggttttctggtgcgtcctggtccaccatggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtgaggccggggccctccctcctgcactggccttggacggtattgcctgtcagtgaaataaataaagtcctgaccccagtgcacagacatagaggcacagattgcaaaaaaaaaaaaaaaaaaaaaaaaaaaaaaaaaaaaaaaaaaaa

SEQ ID NO: 30 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 31 - DNA, 5474 bp, Homo sapiens
ccggcggcgtcccggggccaggggggtgcgcctttctccgcgtcggggcggcccggagcgcggtggcgcggcgcgggaggggttttctggtgcgtcctggtccaccatggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtgaggccggggccctccctcctgcactggccttggacggtattgcctgtcagtgaaataaataaagtcctgaccccagtgcacagacatagaggcacagattgcaaaaaaaaaaaaaaaaaaaaaaaaaaaaaaaaaaaaaaaaaaaa

SEQ ID NO: 32 - protein, 1740 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 33 - DNA, 5577 bp, Homo sapiens
ccggcggcgtcccggggccaggggggtgcgcctttctccgcgtcggggcggcccggagcgcggtggcgcggcgcgggaggggttttctggtgcgtcctggtccaccatggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtgaggccggggccctccctcctgcactggccttggacggtattgcctgtcagtgaaataaataaagtcctgaccccagtgcacagacatagaggcacagattgcaaaaaaaaaaaaaaa

SEQ ID NO: 34 - protein, 1784 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV